(19) United States
(12) Patent Application Publication
(10) Pub. No.: US 2026/0176282 A1
(43) Pub. Date: Jun. 25, 2026

(54) BICYCLIC COMPOUNDS AS CDK INHIBITORS

(71) Applicant: BEONE MEDICINES I GMBH, Basel (CH)

(72) Inventors: Changxin Huo, Beijing (CN); Yu Zhao, Beijing (CN); Honglei Li, Beijing (CN); Huaqing Liu, Beijing (CN); Zhiwei Wang, Beijing (CN)

(73) Assignee: BEONE MEDICINES I GMBH, Basel (CH)

(21) Appl. No.: 19/539,172

(22) Filed: Feb. 13, 2026

Related U.S. Application Data
(63) Continuation of application No. PCT/IB2024/058177, filed on Aug. 22, 2024.

(30) Foreign Application Priority Data
Aug. 24, 2023 (WO) PCT/CN2023/114739

(51) Int. Cl. C07D 487/04

(52) U.S. Cl. CPC C07D 487/04

(57) ABSTRACT

Disclosed herein are bicyclic compound derivatives used as inhibitors of cyclin-dependent kinases. Disclosed herein is the use of these inhibitors for inhibiting cyclin-dependent kinases, and the use of such compounds for treating cancer.

### CROSS REFERENCE TO RELATED APPLICATIONS

This application is a continuation of International Application No. PCT/IB2024/058177, filed on Aug. 22, 2024, which claims priority to International Application No. PCT/CN2023/114739, filed on Aug. 24, 2023, each of which is incorporated herein by reference in its entirety.

### FIELD OF THE INVENTION

Disclosed herein are bicyclic compound derivatives used as inhibitors of cyclin-dependent kinases. Disclosed herein is the use of these inhibitors for inhibiting cyclin-dependent kinases, and the use of such compounds for treating cancer.

### BACKGROUND OF THE INVENTION

Cyclin-dependent kinases (CDKs), a family of Ser/Thr protein kinases, function as a driver of the cell cycle (Norbury, C, and Nurse, P., Annu Rev Biochem. 61, 441-470). During the cell cycle, CDK4 and CDK6 initiate the retinoblastoma (Rb) phosphorylation by binding to cyclinD and partially release the transcription factor E2F. Then the CDK2 forms a complex with cyclinE to fully phosphorylate Rb, entirely release E2F and initiate S-phase (Harbour, J. W. et al., Cell, 98(6), 859-869). Then in the S phase, CDK2 forms a complex with cyclinA (Echalier, A. et al., Biochim Biophys Acta, 1804(3), 511-519). Interestingly, in the normal cells, CDK2 is mostly not essential for the cell cycle, while in some cancer cells. CDK2 kinase activity plays a critical role in the abnormal growth process (Caruso, J. A. et al., Cancer Res, 78(19), 5481-5491).

CCNE gene amplification or cyclinE overexpressed forms of cancer cells over activates CDK2, dysregulating Rb phosphorylation and resulting in cancer cell proliferation (Wood, D. J. et al., Cell Chem Biol, 26(1), 121-130). Aberrant CCNE has been proved as a disease driver in multiple cancer types such as ovarian, esophageal, bladder, pancreatic and so on, which are associated with poor disease outcomes (Au-Yeung, G. et al., Clin Cancer Res, 23(7), 1862-1874; DeLair. D. F. et al., J Pathol, 243(2), 230-241; Fu, Y. P. et al., Cancer Res, 74(20), 5808-5818; Huber, A. R. et al., BMC Gastroenterol, 15, 80; Miller, C. T. et al., Clin Cancer Res, 9(13), 4819-4825). Besides, one of the mechanisms for the clinical drug resistance of CDK4/6i (such as Palbociclib, Ribociclib and Abemaciclib) in ER+HER2− breast cancer patients is the overexpression of CCNE and the activation of CDK2 (Knudsen, E. S. et al., Cell Rep, 38(9), 110448). CDK2 siRNA knockdown in the Palbociclib resistant breast cancer cell lines and mouse model shows CDK2 inhibition can overcome the CDK4/6-inhibitor resistance (Pandey, K. et al., Cancers (Basel), 12(12)). Another potential use of CDK2 inhibitors is based on the mechanism of trastuzumab resistance. There is around 35% trastuzumab resistance incidence of cyclinE amplification or overexpression in HER2+BC patients, which are associated with a worse clinical benefit while the trastuzumab-resistant cells are sensitive to CDK2 inhibition (Scaltriti, M. et al., Proc Natl Acad Sci USA, 108(9), 3761-3766). The CCNE1 amplification across broad cancer types predicts response to CDK2 inhibition, indicating CDK2 is a potentially impactful therapeutic target.

The low toxicity of targeting CDK2 has also been proved in the animal model. CDK2 knockout mice are viable and developed normally except for the abnormal germline cell (Berthet, C. et al., Curr Biol, 13(20), 1775-1785). While single knockout of CDK1 or double knockout of CDK4 and CDK6 mice are embryonic lethal (Satyanarayana, A, and Kaldis. P., Oncogene, 28(33), 2925-2939).

The crystal structure of CDK2 especially its kinase domain is highly similar to the CDK1 and the commercial CDK2 inhibitors are mostly with poor CDK1 selectivity (Wells, C. I. et al., Nat Commun. 11(1), 2743). It is desirable to achieve a cancerous specific drug so as to derive a high selective CDK2 inhibitor, sparing CDK family members to limit off-target CDK-driven toxicities, especially CDK1/CDK4/CDK6.

### SUMMARY OF THE INVENTION

The inventors of the instant invention unexpectedly found that the linear alkyl group attached to the rightmost nitrogen atom (at least compared to the counterpart with a branched alkyl group such as isopropyl) show lower toxicity and the compounds of the instant invention potentially cause less adverse effects when acting on human body.

In one embodiment, disclosed herein are bicyclic compound derivatives of Formula (I). The embodiment comprises the following aspects:
Aspect 1. A compound of Formula (I):

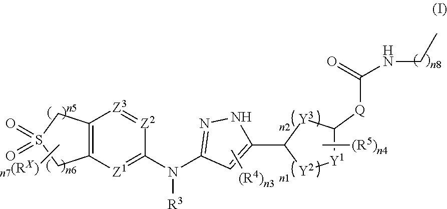

or a N-oxide thereof, or a pharmaceutically acceptable salt thereof, or a stereoisomer thereof, or a tautomer, or a deuterated analog thereof, or a prodrug thereof, wherein:
n1 is 0, 1, or 2;
n2 is 1.2 or 3;
n3 is 0, 1, or 2;
n4 is 0, 1, 2, 3 or 4;
n5 and n6 are each independently 0, 1 or 2, provided that n5 and n6 are not 0 at the same time; n7 is 0, 1, 2, 3 or 4;
n8 is 1, 2, 3, or 4;
R^{X} is each independently selected from hydrogen, halogen, —C₁-C₅alkyl, C₃-C₈cycloalkyl, 3- to 8-membered heterocyclyl, C₆-C₁₂aryl, 5- to 12-membered heteroaryl, oxo (═O), —NR^{Xa}R^{Xb}, —OR^{Xa}, —SR^{Xa}, —SO₂R^{Xa}, —SO₂NR^{Xa}R^{Xb}, —C(O)R^{Xa}, —CO₂R^{Xa}, —C(O)NR^{Xa}R^{Xb}, —NR^{Xa}COR^{Xb}, —NR^{Xa}CO₂R^{Xb} or —NR^{Xa}SO₂R^{Xb} or —CN, wherein each of said —C₁-C₈alkyl, C₃-C₈cycloalkyl, 3- to 8-membered heterocyclyl, C₆-C₁₂aryl, or 5- to 12-membered heteroaryl is optionally substituted with at least one R^{Xc}; or
two R^{X} together with the atom(s) to which they are attached, form a 3- to 12-membered ring, said ring comprising 0-3 heteroatoms independently selected from nitrogen, oxygen or optionally oxidized sulfur as ring member(s); said ring is optionally substituted with at least one substituent R^{Xc};
R^{Xa} and R^{Xb} are each independently selected from hydrogen, deuterium, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl, wherein each of said —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl is optionally substituted with at least one substituent R^{Xd};
R^{Xc} and R^{Xd} are each independently selected from hydrogen, deuterium, halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl. C₆-C₁₂aryl, 5- to 12-membered heteroaryl, oxo (═O), —NR^{Xe}R^{Xf}, —OR^{Xe}, —SR^{Xe}, —SO₂R^{Xe}, —SO₂NR^{Xe}R^{Xf}, —C(O)R^{Xe}, —CO₂R^{Xe}, —C(O)NR^{Xe}R^{Xf}, —NR^{Xe}COR^{Xf}, —NR^{Xe}CO₂R^{Xf} or —NR^{Xe}SO₂R^{Xf} or —CN; R^{Xe} and R^{Xf} are each independently selected from —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;
Y¹, Y² and Y³ are each independently selected from O, CH₂ or NH;
Q is selected from O or NR^{Q};
R³ and R^{Q} are each independently selected from hydrogen, —C₁-C₈alkyl, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl, wherein each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{3a}; or
R^{3a} is independently selected from hydrogen, halogen, —C₁-C₈alkyl, —C₂-C₈alkenyl, —C₂-C₈alkynyl, —C₃-C₈cycloalkyl, 3- to 8-membered heterocyclyl, —C₆-C₁₂aryl, 5- to 12-membered heteroaryl, oxo (═O), —OR^{3c}, —CN, —SO₂R^{3c}, —SO₂NR^{3c}R^{3d}, —C(O)R^{3c}, —CO₂R^{3c}, —C(O)NR^{3c}R^{3d}, —NR^{3c}R^{3d}, —NR^{3c}COR^{3d}, —NR^{3c}CO₂R^{3d} or —NR^{3c}SO₂R^{3d}, wherein each of —C₁-C₈alkyl, —C₂-C₈alkenyl, —C₂-C₈alkynyl, —C₁-C₈alkoxy, —C₃-C₈cycloalkyl, 3- to 8-membered heterocyclyl, —C₆-C₁₂aryl or 5- to 12-membered heteroaryl is optionally substituted with halogen, —C₁-C₈alkyl, —C₂-C₈alkenyl, —C₂-C₈alkynyl, —C₃-C₈cycloalkyl, 3- to 8-membered heterocyclyl, C₆-C₁₂aryl, 5- to 12-membered heteroaryl, oxo (═O), —CN, —OR^{3e}, —SO₂R^{3e}, —SO₂NR^{3e}R^{3f}, —C(O)R^{3e}, —CO₂R^{3f}, —C(O)NR^{3e}R^{3f}, —NR^{3e}R^{3f}, —NR^{3e}COR^{3f}, —NR^{3e}CO₂R^{3f} or —NR^{3e}SO₂R^{3f};
R^{3c}, R^{3d}, R^{3e} and R^{3f} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;
R⁴ and R⁵ are each independently selected from hydrogen, halogen, —C₁-C₈alkyl, —C₂-C₈alkenyl, —C₂-C₈alkynyl, —C₁-C₈alkoxy, or —C₃-C₈cycloalkyl;
Z¹, Z² and Z³ are each independently selected from —CR^{Z}, or N;
R^{Z}, at each occurrence, is independently selected from hydrogen, halogen, —C₁-C₈alkyl, C₃-C₈cycloalkyl, 3- to 8-membered heterocyclyl, C₆-C₁₂aryl, 5- to 12-membered heteroaryl, —NR^{Za}R^{Zb}, —OR^{Za}, —SR^{Za}, —SO₂R^{Za}, —SO₂NR^{Za}R^{Zb}, —C(O)R^{Za}, —CO₂R^{Za}, —C(O)NR^{Za}R^{Zb}, —NR^{Za}COR^{Zb}, —NR^{Za}CO₂R^{Zb} or —NR^{za}SO₂R^{Zb} or —CN, wherein each of said —C₁-C₈alkyl, C₃-C₈cycloalkyl, 3- to 8-membered heterocyclyl, C₆-C₁₂aryl, or 5- to 12-membered heteroaryl is optionally substituted with at least one R^{Zc};
R^{Za} and R^{Zb} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl, wherein each of said —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl is optionally substituted with at least one substituent R^{Zd};
R^{Zc} and R^{Zd} are each independently selected from halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl, 5- to 12-membered heteroaryl, oxo (═O), —NR^{Ze}R^{Zf}, —OR^{Ze}, —SR^{Ze}, —SO₂R^{Ze}, —SO₂NR^{Ze}R^{Zf}, —C(O)R^{Ze}, —CO₂R^{Ze}, —C(O)NR^{Ze}R^{Zf}, —NR^{Ze}COR^{Zf}, —NR^{Ze}CO₂R^{Zf} or —NR^{ze}SO₂R^{Zf} or —CN;
R^{Ze} and R^{Zf} are each independently selected from —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl.
Aspect 2. The compound of Aspect 1, wherein the compound is selected from (IIa) and (IIb):

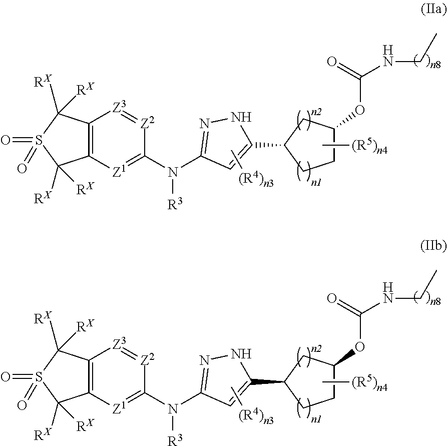

preferably, the compound is (IIIa):

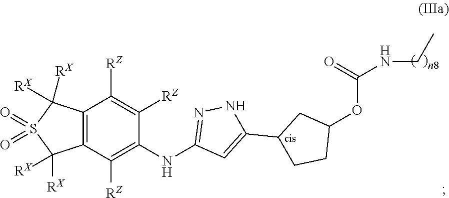

preferably, the compound is selected from (IVa) and (IVb):

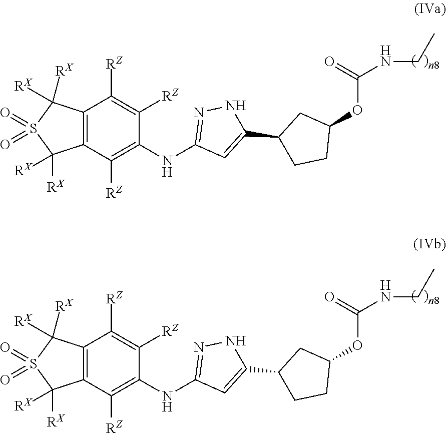

Aspect 3. The compound of any one of the preceding aspects, wherein R³ and R^{Q} are each independently selected from hydrogen, methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, wherein each of said methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{3a};
R^{3a} is independently selected from hydrogen, —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —OR^{3c}, —CN, —SO₂R^{3c}, —SO₂NR^{3c}R^{3d}, —C(O)R^{3c}, —CO₂R^{3c}, —C(O)NR^{3c}R^{3d}, —NR^{3c}R^{3d}, —NR^{3c}COR^{3d}, —NR^{3c}CO₂R^{3d} or —NR^{3c}SO₂R^{3d}, wherein each of said methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl, 3- to 8-membered heterocyclyl, phenyl, 5- to 12-membered heteroaryl is optionally substituted with —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —CN, —OR^{3e}, —SO₂R^{3e}, —SO₂NR^{3e}R^{3f}, —C(O)R^{3e}, —CO₂R^{3e}, —C(O)NR^{3e}R^{3f}, —NR^{3e}R^{3f}, —NR^{3e}COR^{3f}, —NR^{3e}CO₂R^{3f} or —NR^{3e}SO₂R^{3f};
R^{3c}, R^{3d}, R^{3e} and R^{3f} are each independently selected from hydrogen, methyl, ethyl, propyl (n-propyl or isopropyl), butyl (n-butyl, sec-butyl, iso-butyl or tert-butyl), pentyl, hexyl, heptyl, octyl, —C₂-C₈alkenyl. —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl, wherein each of said methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —CN, —OH, methoxy, ethoxy, propoxy, butoxy, pentoxy, hexoxy, heptoxy, or octoxy;
preferably, R³ and R^{Q} are each independently selected from hydrogen, methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
more preferably, R³ and R^{Q} are each independently selected from hydrogen, methyl, ethyl, propyl (n-propyl or isopropyl), butyl (n-butyl, sec-butyl, iso-butyl or tert-butyl); and even more preferably, R³ and R^{Q} are each independently hydrogen.
Aspect 4. The compound of any one of the preceding aspects, wherein R⁴ and R⁵ are each independently selected from hydrogen, —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl;
preferably, R⁴ and R⁵ are each independently selected from hydrogen, —F, —Cl, —Br, —I, methyl, ethyl, propyl (n-propyl or isopropyl), butyl (n-butyl, sec-butyl, iso-butyl or tert-butyl), methoxy, ethoxy, propoxy, cyclopropyl, cyclobutyl, cyclopentyl;
even more preferably, R⁴ and R⁵ are each independently selected from hydrogen.
Aspect 5. The compound of any one of the preceding aspects, wherein the

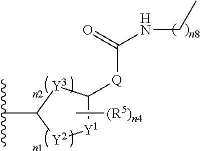

moiety is selected from

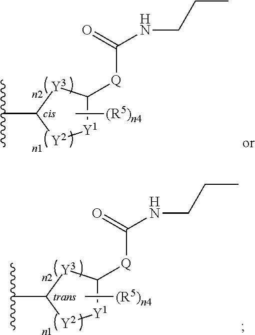

preferably, the

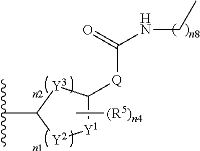

moiety is selected from

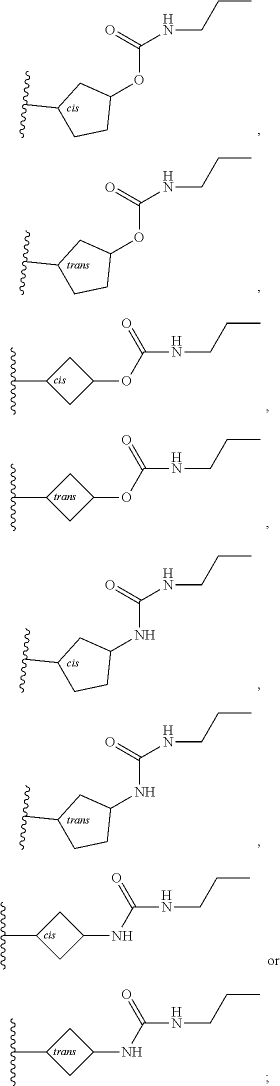

more preferably, the

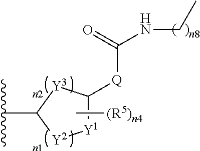

moiety is selected from

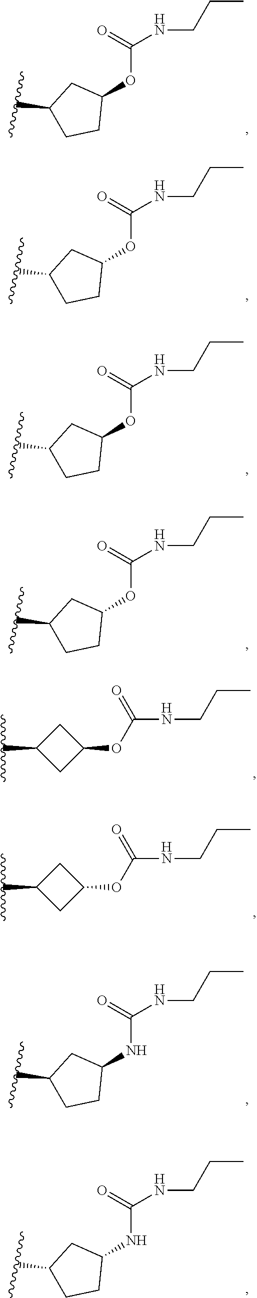

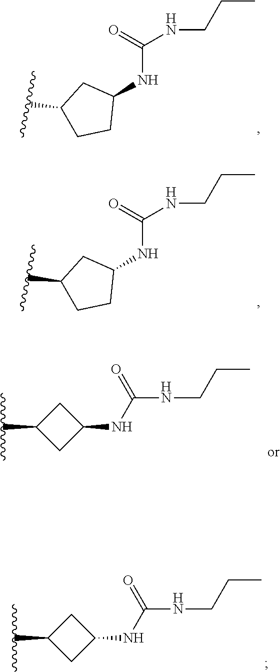 [SMILES: *[C@H]1CC[C@H](NC(=O)NCCC)C1 ; *[C@@H]1CC[C@@H](NC(=O)NCCC)C1 ; *[C@H]1C[C@@H](NC(=O)NCCC)C1 ; *[C@H]1C[C@H](NC(=O)NCCC)C1]

even more preferably, the

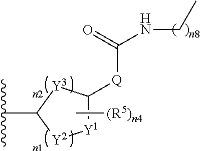

moiety is selected from

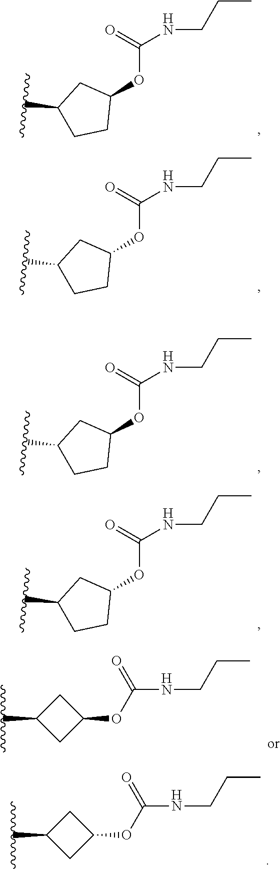

Aspect 6. The compound of any one of the preceding aspects, wherein at most two of Z¹, Z² and Z³ are N; preferably, at most one of Z¹, Z² and Z³ is N;
more preferably, Z¹, Z² and Z³ are independently —CR^{Z}.
Aspect 7. The compound of any one of the preceding aspects, wherein R^{Z}, at each occurrence, is independently selected from hydrogen, —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —NR^{Za}R^{Zb}, —OR^{Za}, —SR^{Za}, —SO₂R^{Za}, —SO₂NR^{Za}R^{Zb}, —C(O)R^{Za}, —CO₂R^{Za}, —C(O)NR^{Za}R^{Zb}, —NR^{Za}COR^{Zb}, —NR^{Za}CO₂R^{Zb} or —NR^{Za}SO₂R^{Zb} or —CN, wherein each of said methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one R^{Zc};
R^{Za} and R^{Zb} are each independently selected from hydrogen, methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl, wherein each of said methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one substituent R^{Zd};
R^{Zc} and R^{Zd} are each independently selected from —F, —Cl, —Br, —I, hydroxy, methyl, ethyl, propyl (n-propyl or isopropyl), butyl (n-butyl, sec-butyl, iso-butyl or tert-butyl), pentyl, hexyl, heptyl, octyl, methoxy, ethoxy, propoxy, butoxy, pentoxy, hexoxy, heptoxy, octoxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —NR^{Ze}R^{Zf}, —OR^{Ze}, —SR^{Ze}, —SO₂R^{Ze}, —SO₂NR^{Ze}R^{Zf}, —C(O)R^{Ze}, —CO₂R^{Ze}, —C(O)NR^{Ze}R^{Zf}, —NR^{Ze}COR^{Zf}, —NR^{Ze}CO₂R^{Zf} or —NR^{Ze}SO₂R^{Zf} or —CN;
R^{Ze} and R^{Zf} are each independently selected from methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl;
preferably, R^{Z}, at each occurrence, is independently selected from hydrogen, —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —CH₂F, —CHF₂, —CF₃, —CH₂CH₂F, —CH₂CHF₂, —CH₂CF₃, —NR^{Za}R^{Zb}, —OR^{Za}, —SR^{Za}, —SO₂R^{Za}, —SO₂NR^{Za}R^{Zb}, —C(O)R^{Za}, —CO₂R^{Za}, —C(O)NR^{Za}R^{Zb}, —NR^{Za}COR^{Zb}, —NR^{Za}CO₂R^{Zb} or —NR^{Za}SO₂R^{Zb} or —CN;
R^{Za} and R^{Zb} are each independently selected from hydrogen, methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
more preferably, R^{Z}, at each occurrence, is independently selected from hydrogen, —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —CH₂F, —CHF₂, —CF₃, —CH₂CH₂F, —CH₂CHF₂, —CH₂CF₃, —NH₂, —OH, methoxy, ethoxy, propoxy, butoxy, pentoxy, hexoxy, heptoxy, octoxy or —CN; and
even more preferably, R^{Z}, at each occurrence, is independently selected from hydrogen, —F, and methyl.
Aspect 8. The compound of any one of the preceding aspects, wherein the moiety is selected

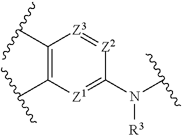

from

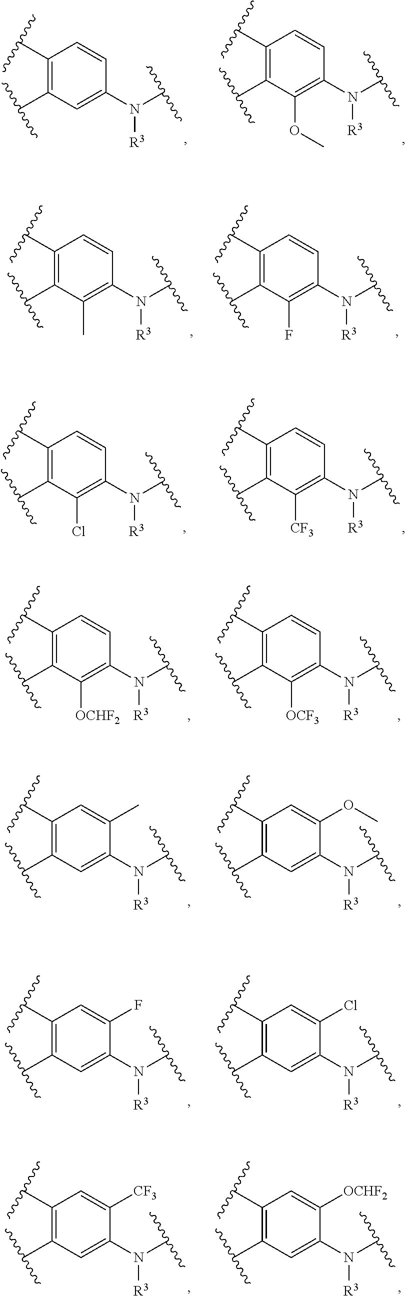

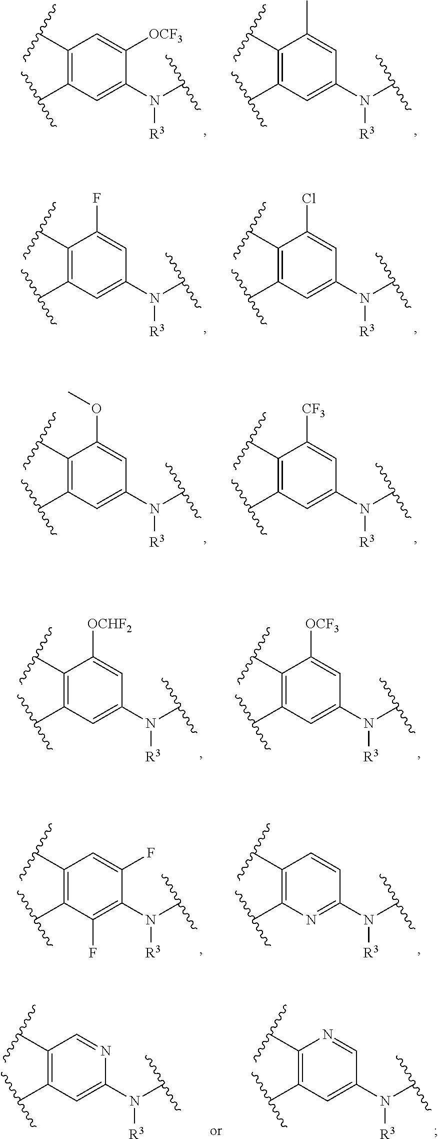

preferably, the

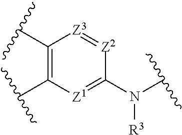

moiety is selected from

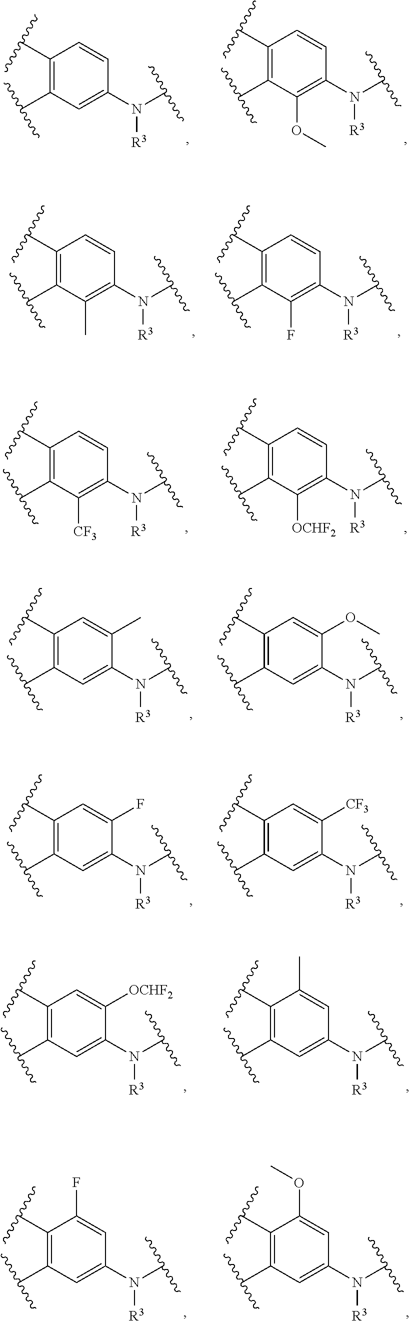

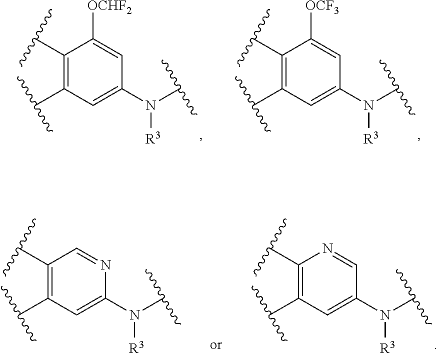

Aspect 9. The compound of any one of the preceding aspects, wherein R^{X} is each independently selected from hydrogen, —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —NR^{Xa}R^{Xb}, —OR^{Xa}, —SR^{Xa}, —SO₂R^{Xa}, —SO₂NR^{Xa}R^{Xb}, —C(O)R^{Xa}, —CO₂R^{Xa}, —C(O)NR^{Xa}R^{Xb}, —NR^{Xa}COR^{Xb}, —NR^{Xa}CO₂R^{Xb} or —NR^{Xa}SOR^{Xb} or —CN, wherein each of said methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one R^{Xc};
R^{Xa} and R^{Xb} are each independently selected from hydrogen, methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl, wherein each of said methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one substituent R^{Xd};
R^{Xc} and R^{Xd} are each independently selected from —F, —Cl, —Br, —I, hydroxy, methyl, ethyl, propyl (n-propyl or isopropyl), butyl (n-butyl, sec-butyl, iso-butyl or tert-butyl), pentyl, hexyl, heptyl, octyl, methoxy, ethoxy, propoxy, butoxy, pentoxy, hexoxy, heptoxy, octoxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —NR^{Xe}R^{Xf}, —OR^{Xe}, —SR^{Xe}, —SO₂R^{Xe}, —SO₂NR^{Xe}R^{Xf}, —C(O)R^{Xe}, —CO₂R^{Xe}, —C(O)NR^{Xe}R^{Xf}, —NR^{Xe}COR^{Xf}, —NR^{Xe}CO₂R^{Xf} or —NR^{Xe}SO₂R^{Xf} or —CN;
R^{Xe} and R^{Xf} are each independently selected from methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl;
preferably, R^{X} is independently selected from hydrogen, —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —CH₂F, —CHF₂, —CF₃, —CH₂CH₂F, —CH₂CHF₂, —CH₂CF₃, oxo (═O), —NR^{Xa}R^{Xb}, —OR^{Xa}, —SR^{Xa}, —SO₂R^{Xa}, —SO₂NR^{Xa}R^{Xb}, —C(O)R^{Xa}, —CO₂R^{Xa}, —C(O)NR^{Xa}R^{Xb}, —NR^{Xa}COR^{Xb}, —NR^{Xa}CO₂R^{Xb} or —NR^{Xa}SO₂R^{Xb} or —CN;
R^{Xa} and R^{Xb} are each independently selected from hydrogen, methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
more preferably, R^{X} is independently selected from hydrogen, —F, —Cl, methyl, ethyl, propyl (n-propyl or isopropyl), butyl (n-butyl, sec-butyl, iso-butyl or tert-butyl), cyclopropyl, cyclobutyl, cyclopentyl, cyclohexyl, —CH₂F, —CHF₂, —CF₃, —CH₂CH₂F, —CH₂CHF₂, —CH₂CF₃, oxo (═O), or —OH; and
even more preferably, R^{X} is hydrogen.
Aspect 10. The compound of any one of the preceding aspects, wherein two R^{X} together with the atom(s) to which they are attached, form a 3-, 4-, 5-, 6-, 7- or 8-membered ring, said ring comprising 0, 1, 2 or 3 heteroatoms independently selected from nitrogen, oxygen or optionally oxidized sulfur as ring member(s); said ring is optionally substituted with at least one substituent R^{Xc};
R^{Xc} is each independently selected from —F, —Cl, —Br, —I, hydroxy, methyl, ethyl, propyl (n-propyl or isopropyl), butyl (n-butyl, sec-butyl, iso-butyl or tert-butyl), pentyl, hexyl, heptyl, octyl, methoxy, ethoxy, propoxy, butoxy, pentoxy, hexoxy, heptoxy, octoxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —NR^{Xe}R^{Xf}, —OR^{Xe}, —SR^{Xe}, —SO₂R^{Xe}, —SO₂NR^{Xe}R^{Xf}, —C(O)R^{Xe}, —CO₂R^{Xe}, —C(O)NR^{Xe}R^{Xf}, —NR^{Xe}COR^{Xf}, —NR^{Xe}CO₂R^{Xf} or —NR^{Xe}SO₂R^{Xf} or —CN;
R^{Xe} and R^{Xf} are each independently selected from methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl;
preferably, two R^{X} together with the atom(s) to which they are attached, form a 3-, 4-, 5- or 6-membered ring; said ring is optionally substituted with at least one substituent R^{Xc};
R^{Xc} is each independently selected from —F, —Cl, —Br, —I, hydroxy, methyl, ethyl, propyl (n-propyl or isopropyl), butyl (n-butyl, sec-butyl, iso-butyl or tert-butyl), methoxy, ethoxy, propoxy, butoxy, cyclopropyl, cyclobutyl, cyclopentyl, cyclohexyl, oxo (═O) or —CN;
more preferably, two R^{X} together with the atom(s) to which they are attached, form a 3-, 4-, 5- or 6-membered ring.
Aspect 11. The compound of any one of the preceding aspects, wherein the

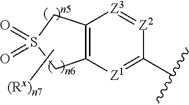

moiety is selected from

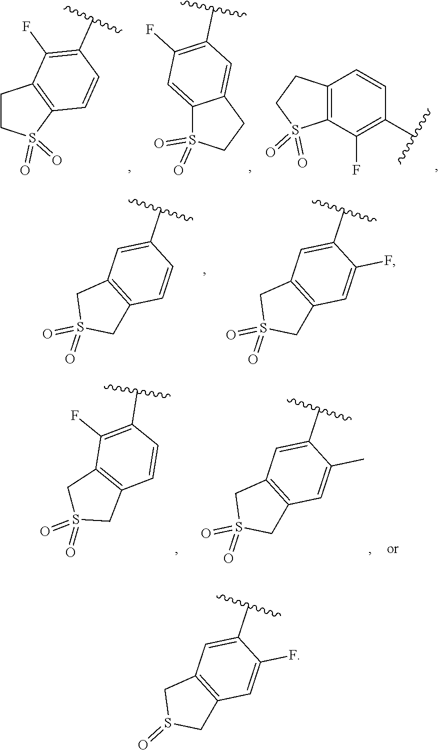

Aspect 12. A compound, or a N-oxide thereof, or a pharmaceutically acceptable salt thereof, or a stereoisomer thereof, or a tautomer, or a deuterated analog thereof, or a prodrug thereof, wherein the compound is selected from

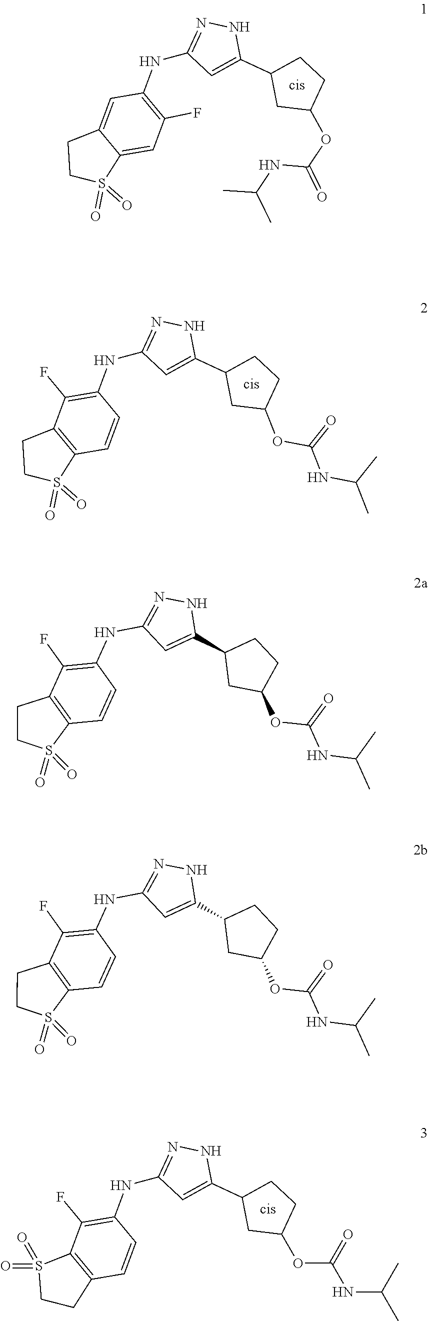 [SMILES: CC(C)NC(=O)OC1CCC(c2cc(Nc3cc4c(cc3F)S(=O)(=O)CC4)n[nH]2)C1 ; CC(C)NC(=O)OC1CCC(c2cc(Nc3ccc4c(c3F)CCS4(=O)=O)n[nH]2)C1 ; CC(C)NC(=O)O[C@@H]1CC[C@H](c2cc(Nc3ccc4c(c3F)CCS4(=O)=O)n[nH]2)C1 ; CC(C)NC(=O)O[C@H]1CC[C@@H](c2cc(Nc3ccc4c(c3F)CCS4(=O)=O)n[nH]2)C1 ; CC(C)NC(=O)OC1CCC(c2cc(Nc3ccc4c(c3F)S(=O)(=O)CC4)n[nH]2)C1]

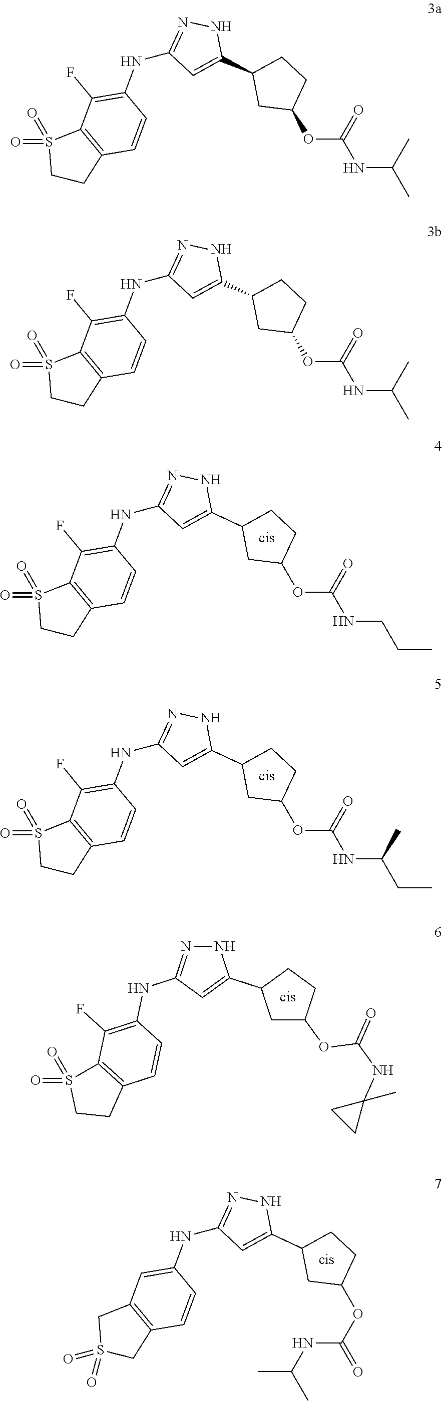 [SMILES: CC(C)NC(=O)O[C@@H]1CC[C@H](c2cc(Nc3ccc4c(c3F)S(=O)(=O)CC4)n[nH]2)C1 ; CC(C)NC(=O)O[C@H]1CC[C@@H](c2cc(Nc3ccc4c(c3F)S(=O)(=O)CC4)n[nH]2)C1 ; CCCNC(=O)OC1CCC(c2cc(Nc3ccc4c(c3F)S(=O)(=O)CC4)n[nH]2)C1 ; CC[C@H](C)NC(=O)OC1CCC(c2cc(Nc3ccc4c(c3F)S(=O)(=O)CC4)n[nH]2)C1 ; CC1(NC(=O)OC2CCC(c3cc(Nc4ccc5c(c4F)S(=O)(=O)CC5)n[nH]3)C2)CC1 ; CC(C)NC(=O)OC1CCC(c2cc(Nc3ccc4c(c3)CS(=O)(=O)C4)n[nH]2)C1]

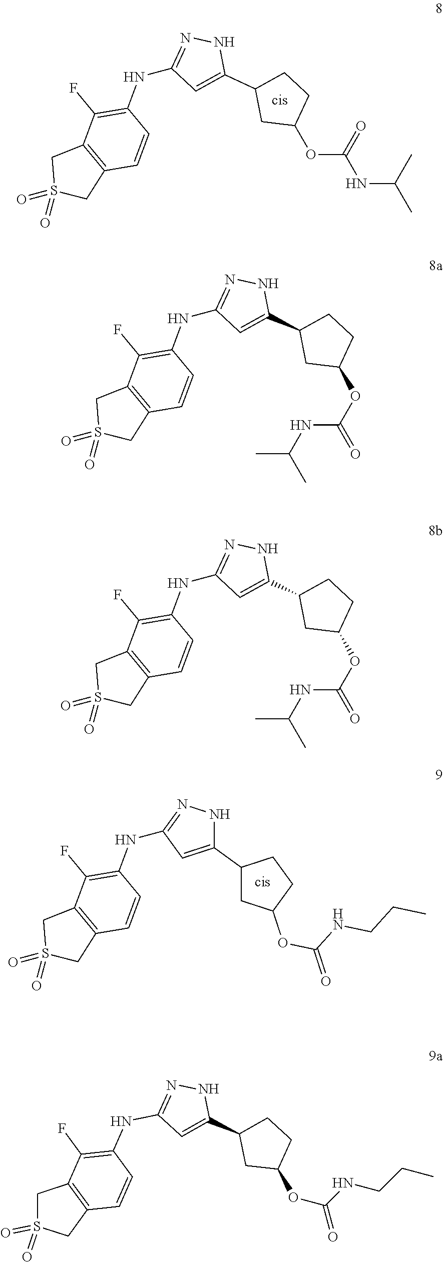 [SMILES: CC(C)NC(=O)OC1CCC(c2cc(Nc3ccc4c(c3F)CS(=O)(=O)C4)n[nH]2)C1 ; CC(C)NC(=O)O[C@@H]1CC[C@H](c2cc(Nc3ccc4c(c3F)CS(=O)(=O)C4)n[nH]2)C1 ; CC(C)NC(=O)O[C@H]1CC[C@@H](c2cc(Nc3ccc4c(c3F)CS(=O)(=O)C4)n[nH]2)C1 ; CCCNC(=O)OC1CCC(c2cc(Nc3ccc4c(c3F)CS(=O)(=O)C4)n[nH]2)C1 ; CCCNC(=O)O[C@@H]1CC[C@H](c2cc(Nc3ccc4c(c3F)CS(=O)(=O)C4)n[nH]2)C1]

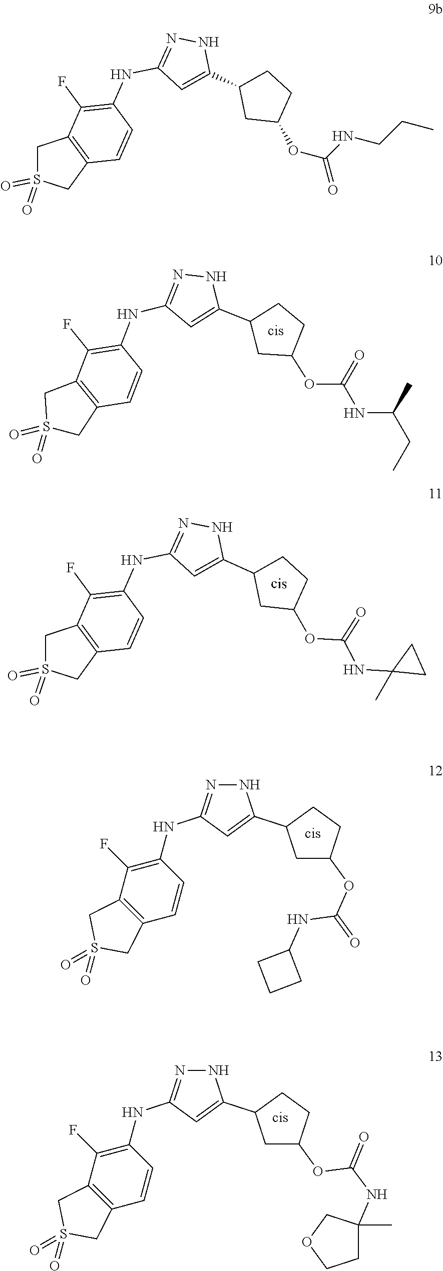 [SMILES: CCCNC(=O)O[C@H]1CC[C@@H](c2cc(Nc3ccc4c(c3F)CS(=O)(=O)C4)n[nH]2)C1 ; CC[C@H](C)NC(=O)OC1CCC(c2cc(Nc3ccc4c(c3F)CS(=O)(=O)C4)n[nH]2)C1 ; CC1(NC(=O)OC2CCC(c3cc(Nc4ccc5c(c4F)CS(=O)(=O)C5)n[nH]3)C2)CC1 ; O=C(NC1CCC1)OC1CCC(c2cc(Nc3ccc4c(c3F)CS(=O)(=O)C4)n[nH]2)C1 ; CC1(NC(=O)OC2CCC(c3cc(Nc4ccc5c(c4F)CS(=O)(=O)C5)n[nH]3)C2)CCOC1]

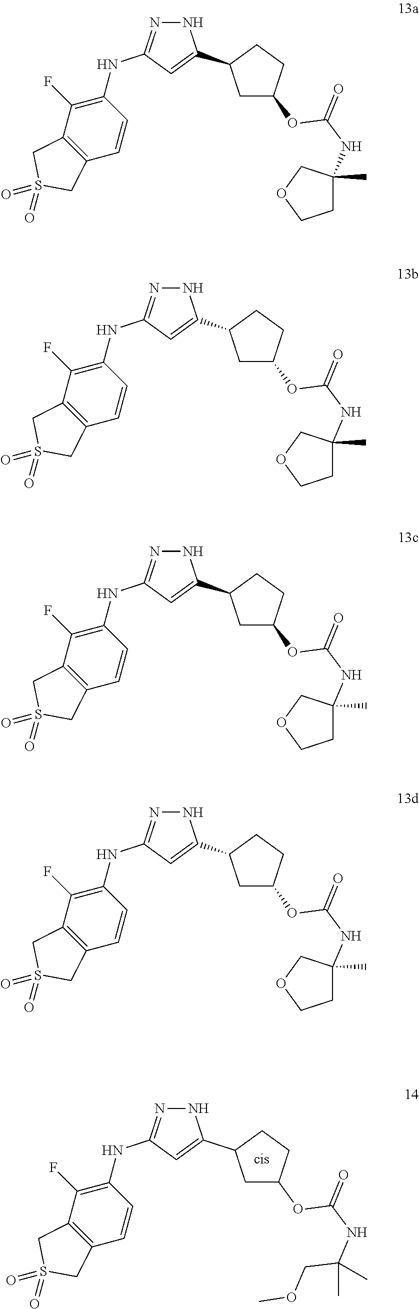 [SMILES: C[C@@]1(NC(=O)O[C@@H]2CC[C@H](c3cc(Nc4ccc5c(c4F)CS(=O)(=O)C5)n[nH]3)C2)CCOC1 ; C[C@@]1(NC(=O)O[C@H]2CC[C@@H](c3cc(Nc4ccc5c(c4F)CS(=O)(=O)C5)n[nH]3)C2)CCOC1 ; C[C@]1(NC(=O)O[C@@H]2CC[C@H](c3cc(Nc4ccc5c(c4F)CS(=O)(=O)C5)n[nH]3)C2)CCOC1 ; C[C@]1(NC(=O)O[C@H]2CC[C@@H](c3cc(Nc4ccc5c(c4F)CS(=O)(=O)C5)n[nH]3)C2)CCOC1 ; COCC(C)(C)NC(=O)OC1CCC(c2cc(Nc3ccc4c(c3F)CS(=O)(=O)C4)n[nH]2)C1]

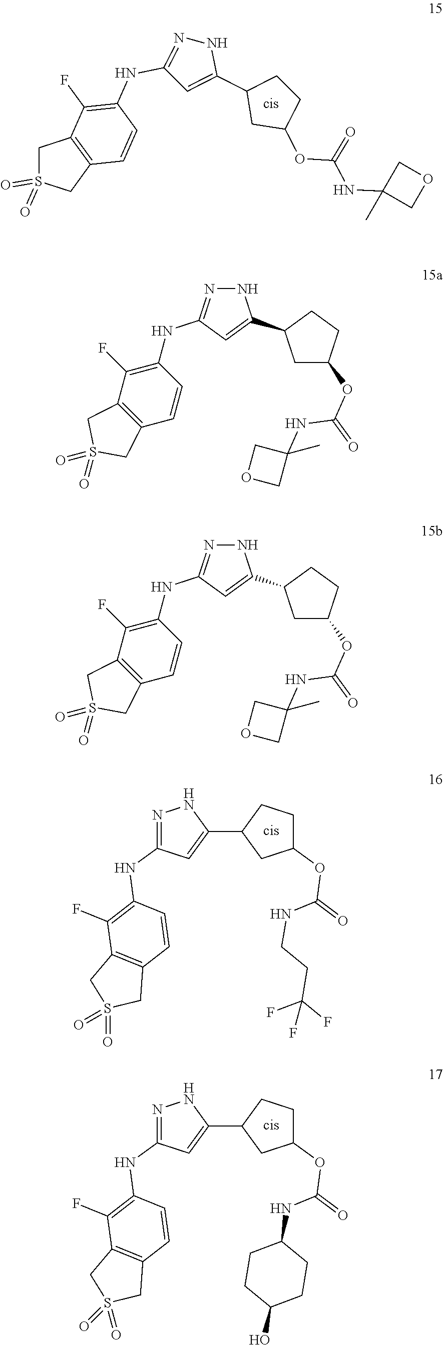 [SMILES: CC1(NC(=O)OC2CCC(c3cc(Nc4ccc5c(c4F)CS(=O)(=O)C5)n[nH]3)C2)COC1 ; CC1(NC(=O)O[C@@H]2CC[C@H](c3cc(Nc4ccc5c(c4F)CS(=O)(=O)C5)n[nH]3)C2)COC1 ; CC1(NC(=O)O[C@H]2CC[C@@H](c3cc(Nc4ccc5c(c4F)CS(=O)(=O)C5)n[nH]3)C2)COC1 ; O=C(NCCC(F)(F)F)OC1CCC(c2cc(Nc3ccc4c(c3F)CS(=O)(=O)C4)n[nH]2)C1 ; O=C(N[C@H]1CC[C@@H](O)CC1)OC1CCC(c2cc(Nc3ccc4c(c3F)CS(=O)(=O)C4)n[nH]2)C1]

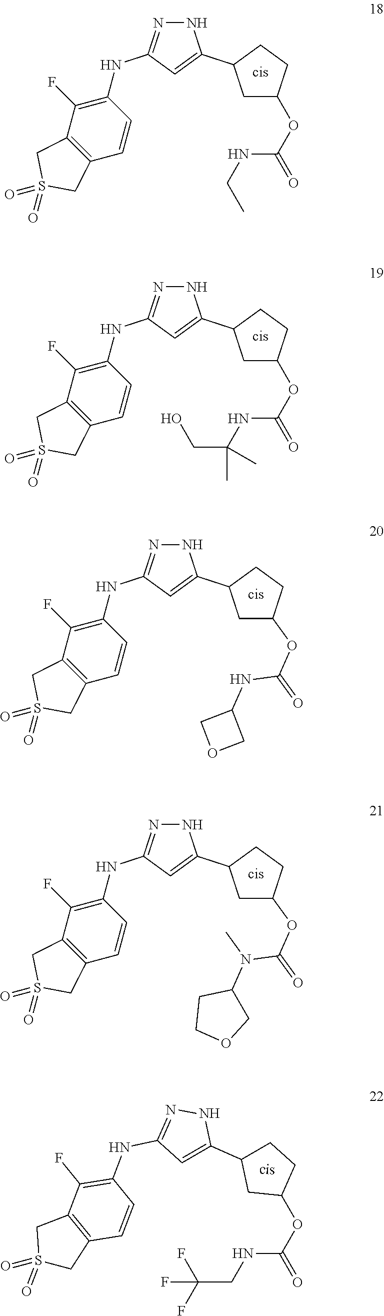 [SMILES: CCNC(=O)OC1CCC(c2cc(Nc3ccc4c(c3F)CS(=O)(=O)C4)n[nH]2)C1 ; CC(C)(CO)NC(=O)OC1CCC(c2cc(Nc3ccc4c(c3F)CS(=O)(=O)C4)n[nH]2)C1 ; O=C(NC1COC1)OC1CCC(c2cc(Nc3ccc4c(c3F)CS(=O)(=O)C4)n[nH]2)C1 ; CN(C(=O)OC1CCC(c2cc(Nc3ccc4c(c3F)CS(=O)(=O)C4)n[nH]2)C1)C1CCOC1 ; O=C(NCC(F)(F)F)OC1CCC(c2cc(Nc3ccc4c(c3F)CS(=O)(=O)C4)n[nH]2)C1]

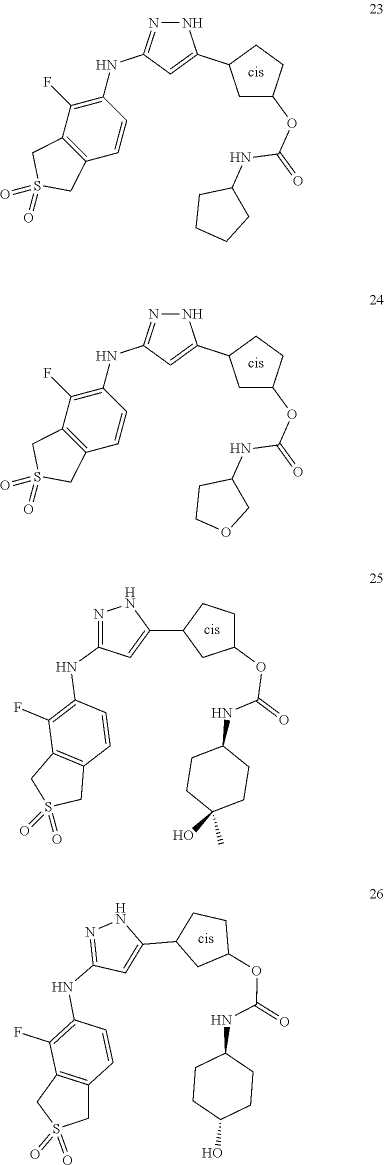 [SMILES: O=C(NC1CCCC1)OC1CCC(c2cc(Nc3ccc4c(c3F)CS(=O)(=O)C4)n[nH]2)C1 ; O=C(NC1CCOC1)OC1CCC(c2cc(Nc3ccc4c(c3F)CS(=O)(=O)C4)n[nH]2)C1 ; C[C@]1(O)CC[C@@H](NC(=O)OC2CCC(c3cc(Nc4ccc5c(c4F)CS(=O)(=O)C5)n[nH]3)C2)CC1 ; O=C(N[C@H]1CC[C@H](O)CC1)OC1CCC(c2cc(Nc3ccc4c(c3F)CS(=O)(=O)C4)n[nH]2)C1]

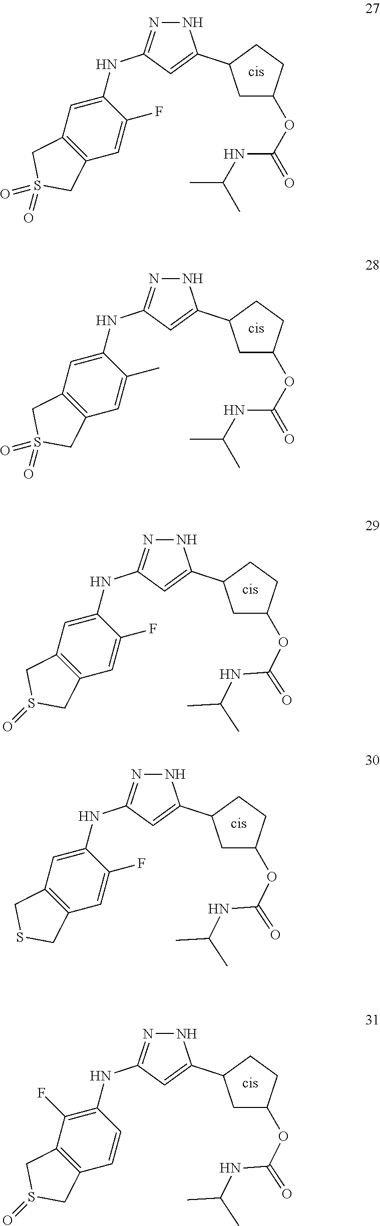 [SMILES: CC(C)NC(=O)OC1CCC(c2cc(Nc3cc4c(cc3F)CS(=O)(=O)C4)n[nH]2)C1 ; Cc1cc2c(cc1Nc1cc(C3CCC(OC(=O)NC(C)C)C3)[nH]n1)CS(=O)(=O)C2 ; CC(C)NC(=O)OC1CCC(c2cc(Nc3cc4c(cc3F)CS(=O)C4)n[nH]2)C1 ; CC(C)NC(=O)OC1CCC(c2cc(Nc3cc4c(cc3F)CSC4)n[nH]2)C1 ; CC(C)NC(=O)OC1CCC(c2cc(Nc3ccc4c(c3F)CS(=O)C4)n[nH]2)C1]

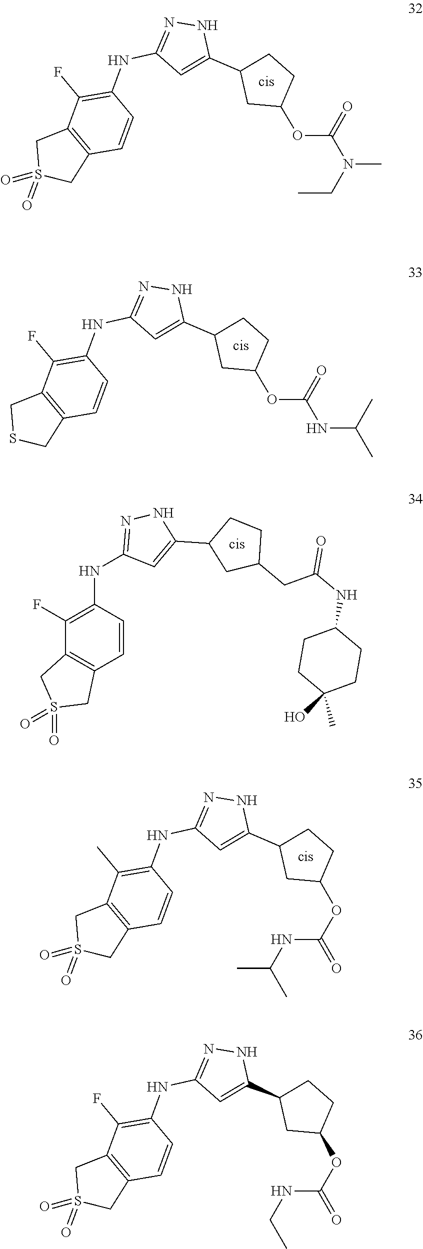 [SMILES: CCN(C)C(=O)OC1CCC(c2cc(Nc3ccc4c(c3F)CS(=O)(=O)C4)n[nH]2)C1 ; CC(C)NC(=O)OC1CCC(c2cc(Nc3ccc4c(c3F)CSC4)n[nH]2)C1 ; C[C@]1(O)CC[C@H](NC(=O)CC2CCC(c3cc(Nc4ccc5c(c4F)CS(=O)(=O)C5)n[nH]3)C2)CC1 ; Cc1c(Nc2cc(C3CCC(OC(=O)NC(C)C)C3)[nH]n2)ccc2c1CS(=O)(=O)C2 ; CCNC(=O)O[C@@H]1CC[C@H](c2cc(Nc3ccc4c(c3F)CS(=O)(=O)C4)n[nH]2)C1]

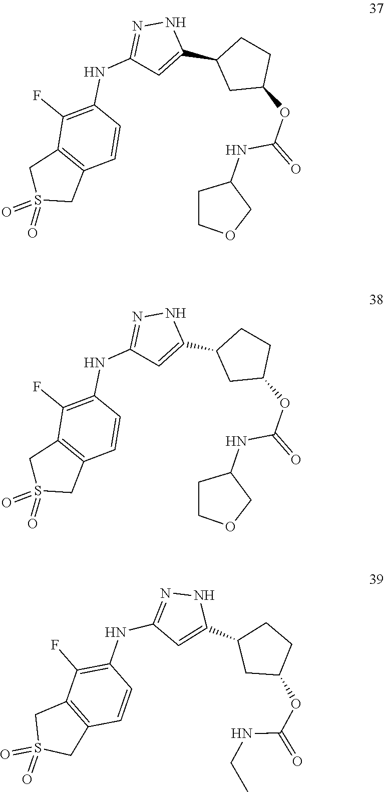 [SMILES: O=C(NC1CCOC1)O[C@@H]1CC[C@H](c2cc(Nc3ccc4c(c3F)CS(=O)(=O)C4)n[nH]2)C1 ; O=C(NC1CCOC1)O[C@H]1CC[C@@H](c2cc(Nc3ccc4c(c3F)CS(=O)(=O)C4)n[nH]2)C1 ; CCNC(=O)O[C@H]1CC[C@@H](c2cc(Nc3ccc4c(c3F)CS(=O)(=O)C4)n[nH]2)C1]

Aspect 13. A pharmaceutical composition comprising a compound of any one of aspects 1-12, or a pharmaceutically acceptable salt thereof, or a stereoisomer, a tautomer or a prodrug thereof, and at least one pharmaceutically acceptable carrier or excipient.
Aspect 14. A method of treating cancer, comprising administering to a subject in need thereof a compound of any one of aspects 1-12, or a pharmaceutically acceptable salt, or a stereoisomer, a tautomer or a prodrug thereof.

### DETAILED DESCRIPTION OF THE INVENTION

The following terms have the indicated meanings throughout the specification:

As used herein, including the appended Aspects, the singular forms of words such as “a”, “an”, and “the”, include their corresponding plural references unless the context clearly dictates otherwise.

The term “or” is used to mean, and is used interchangeably with, the term “and/or” unless the context clearly dictates otherwise.

The term “alkyl” refers to a hydrocarbon group selected from linear and branched saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “cycloalkyl” refers to a hydrocarbon group selected from saturated cyclic hydrocarbon groups, comprising monocyclic and polycyclic (e.g., bicyclic and tricyclic) groups including fused, bridged or spiro cycloalkyl.

The term “aryl” used alone or in combination with other terms refers to a group selected from:
5- and 6-membered carbocyclic aromatic rings, e.g., phenyl;
bicyclic ring systems such as 7- to 12-membered bicyclic ring systems, wherein at least one ring is carbocyclic and aromatic, e.g., naphthyl and indanyl; and,
tricyclic ring systems such as 10- to 15-membered tricyclic ring systems wherein at least one ring is carbocyclic and aromatic, e.g., fluorenyl.

The terms “aromatic hydrocarbon ring” and “aryl” are used interchangeable throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring include, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

The term “aryl-alkyl-” refers to an alkyl group as defined above which is further substituted by an aryl group. Examples of an aryl-alkyl group include aryl-C₁₋₈alkyl, such as phenylethyl, or phenylmethyl (benzyl).

The term “heteroaryl” refers to a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides. The term “C-linked heteroaryl” as used herein means that the heteroaryl group is connected to the core molecule by a bond from a C-atom of the heteroaryl ring

The terms “aromatic heterocyclic ring” and “heteroaryl” are used interchangeable throughout the disclosure herein. In some embodiments, a monocyclic or bicyclic aromatic heterocyclic ring has 5-, 6-, 7-, 8-, 9- or 10-ring forming members with 1, 2, 3, or 4 heteroatom ring members independently selected from nitrogen (N), sulfur (S) and oxygen (O) and the remaining ring members being carbon. In some embodiments, the monocyclic or bicyclic aromatic heterocyclic ring is a monocyclic or bicyclic ring comprising 1 or 2 heteroatom ring members independently selected from nitrogen (N), sulfur (S) and oxygen (O). In some embodiments, the monocyclic or bicyclic aromatic heterocyclic ring is a 5- to 6-membered heteroaryl ring, which is monocyclic and which has 1 or 2 heteroatom ring members independently selected from nitrogen (N), sulfur (S) and oxygen (O). In some embodiments, the monocyclic or bicyclic aromatic heterocyclic ring is an 8- to 10-membered heteroaryl ring, which is bicyclic and which has 1 or 2 heteroatom ring members independently selected from nitrogen, sulfur and oxygen.

“Heterocyclyl”, “heterocycle” or “heterocyclic” are interchangeable and refer to a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups. The term “optionally oxidized sulfur” used herein refers to S, SO or SO₂.

“Hydrogen” and “H” are interchangeable and refer to anyone of protium (¹H), deuterium (²H) or tritium (³H). “Deuterated analog” refers to one or more protiums (¹H) of the compound are substituted with equal numbers of deuteriums (²H).

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included.

All stereoisomers of the compounds disclosed herein and/or pharmaceutically acceptable salts thereof are intended to be included. Unless specifically mentioned otherwise, the reference to one isomer applies to any of the possible isomers. Whenever the isomeric composition is unspecified, all possible isomers are included.

The term “substantially pure” as used herein means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

When compounds disclosed herein contain olefinic double bonds, unless specified otherwise, such double bonds are meant to include both E and Z geometric isomers.

When compounds disclosed herein contain a di-substituted cyclohexyl or cyclobutyl group, substituents found on cyclohexyl or cyclobutyl ring may adopt cis and trans formations. Cis formation means that both substituents are found on the upper side of the 2 substituent placements on the carbon, while trans would mean that they were on opposing sides.

It may be advantageous to separate reaction products from one another and/or from starting materials. The desired product of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

“Diastereomers” refers to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

Some of the compounds disclosed herein may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

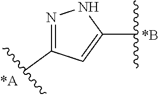 [SMILES: *c1cc(*)[nH]n1]

may undergo tautomerism to form

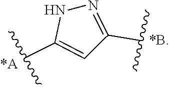 [SMILES: *c1cc(*)[nH]n1]

Wherein *A and *B refer to the position substituents connect to pyrazole.

“Pharmaceutically acceptable salts” refers to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and/or water and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

As defined herein, “a pharmaceutically acceptable salt thereof” includes salts of at least one compound of Formula (I), and salts of the stereoisomers of the compound of Formula (I), such as salts of enantiomers, and/or salts of diastereomers.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as the contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, and rabbit) and most preferably a human.

The term “effective amount” or “therapeutically effective amount” refers to an amount of the active ingredient, such as a compound that, when administered to a subject for treating a disease, or at least one of the clinical symptoms of a disease or disorder, is sufficient to affect such treatment for the disease, disorder, or symptom. The “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer thereof, and/or at least one pharmaceutically acceptable salt thereof disclosed herein effective to “treat” as defined above, a disease or disorder in a subject. In the case of combination therapy, the “therapeutically effective amount” refers to the total amount of the combination objects for the effective treatment of a disease, a disorder or a condition.

The pharmaceutical composition comprising the compound disclosed herein can be administrated via oral, inhalation, rectal, parenteral or topical administration to a subject in need thereof. For oral administration, the pharmaceutical composition may be a regular solid formulation such as tablets, powder, granule, capsules and the like, a liquid formulation such as water or oil suspension or other liquid formulation such as syrup, solution, suspension or the like; for parenteral administration, the pharmaceutical composition may be a solution, water solution, oil suspension concentrate, lyophilized powder or the like. Preferably, the formulation of the pharmaceutical composition is selected from a tablet, coated tablet, capsule, suppository, nasal spray or injection, more preferably tablet or capsule. The pharmaceutical composition can be a single unit administration with an accurate dosage. In addition, the pharmaceutical composition may further comprise additional active ingredients.

All formulations of the pharmaceutical composition disclosed herein can be produced by the conventional methods in the pharmaceutical field. For example, the active ingredient can be mixed with one or more excipients, then to make the desired formulation. The “pharmaceutically acceptable excipient” refers to conventional pharmaceutical carriers suitable for the desired pharmaceutical formulation, for example: a diluent, a vehicle such as water, various organic solvents, etc., a filler such as starch, sucrose, etc. a binder such as cellulose derivatives, alginates, gelatin and polyvinylpyrrolidone (PVP); a wetting agent such as glycerol; a disintegrating agent such as agar, calcium carbonate and sodium bicarbonate; an absorption enhancer such as quaternary ammonium compound; a surfactant such as hexadecanol; an absorption carrier such as Kaolin and soap clay; a lubricant such as talc, calcium stearate, magnesium stearate, polyethylene glycol, etc. In addition, the pharmaceutical composition further comprises other pharmaceutically acceptable excipients such as a decentralized agent, a stabilizer, a thickener, a complexing agent, a buffering agent, a permeation enhancer, a polymer, aromatics, a sweetener, and a dye.

The term “disease” refers to any disease, discomfort, illness, symptoms or indications, and can be interchangeable with the term “disorder” or “condition”.

Throughout this specification and the Aspects which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the Aspects which follow, the term “Cₙ₋ₘ” indicates a range which includes the endpoints, wherein n and M are integers and indicate the number of carbons. Examples include C₁₋₈, C₁₋₆, and the like.

Unless specifically defined elsewhere in this document, all other technical and scientific terms used herein have the meaning commonly understood by one of ordinary skill in the art to which this invention belongs.

### ABBREVIATIONS

DCM dichloromethane
DIEA diisopropylethylamine
DMAP 4-dimethylaminopyridine
DMF Dimethylformamide
DMEM Dulbecco's Modified Eagle Medium
DMSO Dimethyl sulfoxide
FBS Fetal bovine serum
EA Ethyl acetate
EA, EtOAc Ethyl acetate
aq. Aqueous
Eq. equivalent
Et₃N, TEA triethyl amine
EtOH ethanol
HCl Hydrochloric Acid
MeCN acetonitrile
MOH Methanol
NaBH₄ Sodium borohydride
NBS N-Bromosuccinimide
Pd(dppf)Cl₂ [1,1′-Bis(diphenylphosphino)ferrocene]dichloropalladium(II)
PE Petroleum ether
PS Phosphatidylserine
PreP-HPLC Preparative High-Performance Liquid Chromatography
RT or rt room temperature
TFA trifluoroacetic acid
THF tetrahydrofuran
R_{f} Retention Factor
pH Potential Of Hydrogen
t-BuOH tert-butyl alcohol
MtBE Methyl tert-butyl ether
AIBN 2,2′-Azobis(2-methylpropionitrile)
mg Milligrams
mL Milliliters
mmol Millimole
Brettphos Pd G3 Methanesulfonato (2-dicyclohexylphosphino-3,6-dimethoxy-2′,4′,6′-tri-i-propyl-1,1′-biphenyl)(2′-amino-1,1′-biphenyl-2-yl)palladium(II)
m-CPBA m-Chloroperbenzoic Acid
NMR Nuclear Magnetic Resonance
NaHCO₃ Sodium bicarbonate
NH₄Cl Ammonium chloride
NH₄HCO₃ Ammonium bicarbonate
LiBHEt₃ Lithium triethylborohydride
LC-MS (ESI) Liquid Chromatography-Mass Spectrometry (Electrospray Ionization)
K₂CO₃ Potassium carbonate
MHz Megahertz
K₂CO₃ Potassium carbonate
LDA Lithium diisopropylamide
NIS N-Iodosuccinimide
atm standard atmosphere
Na₂SO₄ Sodium sulphate
PhCl Chlorobenzene
PhCl₃ Trichlorobenzene
KOH Potassium hydroxide
N₂ Nitrogen
SOCl₂ Thionyl chloride
NaOH Sodium hydroxide
RuCl₃ Ruthenium(III) chloride
NH₃ Ammonia
IPA Isopropyl alcohol
Hex Hexene
NalO₄ Sodium periodate
CuBr Copper(I) bromide
ATP Adenosine triphosphate
HEPES 4-(2-hydroxyethyl)-1-piperazineethanesulfonic acid
NaN₃ Sodium azide
BSA Bis(trimethylsilyl)acetamide
MgCl₂ Magnesium dichloride
DTT Dithiothreitol
Pd/C Palladium on carbon
BH₃-THF Borane-tetrahydrofuran
sat. Saturated
CO Carbon monoxide

### EXAMPLES

The examples below are intended to be purely exemplary and should not be considered to be limiting in any way. Efforts have been made to ensure accuracy with respect to numbers used (for example, amounts, temperature, etc.), but some experimental errors and deviations should be accounted for. Unless indicated otherwise, temperature is in degrees Centigrade. Reagents were purchased from commercial suppliers such as Sigma-Aldrich, Alfa Aesar, or TCI, and were used without further purification unless indicated otherwise. Unless indicated otherwise, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

¹H NMR spectra were recorded on a Agilent instrument operating at 400 MHz. ¹HNMR spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDCl₃: 7.25 ppm; CD₃OD: 3.31 ppm; D₂O: 4.79 ppm; d₆-DMSO: 2.50 ppm; d₆-acetone: 2.05; (CD₃)₃CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz).

LCMS-1: LC-MS spectrometer (Agilent 1260 Infinity) Detector: MWD (190-400 nm), Mass detector: 6120 SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS-2: LC-MS spectrometer (Agilent 1290 Infinity II) Detector: MWD (190-400 nm), Mass detector: G6125C SQ Mobile phase: A: water with 0.1% Formic acid. B: acetonitrile with 0.1% Formic acid Column: Poroshell 120 EC-C18, 4.6×50 mm, 2.7 pm Gradient method: Flow: 1.2 mL/min Time (min) A (%) B (%)

| Time (min) | A(%) | B(%) |
|---|---|---|
| 0.00 | 90 | 10 |
| 1.5 | 5 | 95 |
| 2.0 | 5 | 95 |
| 2.1 | 90 | 10 |
| 3.0 | 90 | 10 |

LCMS-3: LC-MS spectrometer (Agilent 1260 Infinity II) Detector: MWD (190-400 nm). Mass detector: G6125C SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

UPLC-MS: UPLC Waters Acquity UPLC H-Class, Detector: MWD (190-400 nm), Mass detector: Waters QDA, Mobile phase: A: water with 0.05% TFA, B: acetonitrile with 0.05% TFA, Column: Waters Acquity UPLC BEH C18, 2.1×50 mm, 1.7 μm, Gradient method: Flow: 0.8 mL/min Time (min) A (%) B (%)

| Time (min) | A (%) | B (%) |
|---|---|---|
| 0.00 | 95 | 5 |
| 4.00 | 5 | 95 |
| 4.50 | 5 | 95 |
| 4.51 | 95 | 5 |
| 5.50 | 95 | 5 |

Preparative HPLC was conducted on a column (150×21.2 mm ID, 5 pm. Gemini NXC 18) at a flow rate of 20 ml/min, injection volume 2 ml, at room temperature and UV Detection at 214 nm and 254 nm.

### Example 1: cis-3-(3-((6-fluoro-1,1-dioxido-2,3-dihydrobenzo[b]thiophen-5-yl)amino)-1H-pyrazol-5-yl)cyclopentyl isopropylcarbamate

### Step 1: methyl 1,4-dioxaspiro[4,4]nonane-7-carboxylate

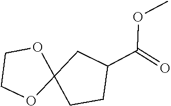

Two parallel reactions were performed. A solution of methyl 3-oxocyclopentanecarboxylate (50.0 g, 351.74 mmol) in toluene (500 mL) was treated with ethylene glycol (43.66 g, 703.47 mmol) and 4-toluenesulfonic acid (6.06 g, 35.17 mmol). The mixture was heated to reflux and stirred for 4 h under nitrogen atmosphere. Each batch was quenched with sat. aq NaHCO₃ separately, then the two bathes were combined and concentrated under reduced pressure. The residue was diluted with EA (2 L) and washed with aq NaHCO₃ (500 mL). The aqueous layer was further extracted with EA (500 mL×2). The combined organic layers were washed with brine (500 mL), dried over anhydrous Na₂SO₄, then filtered and concentrated under reduced pressure. The residue was purified by silica gel column chromatography, eluting with PE/EA (1:1; R_{f}=0.43) to afford the product (34 g, 26%4 yield). ¹H NMR (400 MHz, Chloroform-d) δ=4.00-3.84 (m, 4H), 3.68 (s, 3H), 2.98-2.85 (m, 1H), 2.09 (d, J=8.9 Hz, 2H), 2.06-1.88 (m, 3H), 1.85-1.78 (m, 1H).

### Step 2: 3-oxo-3-(1,4-dioxaspiro[4,4]nonan-7-yl)propanenitrile

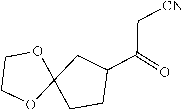 [SMILES: N#CCC(=O)C1CCC2(C1)OCCO2]

Two parallel reactions were performed. To a solution of methyl 1,4-dioxaspiro[4,4]nonane-7-carboxylate (16.0 g, 85.93 mmol) in THF (320 mL), acetonitrile (10.58 g, 257.78 mmol) and sodium hydride (10.31 g, 257.78 mmol, 60%) were added at 0° C. under nitrogen atmosphere. The resulting mixture was heated to reflux and stirred for 4 h. Two batches were combined and poured into sat. aq NH₄Cl (500 mL) at 0° C., and stirred for 30 mins. The layers were separated, and the aqueous layer was extracted with EA (500 mL×3). The combined organic layer was washed with brine (200 mL), dried over anhydrous Na₂SO₄, filtered, and concentrated under vacuum. The residue was purified by silica gel chromatography, eluting with PE/EA (10:1; R_{f}=0.29) to afford the product (30.1 g, 90% yield). ¹H NMR (400 MHz, Chloroform-d) δ=3.96-3.86 (m, 4H), 3.54 (d, J=0.7 Hz, 2H), 3.25-3.10 (m, 1H), 2.11-2.00 (m, 3H), 1.98-1.78 (m, 3H).

### Step 3: 1-(tert-butyl)-3-(1,4-dioxaspiro[4,4]nonan-7-yl)-1H-pyrazol-5-amine

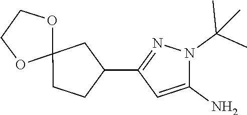 [SMILES: CC(C)(C)n1nc(C2CCC3(C2)OCCO3)cc1N]

To a solution of 3-oxo-3-(1,4-dioxaspiro[4,4]nonan-7-yl)propanenitrile (30.0 g, 153.68 mmol) and tert-butylhydrazine mono hydrochloride (57.45 g, 461.03 mmol) in ethyl alcohol (300 mL) was added triethylamine (46.65 g, 461.03 mmol) at 20° C. The resulting mixture was heated to reflux and stirred for 2 h under nitrogen atmosphere. The reaction mixture was concentrated under reduced pressure, then the residue was purified by silica gel chromatography, eluting with PE/EA (5:1; R_{f}=0.24) to afford the product (27 g, 64% yield). ¹H NMR (400 MHz, DMSO-d₆) δ=5.22 (s, 1H), 4.71 (s, 2H), 3.86-3.76 (m, 4H), 2.89 (tt, J=7.8, 9.9 Hz, 1H), 2.04 (dd, J=8.0, 13.4 Hz, 1H), 1.96-1.82 (m, 2H), 1.78-1.66 (m, 2H), 1.65-1.53 (m, 1H), 1.47 (s, 9H).

### Step 4: benzyl (1-(tert-butyl)-3-(1,4-dioxaspiro[4,4]nonan-7-yl)-1H-pyrazol-5-yl)carbamate

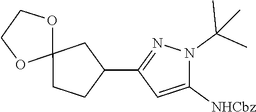 [SMILES: CC(C)(C)n1nc(C2CCC3(C2)OCCO3)cc1NC(=O)OCc1ccccc1]

To a solution of 1-(tert-butyl)-3-(1,4-dioxaspiro[4,4]nonan-7-yl)-1H-pyrazol-5-amine (18 g, 67.83 mmol) in acetone (1 L), benzyl carbonochloridate (23.14 g, 135.67 mmol) was added portion wise at 0° C. The mixture was stirred at room temperature for 2 h, then NaHCO₃ (18.24 g, 217.07 mmol) was added in portions. The mixture was further stirred at this temperature for 26 h. The reaction mixture was filtered and concentrated under reduced pressure to afford the crude product (28.5 g, >99% yield), which was used in the next step without further purification.

### Step 5: benzyl (1-(tert-butyl-3-(3-oxocyclopentyl)-1H-pyrazol-5-yl)carbamate

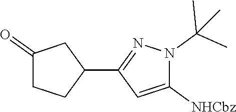 [SMILES: CC(C)(C)n1nc(C2CCC(=O)C2)cc1NC(=O)OCc1ccccc1]

A solution of benzyl (1-(tert-butyl)-3-(1,4-dioxaspiro[4,4]nonan-7-yl)-1H-pyrazol-5-yl)carbamate (27.5 g, 68.84 mmol) in acetone (3 L) and water (300 mL) was treated with 4-toluenesulfonic acid (1.54 g, 8.95 mmol). The mixture was stirred at 60° C. for 4 h under nitrogen atmosphere. The reaction mixture was concentrated under reduced pressure to remove the most of acetone. The aqueous residue was extracted with DCM (500 mL×3). The combined organic phase was washed with brine (500 mL), dried with anhydrous Na₂SO₄, then filtered and concentrated under vacuum. The residue was purified by silica gel chromatography, eluting with PE/EA (10:1) to afford the product (23 g, 87% yield). ¹H NMR (400 MHz, DMSO-d₄) δ=9.12 (br s, 1H), 7.44-7.28 (m, 5H), 6.03 (s, 1H), 5.12 (s, 2H), 3.35-3.32 (m, 1H), 2.48-2.41 (m, 1H), 2.33-2.18 (m, 4H), 1.97-1.87 (m, 1H), 1.48 (s, 9H).

### Step 6: cis-benzyl (1-tert-butyl)-3-(3-hydroxycyclopentyl)-1H-pyrazol-5-yl)carbamate

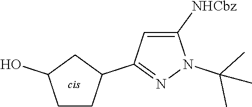 [SMILES: CC(C)(C)n1nc(C2CCC(O)C2)cc1NC(=O)OCc1ccccc1]

A solution of benzyl (1-(tert-butyl)-3-(3-oxocyclopentyl)-1H-pyrazol-5-yl)carbamate (19 g, 53.46 mmol) in THF (200 mL) was cooled to −65° C. A solution of LiBHEt₃ (1 M, 106.9 mL) was added dropwise and the resulting mixture was stirred at −65° C. for 1.5 h under nitrogen atmosphere. The reaction mixture was quenched with sat. aq. NaHCO₃ (50 mL). Water (200 mL) was added, and the mixture was extracted with EA (100 mL×3). The combined organic phase was washed with brine (200 mL), dried over anhydrous Na₂SO₄, then filtered and concentrated under vacuum. The residue was purified by prep-HPLC (column; mobile phase: [water (NH₄HCO₃)-acetone]; B %: 40/6-65%, 20 min). Benzyl (1-(tert-butyl)-3-((1S,3R)-3-hydroxycyclopentyl)-1H-pyrazol-5-yl)carbamate (11 g, 57% yield) was obtained. ¹H NMR (400 MHz, DMSO-d₆) δ=9.06 (br s, 1H), 7.57-7.12 (m, 51), 5.92 (s, 1H), 5.75 (s, 1H), 5.12 (s, 2H), 4.57 (d, J=4.4 Hz, 1H), 4.22-4.06 (m, 1H), 2.89 (q, J=8.6 Hz, 1H), 2.25-2.13 (m, 1H), 1.89-1.80 (m, 1H), 1.76-1.66 (m, 2H), 1.60-1.55 (m, 1H), 1.54-1.41 (m, 9H).

### Step 7: cis-benzyl (1-(tert-butyl)-3-(3-(((4-nitrophenoxy)carbonyl)oxy)cyclopentyl)-1H-pyrazol-5-yl)carbamate

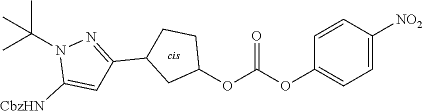 [SMILES: CC(C)(C)n1nc(C2CCC(OC(=O)Oc3ccc([N+](=O)[O-])cc3)C2)cc1NC(=O)OCc1ccccc1]

To a solution of cis-benzyl (1-(tert-butyl)-3-(3-hydroxycyclopentyl)-1H-pyrazol-5-yl)carbamate (10 g, 28.01 mmol) in DCM (100 mL) was added pyridine (11.1 g, 140 mmol). DMAP (169 mg, 1.4 mmol), and 4-nitrophenyl carbonochloridate (6.7 g, 33.5 mmol), successively. The resulting mixture was stirred at room temperature for 16 h. The reaction mixture was concentrated under reduced pressure, and the residue was purified by silica gel chromatography, eluting with PE/EA (3:1) to afford the product (11.7 g, 80% yield). LC-MS (ESI): m/z [M+H]⁺=523.2.

### Step 8: cis-benzyl (5-(3-(((4-nitrophenoxy)carbonyl)oxy)cyclopentyl)-1H-pyrazol-3-yl)carbamate

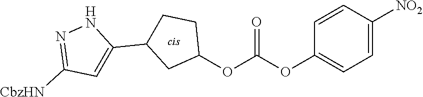 [SMILES: O=C(Nc1cc(C2CCC(OC(=O)Oc3ccc([N+](=O)[O-])cc3)C2)[nH]n1)OCc1ccccc1]

To a round-bottom flask charged with cis-benzyl (1-(tert-butyl)-3-(3-(((4-nitrophenoxy)carbonyl)oxy)cyclopentyl)-1H-pyrazol-5-yl)carbamate (30 g, 57.5 mmol) was added 100 mL formic acid. The resulting mixture was stirred at 75° C. for over night. The solvent was removed under vacuum to yield 26 g of crude product, which was directly used for the next step without further purification. LC-MS (ESI): m/z [M+H]⁺=467.2.

### Step 9: cis-3-(3-(((benzyloxy)carbonyl)amino)-1H-pyrazol-5-yl)cyclopentyl isopropylcarbamate

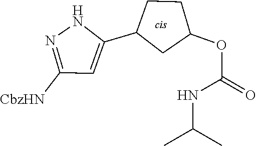 [SMILES: CC(C)NC(=O)OC1CCC(c2cc(NC(=O)OCc3ccccc3)n[nH]2)C1]

To a solution of cis-benzyl (5-(3-(((4-nitrophenoxy)carbonyl)oxy)cyclopentyl)-1H-pyrazol-3-yl)carbamate (7 g, 15 mmol) in 100 mL THF was added propan-2-amine (2.6 g, 45 mmol). The resulting mixture was stirred at room temperature for 1 h. The resulting mixture was concentrated under reduced pressure, and the residue was purified by silica gel chromatography (PE/EA=2:1) to afford the product (4.6 g, 80% yield). LC-MS (ESI): m/z [M+H]⁺=387.3.

### Step 10: cis-3-(3-amino-1H-pyrazol-5-yl)cyclopentyl isopropylcarbamate

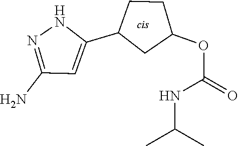 [SMILES: CC(C)NC(=O)OC1CCC(c2cc(N)n[nH]2)C1]

To a solution of cis-3-(3-(((benzyloxy)carbonyl)amino)-1H-pyrazol-5-yl)cyclopentyl isopropylcarbamate (4.6 g, 12 mmol) in THF (100 mL) was added Pd/C (10%, wet, 3 g). The suspension was degassed and purged with hydrogen 3 times. The resulting mixture was stirred at room temperature under a hydrogen balloon for 3 h. The mixture was filtered, and the filter cake was washed with EA (50 mL×3). The filtrate was concentrated under reduced pressure to afford the product (2.5 g, 82% yield). LC-MS (EST): m/z [M+H]⁺=253.4.

### Step 11: (4-bromo-3-fluorophenyl)(2,2-diethoxyethyl)sulfane

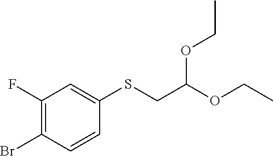 [SMILES: CCOC(CSc1ccc(Br)c(F)c1)OCC]

To a solution of 4-bromo-3-fluorobenzenethiol (2.0 g, 9.7 mmol) and 2-bromo-1,1-diethoxyethane (2.87 g, 14.56 mmol) in DMF (20 mL) was added K₂CO₃ (2.0 g, 14.56 mmol), the reaction mixture was stirred for 7 hours at 135° C. under nitrogen atmosphere. The resulting mixture was cooled to room temperature, diluted with water (100 mL), extracted with EtOAc (3-100 mL), the combined organic phase was washed with water, then brine, dried over Na₂SO₄, filtered, concentrated under reduce pressure, dried over in vacuo, to afford the crude product. (3.0 g crude). ¹H NMR (500 MHz, DMSO-d₆) δ 7.59 (dd, J=8.3, 7.7, 1H), 7.42 (dd, J=9.9, 2.2, 1H), 7.14 (dd, J=8.4, 1.9, 1H), 4.63 (t, J=5.4, 1H), 3.60 (m, 2H), 3.49 (m, 2H), 3.20 (d, J=5.5, 2H), 1.09 (t, J=7.0, 6H).

### Step 12: 5-bromo-6-fluorobenzo[b]thiophene

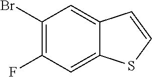 [SMILES: Fc1cc2sccc2cc1Br]

To a solution of (4-bromo-3-fluorophenyl)(2,2-diethoxyethyl)sulfane (2.0 g, 6.21 mmol) in PhCl (60 mL) was added polyphosphoric acid (3.6 g), the reaction mixture was stirred for 16 hours at 130° C. The resulting mixture was cooled to room temperature, diluted with water (100 mL), extracted with EtOAc (3*100 mL), the combined organic phase was washed with saturated Na₂CO₃ aqueous, then brine, dried over Na₂SO₄, filtered, concentrated under reduce pressure, purified by silica gel column chromatography, eluting with PE to afford the product (1.2 g crude, 5-bromo-4-fluorobenzo[b]thiophene:5-bromo-6-fluorobenzo[b]thiophene=1:4).

### Step 13: 5-bromo-6-fluorobenzo[b]thiophene 1,1-dioxide

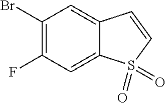 [SMILES: O=S1(=O)C=Cc2cc(Br)c(F)cc21]

A mixture of 5-bromo-4-fluorobenzo[b]thiophene and 5-bromo-6-fluorobenzo[b]thiophene (956 mg crude, 4.156 mmol, 1:4) was dissolved in DCM (60 mL), m-CPBA (2.86 g, 12.47 mmol) was added one portion, the reaction mixture was stirred for 16 hours at room temperature. The resulting mixture was washed with saturated Na₂CO₃ aqueous, the brine, dried over Na₂SO₄, filtered, concentrated under reduce pressure, purified by silica gel column chromatography, eluting with PE/EA (1:1) to afford the product (420 mg, 30% for 2 steps). LC-MS (ESI): m/z [M+H]⁺=263.1.

### Step 14: 5-bromo-6-fluoro-2,3-dihydrobenzo[b]thiophene 1,1-dioxide

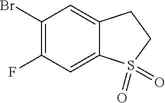 [SMILES: O=S1(=O)CCc2cc(Br)c(F)cc21]

To a solution of 5-bromo-6-fluorobenzo[b]thiophene 1,1-dioxide (420 mg, 1.7 mmol) in EtOH (10 mL) was added NaBH₄ (129 mg, 3.4 mmol), the reaction mixture was stirred for 6 hours at room temperature. The resulting mixture was concentrated, purified by prep-TLC (PE/EA=4:1) to afford the product (251 mg, 56%). LC-MS (ESI): m/z [M+H]⁺=265.0.

### Step 15: cis-3-(3-(6-fluoro-1,1-dioxido-2,3-dihydrobenzo[b]thiophen-5-yl)amino)-1H-pyrazol-5-yl)cyclopentyl isopropylcarbamate

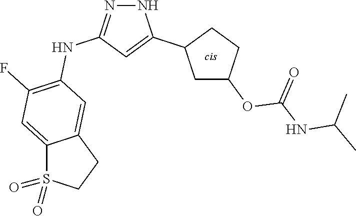 [SMILES: CC(C)NC(=O)OC1CCC(c2cc(Nc3cc4c(cc3F)S(=O)(=O)CC4)n[nH]2)C1]

To a solution of 5-bromo-6-fluoro-2,3-dihydrobenzo[b]thiophene 1,1-dioxide (100 mg, 0.38 mmol) and cis-3-(3-amino-1H-pyrazol-5-yl)cyclopentyl isopropylcarbamate (100 mg, 0.38 mmol) in t-BuOH (10 mL) was added BrettPhos Pd G3 (34 mg, 0.038 mmol) and K₂CO₃ (160 mg, 1.4 mmol), the reaction mixture was stirred for 16 hours at 110° C. under nitrogen atmosphere. The resulting mixture was cooled to room temperature, filtered, the filtrate was concentrated under reduce pressure, purified by silica gel column chromatography, eluting with PE/EA (1:4) to afford the product (136 mg, 82%). ¹H NMR (500 MHz, DMSO-d₆) δ 11.99 (s, 1H), 8.74 (s, 1H), 8.14 (s, 1H), 7.54 (d, J=10.1 Hz, 1H), 6.95 (d, J=7.3 Hz, 1H), 5.85 (s, 1H), 5.00 (s, 1H), 3.64-3.51 (m, 3H), 3.25 (t, J=6.7 Hz, 2H), 3.11-3.01 (m, 1H), 2.45 (d, J=7.3 Hz, OH), 2.09-1.98 (m, 1H), 1.96-1.84 (m, 1H), 1.78-1.65 (m, 2H), 1.63-1.53 (m, 1H), 1.03 (d, J=6.1 Hz, 6H). LC-MS (ESI): m/z [M+H]⁺=437.2.

### Example 2: cis-3-(3-((4-fluoro-1,1-dioxido-2,3-dihydrobenzo[b]thiophen-5-yl)amino)-1H-pyrazol-5-yl)cyclopentyl isopropylcarbamate

### Step 1: (4-bromo-3-fluorophenyl)(2,2-diethoxyethyl)sulfane

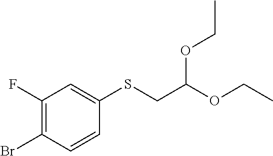 [SMILES: CCOC(CSc1ccc(Br)c(F)c1)OCC]

To a solution of 4-bromo-3-fluorobenzenethiol (2.0 g, 9.7 mmol) and 2-bromo-1,1-diethoxyethane (2.87 g, 14.56 mmol) in DMF (20 mL) was added K₂CO₃ (2.0 g, 14.56 mmol), the reaction mixture was stirred for 7 hours at 135° C. under nitrogen atmosphere. The resulting mixture was cooled to room temperature, diluted with water (100 mL), extracted with EtOAc (3-100 mL), the combined organic phase was washed with water, then brine, dried over Na₂SO₄, filtered, concentrated under reduce pressure, and dried over in vacuo to afford the crude product. (3.0 g). ¹H NMR (500 MHz, DMSO-d₆) δ=7.59 (dd, J=8.3, 7.7, 1H), 7.42 (dd, J=9.9, 2.2, 1H), 7.14 (dd, J=8.4, 1.9, 1H), 4.63 (t, J=5.4, 1H), 3.60 (m, 2H), 3.49 (m, 2H), 3.20 (d, J=5.5, 2H), 1.09 (t, J=7.0, 6H).

### Step 2: 5-bromo-4-fluorobenzo[b]thiophene

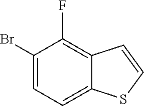 [SMILES: Fc1c(Br)ccc2sccc12]

To a solution of (4-bromo-3-fluorophenyl)(2,2-diethoxyethyl)sulfane (2.0 g, 6.21 mmol) in chlorobenzene (60 mL) was added polyphosphoric acid (3.6 g), the reaction mixture was stirred for 16 hours at 130° C. The resulting mixture was cooled to room temperature, diluted with water (100 mL), extracted with EtOAc (3×100 mL), the combined organic phase was washed with saturated Na₂CO₃ aqueous, then brine, dried over Na₂SO₄, filtered, concentrated under reduce pressure, purified by silica gel column chromatography, eluting with PE to afford the product (1.2 g crude, 5-bromo-4-fluorobenzo[b]thiophene:5-bromo-6-fluorobenzo[b]thiophene=1:4).

### Step 3: 5-bromo-4-fluorobenzo[b]thiophene 1,1-dioxide

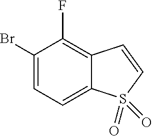 [SMILES: O=S1(=O)C=Cc2c1ccc(Br)c2F]

To a solution of 5-bromo-4-fluorobenzo[b]thiophene and 5-bromo-6-fluorobenzo[b]thiophene (956 mg crude, 4.156 mmol, isomers 1:4) was dissolved in DCM (60 mL), m-CPBA (2.86 g, 12.47 mmol) was added one portion, the reaction mixture was stirred for 16 hours at room temperature. The resulting mixture was washed with saturated Na₂CO₃ aqueous, the brine, dried over Na₂SO₄, filtered, concentrated under reduce pressure, purified by silica gel column chromatography, eluting with PE/EA (1:1) to afford the product (110 mg, 7% for 2 steps). LC-MS (ESI): m/z [M+H]⁺=263.1;

### Step 4: 5-bromo-4-fluoro-2,3-dihydrobenzo[b]thiophene 1,1-dioxide

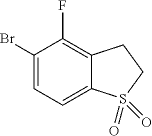 [SMILES: O=S1(=O)CCc2c1ccc(Br)c2F]

To a solution of 5-bromo-4-fluorobenzo[b]thiophene 1,1-dioxide (110 mg, 0.42 mmol) in EtOH (5 mL) was added NaBH₄ (48 mg, 1.26 mmol), the reaction mixture was stirred for 6 hours at room temperature. The resulting mixture was concentrated, purified by prep-TLC (PE/EA=4:1) to afford the product (100 mg, 91%). ¹H NMR (500 MHz, DMSO-d₆) δ=7.90 (dd, J=8.1, 6.1, 1H), 7.59 (d, J=8.2, 1H), 3.71-3.65 (m, 2H), 3.40 (t, J=6.8, 2H). LC-MS (ESI): m/z [M+H]⁺=265.0.

### Step 5: cis-3-(3-((4-fluoro-1,1-dioxido-2,3-dihydrobenzo[b]thiophen-5-yl)amino)-1H-pyrazol-5-yl)cyclopentyl isopropylcarbamate

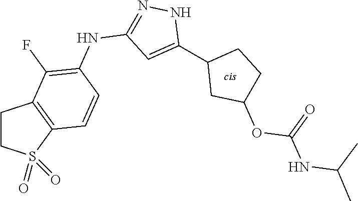 [SMILES: CC(C)NC(=O)OC1CCC(c2cc(Nc3ccc4c(c3F)CCS4(=O)=O)n[nH]2)C1]

To a solution of 5-bromo-4-fluoro-2,3-dihydrobenzo[b]thiophene 1,1-dioxide (100 mg, 0.38 mmol) and 3-(3-amino-1H-pyrazol-5-yl)cyclopentyl isopropylcarbamate (100 mg, 0.38 mmol) in t-BuOH (10 mL) was added BrettPhos Pd G3 (34 mg, 0.038 mmol) and K₂CO₃ (160 mg, 1.4 mmol), the reaction mixture was stirred for 16 hours at 110° C. under nitrogen atmosphere. The resulting mixture was cooled to room temperature, filtered, the filtrate was concentrated under reduce pressure, purified by silica gel column chromatography, eluting with PE/E A (4:1-1:10) to afford the product (150 mg, 90%) in a racemic form, which was further separated by Chiral Prep-HPLC to give:

Enantiomer 1 (Example 2a, 100% ee); Retention time: 5.27 min. ¹H NMR (500 MHz, DMSO-d₆) δ 11.98 (s, 1H), 8.70 (s, 1H), 8.23 (t, J=8, 1, 1H), 7.41 (d, J=8.6, 1H), 6.94 (d, J=7.4, 1H), 5.83 (d, J=2.0, 1H), 5.00 (m, 1H), 3.56 (m, 3H), 3.33 (m, 1H), 3.30 (m, 1H), 3.11-3.01 (m, 1H), 2.48-2.42 (m, 1H), 2.06-1.97 (m, 1H), 1.96-1.84 (m, 1H), 1.78-1.64 (m, 21f), 1.59 (m, 1H), 1.07-0.96 (m, 6H). LC-MS (ESI): m/z [M+H]⁺=437.33.

Enantiomer 2 (Example 2b, 100% ee): Retention time: 9.341 min. ¹H NMR (500 MHz, DMSO-d₆) δ 11.98 (s, 1H), 8.70 (s, 1H), 8.23 (t, J=8.1, 1H), 7.41 (d, J=8.6, 1H), 6.94 (d, J=7.4, 1H), 5.83 (d, J=2.0, 1H), 5.00 (m, 1H), 3.56 (m, 3H), 3.33 (m, 1H), 3.30 (m, 1H), 3.11-3.01 (m, 1H), 2.48-2.42 (m, 1H), 2.06-1.97 (m, 1H), 1.96-1.84 (m, 1H), 1.78-1.64 (m, 2H), 1.59 (m, 1H), 1.07-0.96 (m, 6H). LC-MS (ESI): m/z [M+H]⁺=437.33.

Chiral analytical method: Column: CHIRALPAK IE 4.6×250 mm 5 μm; Mobile phase: A for MtBE (0.1% 2M NH₃ MeOH) and B for MeOH:DCM=50:50 (v/v); Gradient: Mobile Phase A:Mobile Phase B=10:90 (v/v); HPLC Equipment: HPLC-Agilent; Column temperature: 35° C.

Chiral Prep-HPLC Condition: CHIRALPAK IE 20×250 mm 5 μm; Mobile phase: A for MtBE and B for MeOH:DCM=50:50 (0.2% 2M NH₃ MeOH); Gradient: Mobile Phase A:Mobile Phase B=10:90 (v/v); Flow Rate: 18 mL/min, Wavelength: UV 280 nm and 300 nm, Prep-HPLC Equipment: Prep-HPLC-Gilson; Column temperature: 25° C.

### Example 3: cis-3-(3-((7-fluoro-1,1-dioxido-2,3-dihydrobenzo[b]thiophen-6-yl)amino)-1H-pyrazol-5-yl)cyclopentyl isopropylcarbamate

### Step 1: S-(3-bromo-2-fluorophenyl) ethanethioate

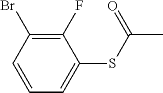

A mixture of 1-bromo-2-fluoro-3-iodobenzene (6 g, 20 mmol), Potassium thioacetate (4.57 g, 40 mmol), o-Phenanthroline (7.2 g, 40 mmol) and CuI (0.6 g, 10% wt), in PhMe (100 mL) was stirred for 3 h at 100° C. under N₂ atmosphere. The resulting mixture was concentrated under reduced pressure. The residue was purified by silica gel column chromatography, eluting with PE/EtOAc (5:1) to afford the product (3 g, 60.5%). LC-MS (ESI): m/z [M+H]⁺=249.5.

### Step 2: 3-bromo-2-fluorobenzenethiol

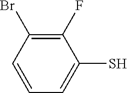

A mixture of S-(3-bromo-2-fluorophenyl) ethanethioate (3 g, 12 mmol) and KOH (0.81 g, 14.4 mmol) in THF/MeOH (1:1, 50 mL) was stirred for 16 h at 20° C. The mixture acided to pH=6 with saturated HCl (aq. 10%) and extracted with EA (100 mL×2). The organic phase was concentrated under reduced pressure. The mixture was concentrated under reduced pressure. The residue was purified by silica gel column chromatography, eluting with PE/EtOAc (1:1) to afford the product (1 g, 40.2%), LC-MS (EST): m/z [M+H]⁺=207.4.

### Step 3: (3-bromo-2-fluorophenyl)(2,2-diethoxyethyl)sulfane

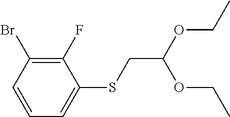 [SMILES: CCOC(CSc1cccc(Br)c1F)OCC]

A solution of 3-bromo-2-fluorobenzenethiol (1 g, 4.8 mmol), 2-bromo-1,1-diethoxyethane (1.4 g, 7.2 mmol) and K₂CO₃ (2 g, 14.4 mmol) in DMF (20 mL) was stirred at 135° C. for 16 h. The solution was concentrated under reduced pressure. The residue was purified by silica gel column chromatography, eluting with PE/EtOAc (4:1) to afford the product (1.3 g, 84%). [M+H]⁺=323.2.

### Step 4: 6-bromo-7-fluorobenzo[b]thiophene

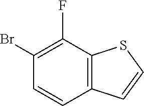 [SMILES: Fc1c(Br)ccc2ccsc12]

A solution of (3-bromo-2-fluorophenyl)(2,2-diethoxyethyl)sulfane (1.3 g, 4 mmol) and polyphosphoric acid (10 mL) in PhCl₃ (50 mL) was stirred at 130° C. for 16 h. The solution was concentrated under reduced pressure. The residue was purified by silica gel column chromatography, eluting with PE/EtOAc (3:1) to afford the product (0.5 g, 52.5%). LC-MS (ESI): m/z [M+H]⁺=231.4.

### Step 5: 6-bromo-7-fluorobenzo[b]thiophene 1,1-dioxide

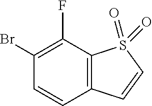 [SMILES: O=S1(=O)C=Cc2ccc(Br)c(F)c21]

A solution of 6-bromo-7-fluorobenzo[b]thiophene (0.5 g, 2.1 mmol) and m-CPBA (1.1 g, 6.3 mmol) in DCM (20 mL) was stirred at 20° C. for 16 h. The solution was concentrated under reduced pressure. The residue was purified by silica gel column chromatography, eluting with PE/EtOAc (1:1) to afford the product (0.5 g, 90% 1.9 mmol), LC-MS (ESI): m/z [M+H]⁺=263.3.

### Step 6: 6-bromo-7-fluoro-2,3-dihydrobenzo[b]thiophene 1,1-dioxide

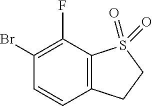 [SMILES: O=S1(=O)CCc2ccc(Br)c(F)c21]

A solution of 6-bromo-7-fluorobenzo[b]thiophene 1,1-dioxide (0.5 g, 1.9 mmol) and NaBH₄ (145 g, 3.8 mmol) in EtOH (10 mL) was stirred at 20° C. for 16 h. The solution was concentrated under reduced pressure. The residue was purified by silica gel column chromatography, eluting with PE/EtOAc (1:1) to afford the product (0.4 g, 79%), LC-MS (ESI): m/z [M+H]⁺=265.4.

### Step 7: cis-3-(3-4(7-fluoro-1,1-dioxido-2,3-dihydrobenzo[b]thiophen-6-yl)amino)-1H-pyrazol-5-yl)cyclopentyl isopropylcarbamate

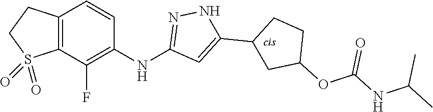 [SMILES: CC(C)NC(=O)OC1CCC(c2cc(Nc3ccc4c(c3F)S(=O)(=O)CC4)n[nH]2)C1]

A mixture of 6-bromo-7-fluoro-2,3-dihydrobenzo[b]thiophene 1,1-dioxide (100 mg, 0.37 mmol), cis-3-(3-amino-1H-pyrazol-5-yl)cyclopentyl isopropylcarbamate (93 mg, 0.37 mmol), Brettephos Pd G3 (36 mg, 0.04 mmol) and K₂CO₃ (153 mg, 1.11 mmol) in t-BuOH (15 mL) was stirred for 16 h at 110° C. in the sealed tube under N₂ atmosphere. The solution was concentrated under reduced pressure. The residue was purified by silica gel column chromatography, eluting with PE/EtOAc (4:1-1:10) to afford the crude product (0.1 g). Then the crude product was further purified by prep HPLC (Waters XSelect C18: RD-CO-094 column, eluting with a gradient of acetonitrile/water containing 0.1% FA, 20%-55%) to afford the product in a racemic form was further separated by Chiral Prep-HPLC to give:

Enantiomer 1 (Example 3a, 100% ee); Retention time: 7.08 min. ¹H NMR (500 MHz, DMSO-d₆) δ 11.86 (s, 1H), 8.43 (s, 1H), 8.35 (s, 1H), 7.16 (d, J=8.4 Hz, 1H), 6.93 (s, 1H), 5.75 (s, 1H), 5.05-4.9 (m, 1H), 3.70-3.50 (m, 3H), 3.28-3.222 (m, 2H), 3.10-2.95 (m, 1H), 2.47-2.41 (m, 1H), 2.08-1.98 (m, 1H), 1.95-1.85 (m, 1H), 1.76-1.65 (m, 2H), 1.63-1.55 (m, 1H), 1.10-0.98 (m, 6H). LC-MS (ESI): m/z [M+H]⁺=437.28.

Enantiomer 2 (Example 3b, 97.54% ee); Retention time: 8.86 min. ¹H NMR (500 MHz, DMSO-d₆) δ 11.86 (s, 1H), 8.43 (s, 1H), 8.35 (s, 1H), 7.16 (d, J=8.4 Hz, 1H), 6.93 (s, 1H), 5.75 (s, 1H), 5.05-4.9 (m, 1H), 3.70-3.50 (m, 3H), 3.28-3.222 (m, 2H), 3.10-2.95 (m, 1H), 2.47-2.41 (m, 1H), 2.08-1.98 (m, 1H), 1.95-1.85 (m, 1H), 1.76-1.65 (m, 2H), 1.63-1.55 (m, 1H), 1.10-0.98 (m, 6H). LC-MS (ESI): m/z [M+H]⁺=437.28.

Chiral analytical method: Column: CHIRALPAK IE 4.6 mm×250 mm, 5 μm; Mobile phase: A for Hexane and B for EtOH (0.5% NH₃ MeOH); Gradient: Mobile Phase A:Mobile Phase B=20:80 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for Hexane and B for EtOH (0.5% NH₃ MeOH): Gradient: Mobile Phase A:Mobile Phase B=20:80 (v/v); Flow Rate: 18 mL/min, Wave Length: UV 200 nm and 270 nm, Prep-HPLC Equipment: Prep-HPLC-Gilson. Back pressure: 100 bar: Column temperature: 25° C.

### Example 4: cis-3-(3-((7-fluoro-1,1-dioxido-2,3-dihydrobenzo[b]thiophen-6-yl)amino)-1H-pyrazol-5-yl)cyclopentyl propylcarbamate

### Step 1: cis-3-(3-amino-1H-pyrazol-5-yl)cyclopentyl propylcarbamate

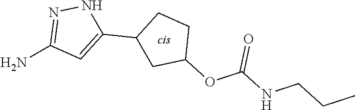 [SMILES: CCCNC(=O)OC1CCC(c2cc(N)n[nH]2)C1]

The tided compound was synthesized in the procedures similar to Example 1. LC-MS (ESI): m/z [M+H]⁺=253.3

### Step 2: cis-3-(34(7-fluoro-1,1-dioxido-2,3-dihydrobenzo[b]thiophen-6-yl)amino)-1H-pyrazol-5-yl)cyclopentyl propylcarbamate

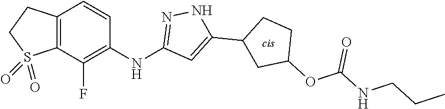 [SMILES: CCCNC(=O)OC1CCC(c2cc(Nc3ccc4c(c3F)S(=O)(=O)CC4)n[nH]2)C1]

The tided compound was synthesized in the procedures similar to Example 3. ¹H NMR (500 MHz, DMSO-d₆) δ 11.96 (brs, 1H), 8.42 (s, 1H), 8.33 (t, J=7.9 Hz, 1H), 7.16 (d, J=8.4 Hz, 1H), 7.04 (s, 1H), 5.75 (s, 1H), 5.10-4.95 (m, 1H), 3.64 (t, J=6.8 Hz, 2H), 3.25 (t, J=6.8 Hz, 2H), 3.12-2.99 (m, 1H), 2.98-2.85 (m, 2H), 2.48-2.42 (m, 1H), 2.05-1.98 (m, 1H), 1.96-1.85 (m, 1H), 1.80-1.65 (m, 2H), 1.64-1.55 (m, 1H), 1.45-1.32 (m, 2H), 0.82 (t, J=7.3 Hz, 3H). LC-MS (ESI): m/z [M+H]⁺=437.32.

### Example 5: cis-3-(3-((7-fluoro-1,1-dioxido-2,3-dihydrobenzo[b]thiophen-6-yl)amino)-1H-pyrazol-5-yl)cyclopentyl ((S)-sec-butyl)carbamate

### Step 1: cis-3-(3-amino-1H-pyrazol-5-yl)cyclopentyl ((S)-sec-butyl)carbamate

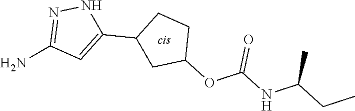 [SMILES: CC[C@H](C)NC(=O)OC1CCC(c2cc(N)n[nH]2)C1]

The tided compound was synthesized in the procedures similar to Example 1. LC-MS (ESI): m/z [M+H]⁺=267.2.

### Step 2: cis-3-(34(7-fluoro-1,1-dioxido-2,3-dihydrobenzo[b]thiophen-6-yl)amino)-1H-pyrazol-5-yl)cyclopentyl ((S)-sec-butyl)carbamate

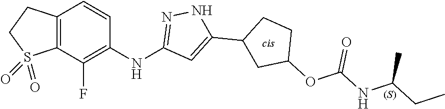

The tided compound was synthesized in the procedures similar to Example 3. ¹H NMR (500 MHz, DMSO-d₆) δ 11.87 (s, 1H), 8.42 (s, 1H), 8.33 (s, 1H), 7.16 (d, J=8.5 Hz, 1H), 6.88 (d, J=8.0 Hz, 1H), 5.75 (s, 1H), 5.05-4.95 (m, 1H), 3.64 (t, J=6.9 Hz, 2H), 3.45-3.36 (m, 1H), 3.25 (t, J=6.9 Hz, 2H), 3.11-2.98 (m, 1H), 2.48-2.41 (m, 1H), 2.06-1.98 (m, 1H), 1.95-1.85 (m, 1H), 1.78-1.66 (m, 2H), 1.64-1.54 (m, 1H), 1.42-1.27 (m, 2H), 1.05-0.92 (m, 3H), 0.80 (q, J=7.0 Hz, 3H). LC-MS (ESI): m/z [M+H]⁺=451.33.

### Example 6: cis-3-(3-((7-fluoro-1,1-dioxido-2,3-dihydrobenzo[b]thiophen-6-yl)amino)-1H-pyrazol-5-yl)cyclopentyl (1-methylcyclopropyl)carbamate

### Step 1: cis-3-(3-amino-1H-pyrazol-5-yl)cyclopentyl (1-methylcyclopropyl)carbamate

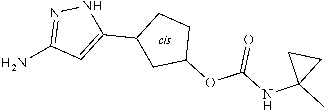 [SMILES: CC1(NC(=O)OC2CCC(c3cc(N)n[nH]3)C2)CC1]

The titled compound was synthesized in the procedures similar to Example 1. LC-MS (ESI): m/z [M+H]⁺=265.2.

### Step 2: cis-3-(3-((7-fluoro-1,1-dioxido-2,3-dihydrobenzo[b]thiophen-6-yl)amino)-1H-pyrazol-5-yl)cyclopentyl (1-methylcycloproyl)carbamate

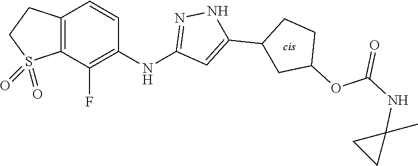 [SMILES: CC1(NC(=O)OC2CCC(c3cc(Nc4ccc5c(c4F)S(=O)(=O)CC5)n[nH]3)C2)CC1]

The titled compound was synthesized in the procedures similar to Example 3. ¹H NMR (500 MHz, DMSO-d₆) δ 11.86 (s, 1H), 8.50-8.40 (m, 1H), 8.35 (t., J=8.1 Hz, 1H), 7.34 (s, 1H), 7.16 (d, J=8.5 Hz, 1H), 5.74 (s, 1H), 5.05-4.95 (m, 1H), 3.64 (t, J=6.9 Hz, 2H), 3.25 (t, J=6.9 Hz, 2H), 3.10-2.98 (m, 1H), 2.48-2.42 (m, 1H), 2.05-1.95 (m, 1H), 1.94-1.85 (m, 1H), 1.78-1.62 (m, 2H), 1.60-1.50 (m, 1H), 1.23 (s, 4H), 0.63-0.55 (m, 2H), 0.53-0.45 (m, 2H). LC-MS (ESI): m/z [M+H]⁺=449.35.

### Example 7: cis-3-(3-((2,2-dioxido-1,3-dihydrobenzo[c]thiophen-5-yl)amino)-1H-pyrazol-5-yl)cyclopentyl isopropylcarbamate

### Step 1: cis-3-(3-((2,2-dioxido-1,3-dihydrobenzo[c]thiophen-5-yl)amino)-1H-pyrazol-5-yl)cyclopentyl isopropylcarbamate

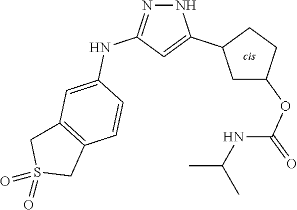 [SMILES: CC(C)NC(=O)OC1CCC(c2cc(Nc3ccc4c(c3)CS(=O)(=O)C4)n[nH]2)C1]

A mixture of 5-bromo-1,3-dihydrobenzo[c]thiophene 2,2-dioxide (50 mg, 0.20 mmol), 3-(3-amino-1H-pyrazol-5-yl)cyclopentyl isopropylcarbamate (50.4 mg, 0.20 mmol), Brettphos Pd G3 (18.2 mg, 0.02 mmol). K₂CO₃ (82.8 mg, 0.60 mmol) in t-BuOH (10 mL) was stirred at 110° C. for 16 h under N₂ atmosphere. The mixture was concentrated under reduced pressure. The residue was purified by silica gel column chromatography. The residue was purified by prep HPLC (Waters XSelect C18: RD-CO-094 column, eluting with a gradient of acetonitrile/water containing 0.1% FA, 20%-40%) to afford the product (25 mg, 31.2%). ¹H NMR (500 MHz, DMSO-d₆) δ 11.76 (s, 1H), 8.47 (s, 1H), 7.39 (s, 1H), 7.23-7.19 (m, 1H), 7.13 (d, J=8.4 Hz, 1H), 6.95 (d, J=7.2 Hz, 1H), 5.63 (s, 1H), 4.98 (s, 1H), 4.40 (s, 2H), 4.32 (s, 2H), 3.63-3.51 (m, 1H), 3.11-2.98 (m, 1H), 2.48-2.38 (m, 1H), 2.05-1.97 (m, 1H), 1.95-1.81 (m, 1H), 1.78-1.64 (m, 2H), 1.63-1.53 (m, 1H), 1.03 (d, J=6.4 Hz, 6H). LC-MS (ESI): m/z [M+H]⁺=419.2.

### Example 8: cis-3-(3-((4-fluoro-2,2-dioxido-1,3-dihydrobenzo[c]thiophen-5-yl)amino)-1H-pyrazol-5-yl)cyclopentyl isopropylcarbamate

### Step 1: 4-bromo-3-fluorophthalic acid

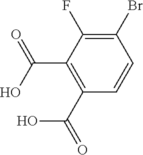 [SMILES: O=C(O)c1ccc(Br)c(F)c1C(=O)O]

To a solution of 4-bromo-3-fluorobenzoic acid (30 g, 137.6 mmol) in THF (600 mL) were added LDA (172 mL, 344 mmol) at −78° C. under nitrogen atmosphere for 1 h. The dry ice (45 g) was added in it at −78° C. for 1 h. The reaction was quenched with aq. NH₄Cl (200 mL). The resulting solution was concentrated under vacuum. The resulting mixture was quenched with MeOH (500 mL). The resulting mixture was stirred at rt for 15 min. The resulting mixture was filtered, the filter cake was washed with MeOH. The filtrate was concentrated under reduced pressure. The residue was the crude product (30 g, 83%). LCMS (ESI) m/z [M−H]⁻=261.2.

### Step 2: (4-bromo-3-fluoro-1,2-phenylene)dimethanol

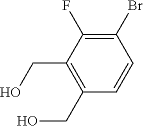 [SMILES: OCc1ccc(Br)c(F)c1CO]

To a solution of 4-bromo-3-fluorophthalic acid (30 g, 114.5 mmol) in THF (600 mL) was added BH₃-THF (343.5 mL, 343.5 mmol) at 0° C. The resulting solution was stirred for overnight at 50° C. The reaction was then quenched with MeOH (350 mL) at 0° C. The resulting mixture was concentrated under vacuum. The residue was purified by combi-flash (DCM/MeOH=4:1) to give the product (23 g, 85.8%). LCMS (ESI) m/z [M−H]⁻=233.

### Step 3: 1-bromo-3,4-bis(chloromethyl)-2-fluorobenzene

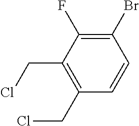 [SMILES: Fc1c(Br)ccc(CCl)c1CCl]

To a solution of (4-bromo-3-fluoro-1,2-phenylene)dimethanol (23 g, 98.2 mmol) in SOCl₂ (100 mL) at 0° C. The resulting solution was stirred for 3 h at 80° C. The resulting mixture was concentrated under vacuum. The reaction was then quenched with ice-water (150 mL). The mixture was acidified to PH >8 with NaOH solution. The resulting solution was extracted with 3-200 mL of DCM. The resulting mixture was concentrated under vacuum. The residue was the crude product (14 g, 52%).

### Step 4: 5-bromo-4-fluoro-1,3-dihydrobenzo[c]thiophene

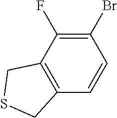 [SMILES: Fc1c(Br)ccc2c1CSC2]

To a solution of 1-bromo-3,4-bis(chloromethyl)-2-fluorobenzene (14 g, 51.8 mmol) in EtOH (100 mL) was added Na₂S·9H₂O (19.3 g, 80.3 mmol) and H₂O (20 mL). The resulting solution was stirred at 80° C. for overnight. The resulting mixture was concentrated under vacuum. The resulting mixture was quenched with H₂O (100 mL). The resulting solution was extracted with 3-100 mL of DCM. The organic layer was washed with 3×60 mL brine. The residue was purified by combi-flash (EtOAc/PE=2%:98%) to give the product (2.9 g, 24.1%).

### Step 5: 5-bromo-4-fluoro-1,3-dihydrobenzo[c]thiophene 2,2-dioxide

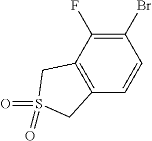 [SMILES: O=S1(=O)Cc2ccc(Br)c(F)c2C1]

To a solution of 5-bromo-4-fluoro-1,3-dihydrobenzo[c]thiophene (2.9 g, 12.5 mmol) in THF:H₂O=4:1 (100 mL) were added NalO₄ (8.1 g, 37.5 mmol), RuCl₃ (281 mg, 1.25 mmol) at 0° C. The resulting solution was stirred for 2 h at rt. The resulting solution was extracted with 3×50 mL of EtOAc.

The organic layers were concentrated under vacuum. The residue was purified by combi-flash (EtOAc/PE=35:65%) to give the product (2.1 g, 63.6%). ¹H NMR (500 MHz, Methanol-d₄) δ 7.68 (ddt, J=7.5, 6.9, 1.0 Hz, 1H), 7.19 (dd, J=8.0, 1.0 Hz, 1H), 4.52 (s, 2H), 4.48 (d, J=1.0 Hz, 2H).

### Step 6: cis-3-(3-((4-fluoro-2,2-dioxido-1,3-dihydrobenzo[c]thiophen-5-yl)amino)-1H-pyrazol-5-cyclopentyl isopropylcarbamate

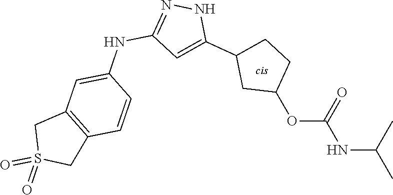 [SMILES: CC(C)NC(=O)OC1CCC(c2cc(Nc3ccc4c(c3)CS(=O)(=O)C4)n[nH]2)C1]

A mixture of 5-bromo-4-fluoro-1,3-dihydrobenzo[c]thiophene 2,2-dioxide (87 mg, 0.33 mmol), cis-3-(3-amino-1H-pyrazol-5-yl)cyclopentyl isopropylcarbamate (65.5 mg, 0.26 mmol). Brettphos Pd G3 (29.9 mg, 0.033 mmol) and K₂CO₃ (135 mg, 0.98 mmol) in t-BuOH (5 mL) was stirred at 110° C. for 16 h under a nitrogen atmosphere. LCMS showed the reaction was complete. The mixture was filtered and the filtrate was concentrated under vacuum. The residue was purified by silica gel column chromatography, eluting with PE/EA (0-100%) to afford the product in a racemic form, which was further separated by Chiral Prep-HPLC to give:

Enantiomer 1 (Example 8a, 100% ee); Retention time: 4.813 min. ¹H NMR (500 MHz, DMSO-d₆) δ 11.81 (s, 1H), 8.20 (s, 1H), 8.08 (t, J=8.5, 1H), 7.05 (d, J=8.5, 1H), 6.94 (d, J=7.5, 1H), 5.75 (s, 1H), 4.99 (m, 1H), 4.51 (s, 2H), 4.42 (s, 2H), 3.58 (m, 1H), 3.12-2.95 (m, 1H), 2.45 (m, 1H), 2.05-1.97 (m, 1H), 1.97-1.85 (m, 1H), 1.70 (m, 2H), 1.58 (m, 1H), 1.03 (m, 6H). LC-MS (ESI): m/z [M+H]⁺=437.35.

Enantiomer 2 (Example 8b, 100% cc); Retention time: 7.178 min. ¹H NMR (500 MHz, DMSO-d₆) δ 11.81 (s, 1H), 8.20 (s, 1H), 8.08 (t, J=8.5, 1H), 7.05 (d, J=8.5, 1H), 6.94 (d, J=7.5, 1H), 5.75 (s, 1H), 4.99 (m, 1H), 4.51 (s, 2H), 4.42 (s, 2H), 3.58 (m, 1H), 3.12-2.95 (m, 1H), 2.45 (m, 1H), 2.05-1.97 (m, 1H), 1.97-1.85 (m, 1H), 1.70 (m, 2H), 1.58 (m, 1H), 1.03 (m, 6H). LC-MS (EST): m/z [M+H]⁺=437.35.

Chiral analytical method: Column: CHIRALPAK IE 4.6×250 mm 5 μm; Mobile phase: A for MtBE (0.1% 2M NH₃ MeOH) and B for MeOH:DCM=50:50 (v/v); Gradient: Mobile Phase A:Mobile Phase B=20:80 (v/v); HPLC Equipment: HPLC-Agilent; Column temperature: 35° C.

Chiral Prep-HPLC Condition: CHIRALPAK IE 20×250 mm 5 μm; Mobile phase: A for MtBE and B for MeOH:DCM-50:50 (0.2% 2M NH₃ MeOH): Gradient: Mobile Phase A:Mobile Phase B=20:80 (v/v); Flow Rate: 18 mL/min, Wavelength: UV 270 nm and 280 nm. Prep-HPLC Equipment: Prep-HPLC-Gilson; Column temperature: 25° C.

### Example 9: cis-3-(3-((4-fluoro-2,2-dioxido-1,3-dihydrobenzo[c]thiophen-5-yl)amino)-1H-pyrazol-5-yl)cyclopentyl propylcarbamate

### Step 1: cis-benzyl (5(3-hydroxycyclopentyl)-1H-pyrazol-3-yl)carbamate

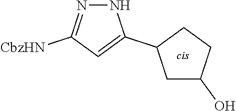 [SMILES: O=C(Nc1cc(C2CCC(O)C2)[nH]n1)OCc1ccccc1]

To a round-bottom flask charged with cis-benzyl (1-(tert-butyl)-3-(3-hydroxycyclopentyl)-1H-pyrazol-5-yl)carbamate (5 g, 13.98 mmol) was added 100 mL formic acid. The resulting mixture was stirred at 75° C. for over night. The solvent was removed under vacuum to yield crude product, which was purified by silica gel column chromatography, eluting with EA/PE (0-75%) to afford the product (3.5 g, 70%). LC-MS (ESI): m/z [M+H]⁺=302.3.

### Step 2: cis-3-(3-amino-1H-pyrazol-5-yl)cyclopentan-1-ol

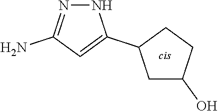 [SMILES: Nc1cc(C2CCC(O)C2)[nH]n1]

To a solution of cis-benzyl (5-(3-hydroxycyclopentyl)-1H-pyrazol-3-yl)carbamate (3.5 g, 11.6 mmol) in THF (100 mL) was added Pd/C (10%, wet, 1 g). The suspension was degassed and purged with hydrogen 3 times. The resulting mixture was stirred at room temperature under a hydrogen balloon for 3 h. The mixture was filtered, and the filter cake was washed with EA (50 mL×3). The filtrate was concentrated under reduced pressure to afford the crude product, which was directly used for the next step without further purification. LC-MS (ESI): m/z [M+H]⁺=168.3.

### Step 3: cis-4-fluoro-54(5-(3-hydroxycyclopentyl)-1H-pyrazol-3-yl)amino)-1,3-dihydrobenzo[c]thiophene 2,2-dioxide

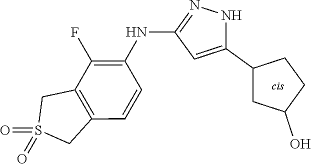 [SMILES: O=S1(=O)Cc2ccc(Nc3cc(C4CCC(O)C4)[nH]n3)c(F)c2C1]

A mixture of 5-bromo-4-fluoro-1,3-dihydrobenzo[c]thiophene 2,2-dioxide (1 g, 3.774 mmol), cis-3-(3-amino-1H-pyrazol-5-yl)cyclopentan-1-ol (951 mg, 3.774 mmol), Brettephos Pd G3 (341 mg, 0.377 mmol) and K₂CO₃ (1.56 g, 11.322 mmol) in t-BuOH (400 mL) was stirred for 3 h at 100° C. in the sealed tube under N₂ atmosphere. The solution was concentrated under reduced pressure. The residue was purified by silica gel column chromatography, eluting with PE/EtOAc (1:5) to afford the product (1.2 g, 90%). LC-MS (ESI): m/z [M+H]⁺=353.2.

### Step 4: cis-4-nitrophenyl 3-((4-fluoro-2,2-dioxido-1,3-dihydrobenzo[c]thiophen-5-yl)amino)-5-(3-(((4-nitrophenoxy)carbonyl)oxy)cyclopentyl)-1H-pyrazole-1-carboxylate

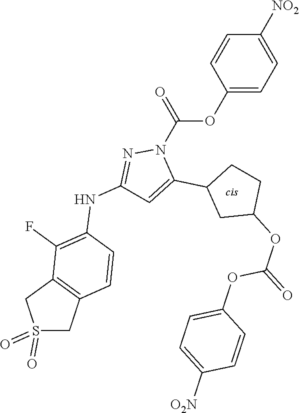 [SMILES: O=C(Oc1ccc([N+](=O)[O-])cc1)OC1CCC(c2cc(Nc3ccc4c(c3F)CS(=O)(=O)C4)nn2C(=O)Oc2ccc([N+](=O)[O-])cc2)C1]

A mixture of cis-4-fluoro-5-((5-(3-hydroxycyclopentyl)-1H-pyrazol-3-yl)amino)-1,3-dihydrobenzo[c]thiophene 2,2-dioxide (1.2 g, 3.399 mmol). DMAP (83 mg, 0.68 mmol), pyridine (1.6 g, 20.394 mmol) and 4-nitrophenyl carbonochloridate (2.05 g, 10.197 mmol) in DCM (50 mL) was stirred for 16 h at 45° C. The resulting mixture was concentrated under reduced pressure. The residue was purified by silica gel column chromatography, eluting with PE/EtOAc (1:3) to afford the product (1.2 g, 51%), LC-MS (ESI): m/z [M+H]⁺=682.4.

### Step 5: cis-3-(3-((4-fluoro-2,2-dioxido-1,3-dihydrobenzo[c]thiophen-5-yl)amino)-1H-pyrazol-5-yl)cyclopentyl propylcarbamate

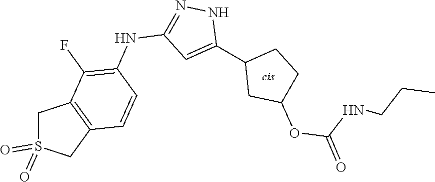 [SMILES: CCCNC(=O)OC1CCC(c2cc(Nc3ccc4c(c3F)CS(=O)(=O)C4)n[nH]2)C1]

A mixture of cis-4-nitrophenyl 3-((4-fluoro-2,2-dioxido-1,3-dihydrobenzo[c]thiophen-5-yl)amino)-5-(3-(((4-nitrophenoxy)carbonyl)oxy)cyclopentyl)-1H-pyrazole-1-carboxylate (70 mg, 0.102 mmol), propan-1-amine (18 mg, 0.306 mmol) and DIEA (155 mg, 1.2 mmol) in THF (5 mL) was stirred for 16 h at 50° C. The mixture was concentrated under reduced pressure. The residue was purified by silica gel column chromatography, eluting with PE/EtOAc (1:10) to afford the crude product. The crude product was further purified by prep HPLC (Waters SunFire C18: RD-CO-058 column, eluting with a gradient of acetonitrile/water containing 0.1% FA, 10%-60%) to afford the product in a racemic form, which was further separated by Chiral Prep-HPLC to give:

Enantiomer 1 (Example 9a, 100% ee); Retention time: 5.39 min. ¹H NMR (500 MHz, DMSO-d₆) δ 11.81 (s, 1H), 8.20 (s, 1H), 8.08 (t, J=8.2 Hz, 1H), 7.05 (d, J=8.7 Hz, 2H), 5.75 (s, 1H), 5.05-4.95 (m, 1H), 4.51 (s, 2H), 4.42 (s, 2H), 3.10-2.99 (m, 1H), 2.91 (dd, J=13.1, 6.6 Hz, 2H), 2.48-2.42 (m, 1H), 2.06-1.97 (m, 1H), 1.95-1.85 (m, 1H), 1.78-1.66 (m, 2H), 1.64-1.55 (m, 1H), 1.45-1.33 (m, 2H), 0.82 (t, J=7.4 Hz, 3H). LC-MS (ESI): m/z [M+H]⁺=437.34.

Enantiomer 2 (Example 9b, 100% ee): Retention time: 8.42 min. ¹H NMR (500 MHz, DMSO-d₆) δ 11.81 (s, 1H), 8.20 (s, 1H), 8.08 (t, J=8.2 Hz, 1H), 7.05 (d, J=8.7 Hz, 2H), 5.75 (s, 1H), 5.05-4.95 (m, 1H), 4.51 (s, 2H), 4.42 (s, 2H), 3.10-2.99 (m, 1H), 2.91 (dd, J=13.1, 6.6 Hz, 2H), 2.48-2.42 (m, 1H), 2.06-1.97 (m, 1H), 1.95-1.85 (m, 1H), 1.78-1.66 (m, 2H), 1.64-1.55 (m, 1H), 1.45-1.33 (m, 2H), 0.82 (t, J=7.4 Hz, 3H). LC-MS (ESI): m/z [M+H]⁺=437.34.

Chiral analytical method: Column: CHIRALPAK IE 4.6 mm×250 mm, 5 μm; Mobile phase: A for Hexane and B for EtOH (0.1% 2M NH₃ MeOH); Gradient: Mobile Phase A:Mobile Phase B=20:80 (v/v); HPLC Equipment: HPLC-Agilent. Back pressure: 100 bar; Column temperature: 35° C.

Chiral Prep-HPLC Condition: CHIRALPAK IE 20 mm-250 mm, 5 μm; Mobile phase: A for Hexane and B for EtOH (0.2% 2M NH₃ MeOH): Gradient: Mobile Phase A:Mobile Phase B=20:80 (v/v); Flow Rate: 18 mL/min, Wave Length: UV 200 nm and 270 nm, Prep-HPLC Equipment: Prep-HPLC-Gilson, Back pressure: 100 bar; Column temperature: 25° C.

### Example 10: cis-3-(3-((4-fluoro-2,2-dioxido-1,3-dihydrobenzo[c]thiophen-5-yl)amino)-1H-pyrazol-5-yl)cyclopentyl ((S)-sec-butyl)carbamate

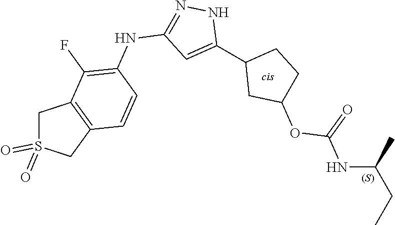

The titled compound was synthesized in the procedures similar to Example 9. ¹H NMR (500 MHz, DMSO-d₆) δ 11.82 (s, 1H), 8.23 (s, 1H), 8.09 (t, J=8.5 Hz, 1H), 7.05 (d, J=8.5 Hz, 1H), 6.91 (d, J=8.3 Hz, 1H), 5.75 (s, 1H), 5.05-4.95 (m, 1H), 4.52 (s, 2H), 4.43 (s, 2H), 3.45-3.35 (m, 1H), 3.10-2.99 (m, 1H), 2.48-2.42 (m, 1H), 2.08-1.% (m, 1H), 1.92-1.85 (m, 1H), 1.78-1.65 (m, 2H), 1.65-1.53 (m, 1H), 1.43-1.30 (m, 2H), 1.05-0.96 (m, 3H), 0.80 (dd, J=13.5, 7.0 Hz, 3H). LC-MS (ESI): m/z [M+H]⁺=451.35.

### Example 11: cis-3-(3-((4-fluoro-2,2-dioxido-1,3-dihydrobenzo[c]thiophen-5-yl)amino)-1H-pyrazol-5-yl)cyclopentyl (1-methylcyclopropyl)carbamate

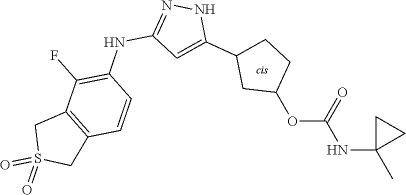 [SMILES: CC1(NC(=O)OC2CCC(c3cc(Nc4ccc5c(c4F)CS(=O)(=O)C5)n[nH]3)C2)CC1]

The titled compound was synthesized in the procedures similar to Example 9. ¹H NMR (500 MHz, DMSO-d₆) δ 11.81 (s, 1H), 8.22 (s, 1H), 8.09 (s, 1H), 7.36 (s, 1H), 7.05 (d, J=8.5 Hz, 1H), 5.74 (s, 1H), 510-4.90 (m, 1H), 4.51 (s, 2H), 4.43 (s, 2H), 3.10-2.95 (m, 1H), 2.48-2.41 (m, 1H), 2.08-1.96 (m, 1H), 1.94-1.85 (m, 1H), 1.80-1.63 (m, 2H), 1.62-1.50 (m, 1H), 1.23 (s, 3H), 0.65-0.55 (m, 2H), 0.50-0.42 (m, 2H). LC-MS (ESI): m/z [M+H]⁺=449.33.

### Example 12: cis-3-(3-((4-fluoro-2,2-dioxido-1,3-dihydrobenzo[c]thiophen-5-yl)amino)-1H-pyrazol-5-yl)cyclopentyl cyclobutylcarbamate

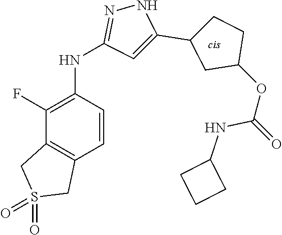 [SMILES: O=C(NC1CCC1)OC1CCC(c2cc(Nc3ccc4c(c3F)CS(=O)(=O)C4)n[nH]2)C1]

The titled compound was synthesized in the procedures similar to Example 9. ¹H NMR (500 MHz, DMSO-d₆) δ 11.81 (s, 1H), 8.20 (s, 1H), 8.09 (t, J=8.6 Hz, 1H), 7.36 (d, J=7.8 Hz, 1H), 7.05 (d, J=8.4 Hz, 1H), 5.75 (s, 1H), 5.08-4.90 (m, 1H), 4.51 (s, 2H), 4.42 (s, 2H), 4.05-3.75 (m, 1H), 3.13-2.97 (m, 1H), 2.49-2.41 (m, 1H), 2.18-1.97 (m, 3H), 1.95-1.81 (m, 3H), 1.80-1.62 (m, 2H), 1.61-1.45 (m, 3H). LC-MS (ESI): m/z [M+H]⁺=449.31.

### Example 13: cis-3-(3-((4-fluoro-2,2-dioxido-1,3-dihydrobenzo[c]thiophen-5-yl)amino)-1H-pyrazol-5-yl)cyclopentyl (3-methyltetrahydrofuran-3-yl)carbamate

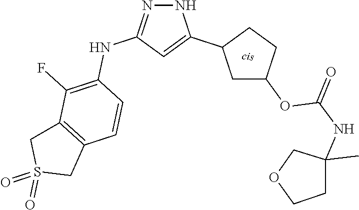 [SMILES: CC1(NC(=O)OC2CCC(c3cc(Nc4ccc5c(c4F)CS(=O)(=O)C5)n[nH]3)C2)CCOC1]

The titled compound was synthesized in the procedures similar to Example 9 in a racemic form, which was further separated by chiral Prep-HPLC to give:

Enantiomer 1 (Example 13a, 100% ee); Retention time: 1.09 min. ¹H NMR (500 MHz, DMSO-d₆) δ 11.81 (s, 1H), 8.20 (s, 1H), 8.08 (s, 1H), 7.23 (s, 1H), 7.05 (d, J=8.5 Hz, 1H), 5.75 (s, 1H), 4.99 (s, 1H), 4.51 (s, 2H), 4.42 (s, 2H), 3.82 (d, J=7.3 Hz, 1H), 3.80-3.69 (m, 2H), 3.43 (d, J=8.6 Hz, 1H), 3.09-2.97 (m, 1H), 2.48-2.41 (m, 1H), 2.20-2.10 (m, 1H), 2.05-1.97 (m, 1H), 1.98-1.82 (m, 1H), 1.80-1.67 (m, 3H), 1.65-1.53 (m, 1H), 1.32 (s, 3H). LC-MS (ESI): m/z [M+H]⁺=479.2.

Enantiomer 2 (Example 13b, 100% ee): Retention time: 2.14 min. ¹H NMR (500 MHz, DMSO-d₆) δ 11.81 (s, 1H), 8.20 (s, 1H), 8.08 (s, 1H), 7.23 (s, 1H), 7.05 (d, J=8.5 Hz, 1H), 5.75 (s, 1H), 4.99 (s, 1H), 4.51 (s, 2H), 4.42 (s, 2H), 3.82 (d, J=7.3 Hz, 1H), 3.80-3.69 (m, 2H), 3.43 (d, J=8.6 Hz, 1H), 3.09-2.97 (m, 1H), 2.48-2.41 (m, 1H), 2.20-2.10 (m, 1H), 2.05-1.97 (m, 1H), 1.98-1.82 (m, 1H), 1.80-1.67 (m, 3H), 1.65-1.53 (m, 1H), 1.32 (s, 3H). LC-MS (ESI): m/z [M+H]⁺=479.2.

Enantiomer 3 (Example 13c, 99.97% ee); Retention time: 2.78 min. ¹H NMR (500 MHz, DMSO-d₆) δ 11.81 (s, 1H), 8.20 (s, 1H), 8.08 (s, 1H), 7.23 (s, 1H), 7.05 (d, J=8.5 Hz, 1H), 5.75 (s, 1H), 4.99 (s, 1H), 4.51 (s, 2H), 4.42 (s, 2H), 3.82 (d, J=7.3 Hz, 1H), 3.80-3.69 (m, 2H), 3.43 (d, J=8.6 Hz, 1H), 3.09-2.97 (m, 1H), 2.48-2.41 (m, 1H), 2.20-2.10 (m, 1H), 2.05-1.97 (m, 1H), 1.98-1.82 (m, 1H), 1.80-1.67 (m, 3H), 1.65-1.53 (m, 1H), 1.32 (s, 3H). LC-MS (ESI): m/z [M+H]⁺=479.2.

Enantiomer 4 (Example 13d, 100% ee): Retention time: 10.56 min. ¹H NMR (500 MHz, DMSO-d₆) δ 11.81 (s, 1H), 8.20 (s, 1H), 8.08 (s, 1H), 7.23 (s, 1H), 7.05 (d, J=8.5 Hz, 1H), 5.75 (s, 1H), 4.99 (s, 1H), 4.51 (s, 2H), 4.42 (s, 2H), 3.82 (d, J=7.3 Hz, 1H), 3.80-3.69 (m, 2H), 3.43 (d, J=8.6 Hz, 1H), 3.09-2.97 (m, 1H), 2.48-2.41 (m, 1H), 2.20-2.10 (m, 1H), 2.05-1.97 (m, 1H), 1.98-1.82 (m, 1H), 1.80-1.67 (m, 3H), 1.65-1.53 (m, 1H), 1.32 (s, 3H). LC-MS (ESI): m/z [M+H]⁺=479.2.

Chiral analytical method: Column: CHIRALPAK IA 4.6 mm×250 mm, 5 μm: Mobile phase: A for MtBE and B for DCM:MeOH: Gradient: Mobile Phase A:Mobile Phase B=60:40 (v/v); HPLC Equipment: HPLC-Agilent, Back pressure: 100 bar; Column temperature: 25° C.

Chiral Prep-HPLC Condition: CHIRALPAK IA 20 mm×250 mm, 5 μm; Mobile phase: A for MtBE and B for DCM:MeOH; Gradient: Mobile Phase A:Mobile Phase B=60:40 (v/v): HPLC Equipment: HPLC-Agilent, Back pressure: 100 bar; Column temperature: 20° C.

### Example 14: cis-3-(3-((4-fluoro-2,2-dioxido-1,3-dihydrobenzo[c]thiophen-5-yl)amino)-1H-pyrazol-5-yl)cyclopentyl (1-methoxy-2-methylpropan-2-yl)carbamate

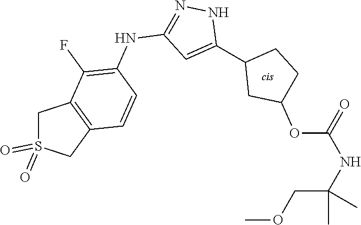 [SMILES: COCC(C)(C)NC(=O)OC1CCC(c2cc(Nc3ccc4c(c3F)CS(=O)(=O)C4)n[nH]2)C1]

The titled compound was synthesized in the procedures similar to Example 9. ¹H NMR (500 MHz, DMSO-d₆) δ 11.80 (s, 1H), 8.21 (s, 1H), 8.09 (t, J=8.6 Hz, 1H), 7.05 (d, J=8.5 Hz, 1H), 6.64 (s, 1H), 5.75 (d, J=1.9 Hz, 1H), 5.05-4.90 (m, 1H), 4.51 (s, 2H), 4.42 (s, 2H), 3.33-3.27 (m, 2H), 3.24 (s, 3H), 3.09-2.99 (m, 1H), 2.48-2.42 (m, 1H), 2.05-1.97 (m, 1H), 1.95-1.84 (m, 1H), 1.78-1.65 (m, 2H), 1.63-1.53 (m, 1H), 1.16 (s, 6H). LC-MS (ESI): m/z [M+H]⁺=481.32.

### Example 15: cis-3-(3-((4-fluoro-2,2-dioxido-1,3-dihydrobenzo[c]thiophen-5-yl)amino)-1H-pyrazol-5-yl)cyclopentyl (3-methyloxetan-3-yl)carbamate

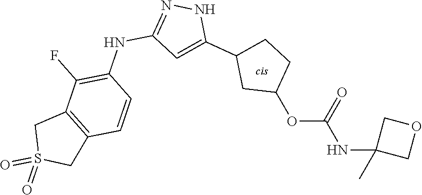 [SMILES: CC1(NC(=O)OC2CCC(c3cc(Nc4ccc5c(c4F)CS(=O)(=O)C5)n[nH]3)C2)COC1]

The titled compound was synthesized in the procedures similar to Example 9 in a racemic form, which was further separated by Chiral Prep-HPLC to give:

Enantiomer 1 (Example 15a, 100% ee); Retention time: 4.74 min. ¹H NMR (500 MHz, DMSO-d₆) δ 11.83 (s, 1H), 8.21 (s, 1H), 8.14-8.02 (m, 1H), 7.62 (s, 1H), 7.03 (d, J=4 Hz, 1H), 5.76 (s, 1H), 5.07-4.97 (m, 1H), 4.61-4.53 (m, 2H), 4.51 (s, 2H), 4.42 (s, 2H), 4.28-4.19 (m, 2H), 3.11-3.00 (m, 1H), 2.48-2.43 (m, 1H), 2.07-1.98 (m, 1H), 1.96-1.85 (m, 1H), 1.80-1.66 (m, 2H), 1.65-1.57 (m, 1H), 1.47 (s, 3H). LC-MS (ESI): m/z [M+H]⁺=465.2.

Enantiomer 2 (Example 15b, 100% ee); Retention time: 6.54 min. ¹H NMR (500 MHz, DMSO-d₆) δ 11.83 (s, 1H), 8.21 (s, 1H), 8.13-8.02 (m, 1H), 7.62 (s, 1H), 7.07 (d, J=4 Hz, 1H), 5.76 (s, 1H), 5.07-4.98 (m, 1H), 4.61-4.53 (m, 2H), 4.51 (s, 2H), 4.42 (s, 2H), 4.28-4.20 (m, 2H), 3.11-3.02 (m, 1H), 2.48-2.41 (m, 1H), 2.07-1.99 (m, 1H), 1.97-1.85 (m, 1H), 1.80-1.65 (m, 2H), 1.65-1.56 (m, 1H), 1.47 (s, 3H). LC-MS (ESI): m/z [M+H]⁺=465.2.

Chiral analytical method: Column: CHIRALPAK IE 4.6 mm×250 mm, 5 μm; Mobile phase: A for MtBE (0.1% 2M NH₃ MeOH) and B for DCM:MeOH=50:50 (v/v); Gradient: Mobile Phase A:Mobile Phase B=50:50 (v/v); HPLC Equipment: HPLC-Agilent, Back pressure: 100 bar; Column temperature: 35° C.

Chiral Prep-HPLC Condition: CHIRALPAK IE 21.2 mm-250 mm, 5 μm; Mobile phase: A for MtBE and B for DCM:MeOH=50:50 (v/v) (0.2% 2M NH₃ MeOH); Gradient: Mobile Phase A:Mobile Phase B=50:50 (v/v); Flow Rate: 18 mL/min, Wave Length: UV 270 nm and 320 nm, Prep-HPLC Equipment: Prep-HPLC-Gilson, Back pressure: 100 bar; Column temperature: 25° C.

### Example 16: cis-3-(3-((4-fluoro-2,2-dioxido-1,3-dihydrobenzo[c]thiophen-5-yl)amino)-1H-pyrazol-5-yl)cyclopentyl (3,3,3-trifluoropropyl)carbamate

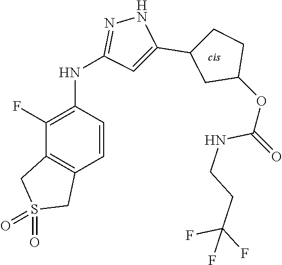 [SMILES: O=C(NCCC(F)(F)F)OC1CCC(c2cc(Nc3ccc4c(c3F)CS(=O)(=O)C4)n[nH]2)C1]

The titled compound was synthesized in the procedures similar to Example 9. ¹H NMR (500 MHz, DMSO-d₆) δ 11.83 (s, 1H), 8.20 (d, J=2.0 Hz, 1H), 8.06 (s, 1H), 7.26 (t, J=5.6 Hz, 1H), 7.05 (d, J=8.5 Hz, 1H), 5.75 (s, 1H), 5.10-4.95 (m, 1H), 4.51 (s, 2H), 4.42 (s, 2H), 3.21 (dd., J=13.0, 6.7 Hz, 2H), 3.10-2.99 (m, 1H), 2.48-2.34 (m, 3H), 2.08-1.97 (m 1H), 1.96-1.86 (m, 1H), 1.80-1.65 (m, 2H), 1.65-1.55 (m, 1H). LC-MS (ESI): m/z [M+H]⁺=491.25.

### Example 17: cis-3-(3-((4-fluoro-2,2-dioxido-1,3-dihydrobenzo[c]thiophen-5-yl)amino)-1H-pyrazol-5-yl)cyclopentyl ((1s,4s)-4-hydroxycyclohexyl)carbamate

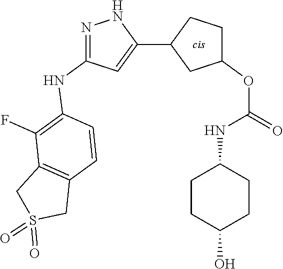 [SMILES: O=C(N[C@H]1CC[C@@H](O)CC1)OC1CCC(c2cc(Nc3ccc4c(c3F)CS(=O)(=O)C4)n[nH]2)C1]

The titled compound was synthesized in the procedures similar to Example 9. ¹H NMR (500 MHz, DMSO-d₆) δ 11.80 (s, 1H), 8.20 (s, 1H), 8.11 (d, J=27.8 Hz, 1H), 7.05 (d, J=8.5 Hz, 1H), 6.98 (d, J=6.8 Hz, 1H), 5.76 (s, 1H), 5.05-4.93 (m, 1H), 4.51 (s, 2H), 4.42 (s, 2H), 4.29 (s, 1H), 3.67 (s, 1H), 3.09-2.98 (m, 1H), 2.47-2.42 (m, 2H), 2.05-1.97 (m, 1H), 1.96-1.85 (m, 1H), 1.78-1.67 (m, 2H), 1.63-1.52 (m, 5H), 1.47-1.35 (m, 4H). LC-MS (ESI): m/z [M+H]⁺=493.3.

### Example 18: cis-3-(3-((4-fluoro-2,2-dioxido-1,3-dihydrobenzo[c]thiophen-5-yl)amino)-1H-pyrazol-5-yl)cyclopentyl ethylcarbamate

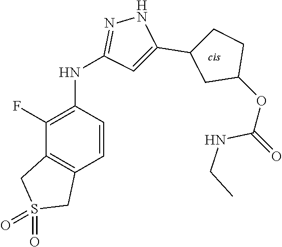 [SMILES: CCNC(=O)OC1CCC(c2cc(Nc3ccc4c(c3F)CS(=O)(=O)C4)n[nH]2)C1]

The tided compound was synthesized in the procedures similar to Example 9 in a racemic form, which was further separated by chiral Prep-HPLC to give:

Enantiomer 1 (Example 18a, 100% ee). ¹H NMR (500 MHz, DMSO-d₆) δ 11.81 (s, 1H), 8.20 (s, 1H), 8.08 (t, J=8.2 Hz, 1H), 7.17-6.97 (m, 2H), 5.75 (s, 1H), 5.00 (s, 1H), 4.51 (s, 2H), 4.42 (s, 2H), 3.10-3.01 (m, 1H), 2.98 (dt, J=13.6, 7.0 Hz, 2H), 2.45 (dd, J=14.0, 7.3 Hz, 1H), 2.05-1.97 (m, 1H), 1.96-1.85 (m, 1H), 1.79-1.65 (m, 2H), 1.61 (d, J=13.8 Hz, 1H), 0.99 (t, J=7.2 Hz, 3H). LC-MS (ESI): m/z [M+H]⁺=423.1.

Enantiomer 2 (Example 18b, 100% ee). ¹H NMR (500 MHz, DMSO-d₆) δ 11.81 (s, 1H), 8.20 (s, 1H), 8.08 (t, J=8.2 Hz, 1H), 7.17-6.97 (m, 2H), 5.75 (s, 1H), 5.00 (s, 1H), 4.51 (s, 2H), 4.42 (s, 2H), 3.10-3.01 (m, 1H), 2.98 (dt, J=13.6, 7.0 Hz, 2H), 2.45 (dd., J=14.0, 7.3 Hz, 1H), 2.05-1.97 (m, 1H), 1.96-1.85 (m, 1H), 1.79-1.65 (m, 2H), 1.61 (d, J=13.8 Hz, 1H), 0.99 (t, J=7.2 Hz, 3H). LC-MS (ESI): m/z [M+H]⁺=423.1.

Chiral analytical method: Column: CHIRALPAK IE 4.6 mm×250 mm, 5 μm; Mobile phase: A for MtBE and B for DCM:MeOH=50:50 (v/v) (0.1% 2M NH₃ MeOH); Gradient: Mobile Phase A:Mobile Phase B=40:60 (v/v); HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for MtBE and B for DCM:MeOH=50:50 (v/v) (0.2% 2M NH₃ MeOH); Gradient: Mobile Phase A:Mobile Phase B=40:60 (v/v); Flow Rate: 18 mL/min, Wave Length: UV 270 nm and 280 nm, Prep-HPLC Equipment: Prep-HPLC-Gilson, Back pressure: 100 bar; Column temperature: 25° C.

### Example 19: cis-3-(3-((4-fluoro-2,2-dioxido-1,3-dihydrobenzo[c]thiophen-5-yl)amino)-1H-pyrazol-5-yl)cyclopentyl (1-hydroxy-2-methylpropan-2-yl)carbamate

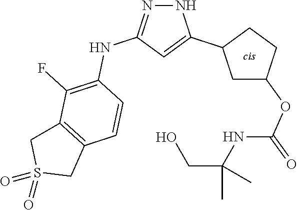 [SMILES: CC(C)(CO)NC(=O)OC1CCC(c2cc(Nc3ccc4c(c3F)CS(=O)(=O)C4)n[nH]2)C1]

The titled compound was synthesized in the procedures similar to Example 9. ¹H NMR (500 MHz, DMSO-d₆) δ 11.81 (s, 1H), 8.20 (d, J=2.0 Hz, 1H), 8.07 (s, 1H), 7.05 (d, J=8.5 Hz, 1H), 6.47 (s, 1H), 5.76 (s, 1H), 5.10-4.90 (m, 1H), 4.70 (t, J=5.6 Hz, 1H), 4.51 (s, 2H), 4.42 (s, 2H), 3.33 (s, 2H), 3.08-2.98 (m, 1H), 2.48-2.41 (m, 1H), 2.05-1.96 (m, 1H), 1.95-1.83 (m, 1H), 1.80-1.64 (m, 2H), 1.64-1.52 (m, 1H), 1.14 (s, 6H). LC-MS (ESI): m/z [M+H]⁺=467.3.

### Example 20: cis-3-(3-((4-fluoro-2,2-dioxido-1,3-dihydrobenzo[c]thiophen-5-yl)amino)-1H-pyrazol-5-yl)cyclopentyl oxetan-3-ylcarbamate

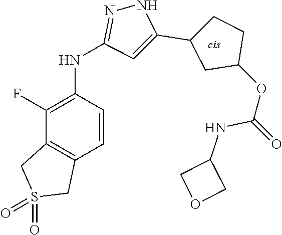 [SMILES: O=C(NC1COC1)OC1CCC(c2cc(Nc3ccc4c(c3F)CS(=O)(=O)C4)n[nH]2)C1]

The titled compound was synthesized in the procedures similar to Example 9. ¹H NMR (500 MHz, DMSO-d₆) δ 11.83 (s, 1H), 8.20 (s, 1H), 8.06 (s, 1H), 7.89 (d, J=5.3 Hz, 1H), 7.05 (d, J=8.5 Hz, 1H), 5.75 (s, 1H), 5.06-4.95 (m, 1H), 4.64 (s, 3H), 4.51 (s, 2H), 4.42 (s, 4H), 3.29 (s, 1H), 3.10-2.99 (m, 1H), 2.48-2.40 (m, 1H), 2.05-1.98 (m, 1H), 2.07-1.98 (m, 1H), 1.79-1.66 (m, 2H), 1.65-1.55 (m, 1H). LC-MS (ESI): m/z [M+H]⁺=451.3.

### Example 21: cis-3-(3-((4-fluoro-2,2-dioxido-1,3-dihydrobenzo[c]thiophen-5-yl)amino)-1H-pyrazol-5-yl)cyclopentyl methyl(tetrahydrofuran-3-yl)carbamate

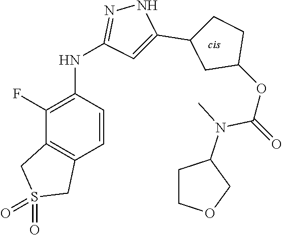 [SMILES: CN(C(=O)OC1CCC(c2cc(Nc3ccc4c(c3F)CS(=O)(=O)C4)n[nH]2)C1)C1CCOC1]

The titled compound was synthesized in the procedures similar to Example 9. ¹H NMR (500 MHz, DMSO-d₆) δ 11.84 (s, 1H), 8.21 (s, 1H), 8.08 (s, 1H), 7.05 (d, J=8.5 Hz, 1H), 5.75 (s, 1H), 5.10-4.95 (m, 1H), 4.80-4.58 (m, 1H), 4.51 (s, 2H), 4.42 (s, 2H), 3.92-3.79 (m, 1H), 3.70-3.59 (m, 2H), 3.58-3.49 (m, 1H), 3.15-3.06 (m, 1H), 2.71 (s, 3H), 2.48-2.38 (m, 1H), 2.12-1.98 (m, 2H), 1.96-1.86 (m, 1H), 1.83-1.67 (m, 4H). LC-MS (ESI): m/z [M+H]⁺=479.32.

### Example 22: cis-3-(3-((4-fluoro-2,2-dioxido-1,3-dihydrobenzo[c]thiophen-5-yl)amino)-1H-pyrazol-5-yl)cyclopentyl (2,2,2-trifluoroethyl)carbamate

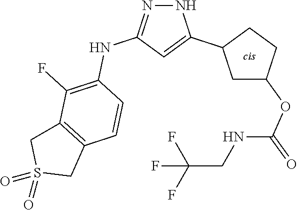 [SMILES: O=C(NCC(F)(F)F)OC1CCC(c2cc(Nc3ccc4c(c3F)CS(=O)(=O)C4)n[nH]2)C1]

The titled compound was synthesized in the procedures similar to Example 9. ¹H NMR (500 MHz, DMSO-d₆) δ 11.82 (s, 1H), 8.20 (s, 1H), 8.08 (s, 1H), 7.87 (t, J=6.3 Hz, 1H), 7.05 (d, J=8.5 Hz, 1H), 5.76 (s, 1H), 5.15-4.95 (m, 1H), 4.51 (s, 2H), 4.42 (s, 2H), 3.83-3.70 (m, 2H), 3.11-3.01 (m, 1H), 2.49-2.45 (m, 1H), 2.08-1.98 (m, 1H), 1.97-1.86 (m, 1H), 1.81-1.67 (m, 2H), 1.66-1.56 (m, 1H). LC-MS (ESI): m/z [M+H]⁺=477.25.

### Example 23: cis-3-(3-((4-fluoro-2,2-dioxido-1,3-dihydrobenzo[c]thiophen-5-yl)amino)-1H-pyrazol-5-yl)cyclopentyl cyclopentylcarbamate

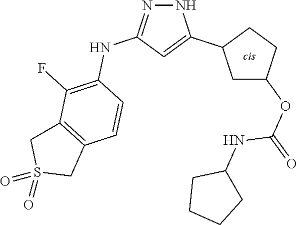 [SMILES: O=C(NC1CCCC1)OC1CCC(c2cc(Nc3ccc4c(c3F)CS(=O)(=O)C4)n[nH]2)C1]

The titled compound was synthesized in the procedures similar to Example 9. ¹H NMR (500 MHz, DMSO-d₆) δ 11.81 (s, 1H), 8.20 (s, 1H), 8.07 (s, 1H), 7.05 (d, J=8.2 Hz, 2H), 5.75 (s, 1H), 5.08-4.90 (m, 1H), 4.51 (s, 2H), 4.42 (s, 2H), 3.82-3.70 (m, 1H), 3.11-2.97 (m, 1H), 2.48-2.40 (m, 1H), 2.08-1.96 (m, 1H), 1.95-1.85 (m, 1H), 1.80-1.65 (m, 4H), 1.65-1.52 (m, 3H), 1.50-1.31 (m, 4H). LC-MS (ESI): m/z [M+H]⁺=463.36.

### Example 24: cis-3-(3-((4-fluoro-2,2-dioxido-1,3-dihydrobenzo[c]thiophen-5-yl)amino)-1H-pyrazol-5-yl)cyclopentyl (tetrahydrofuran-3-yl)carbamate

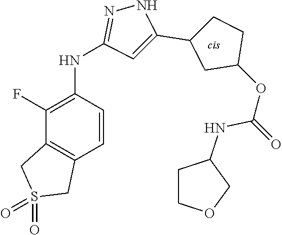 [SMILES: O=C(NC1CCOC1)OC1CCC(c2cc(Nc3ccc4c(c3F)CS(=O)(=O)C4)n[nH]2)C1]

The titled compound was synthesized in the procedures similar to Example 9 in a racemic form, which was further separated by chiral Prep-HPLC to give:

Enantiomer 1 (Example 24a, 99.65% ee); Retention time: 1.187 min. ¹H NMR (500 MHz, DMSO-d₆) δ 11.81 (s, 1H), 8.20 (s, 1H), 8.08 (t, J=8.2 Hz, 1H), 7.17-6.97 (m, 2H), 5.75 (s, 1H), 5.00 (s, 1H), 4.51 (s, 2H), 4.42 (s, 2H), 3.10-3.01 (m, 1H), 2.98 (dt, J=13.6, 7.0 Hz, 2H), 2.45 (dd, J=14.0, 7.3 Hz, 1H), 2.05-1.97 (m, 1H), 1.96-1.85 (m, 1H), 1.79-1.65 (m, 2H), 1.61 (d, J=13.8 Hz, 1H), 0.99 (t, J=7.2 Hz, 3H). LC-MS (ESI): m/z [M+H]⁺=465.2.

Enantiomer 1 (Example 24b, 99.34% ee); Retention time: 1.545 min. ¹H NMR (500 MHz, DMSO-d₆) δ 11.81 (s, 1H), 8.20 (s, 1H), 8.08 (t, J=8.2 Hz, 1H), 7.17-6.97 (m, 2H), 5.75 (s, 1H), 5.00 (s, 1H), 4.51 (s, 2H), 4.42 (s, 2H), 3.10-3.01 (m, 1H), 2.98 (dt, J=13.6, 7.0 Hz, 2H), 2.45 (dd, J=14.0, 7.3 Hz, 1H), 2.05-1.97 (m, 1H), 1.96-1.85 (m, 1H), 1.79-1.65 (m, 2H), 1.61 (d, J=13.8 Hz, 1H), 0.99 (t, J=7.2 Hz, 3H). LC-MS (ESI): m/z [M+H]⁺=465.2.

Chiral analytical method: Column: CHIRALPAK IA 4.6 mm×250 mm, 5 μm; Mobile phase: A for Hex:DCM and B for IPA; Gradient: Mobile Phase A:Mobile Phase B=50:60 (v/v): HPLC Equipment: HPLC-Agilent, Back pressure: 100 bar; Column temperature: 35° C.

Chiral Prep-HPLC Condition: CHIRALPAK IE 20 mm×250 mm, 5 μm; Mobile phase: A for Hex:DCM and B for IPA; Gradient: Mobile Phase A:Mobile Phase B=50:50 (v/v); Flow Rate: 18 mL/min, Wave Length: UV 270 nm and 280 nm. Prep-HPLC Equipment: Prep-HPLC-Gilson, Back pressure: 100 bar; Column temperature: 25° C.

### Example 25: cis-3-(3-((4-fluoro-2,2-dioxido-1,3-dihydrobenzo[c]thiophen-5-yl)amino)-1H-pyrazol-5-yl)cyclopentyl ((1s,4s)-4-hydroxy-4-methylcyclohexyl)carbamate

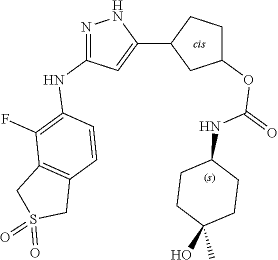 [SMILES: C[C@]1(O)CC[C@@H](NC(=O)OC2CCC(c3cc(Nc4ccc5c(c4F)CS(=O)(=O)C5)n[nH]3)C2)CC1]

The titled compound was synthesized in the procedures similar to Example 9. ¹H NMR (500 MHz, DMSO-d₆) δ 11.80 (s, 1H), 8.20 (s, 1H), 8.07 (d, J=9.0 Hz, 1H), 7.05 (d, J=8.5 Hz, 1H), 6.87 (dd., J=67.2, 8.1 Hz, 1H), 5.75 (s, 1H), 5.10-4.90 (m, 1H), 4.51 (s, 2H), 4.42 (s, 2H), 3.95 (s, 1H). 3.17 (s, 1H), 3.10-2.99 (m, 1H), 2.48-2.38 (m, 1H), 2.05-1.97 (m, 1H), 1.96-1.84 (m, 1H), 1.78-1.65 (m, 2H), 1.64-1.40 (m, 7H), 1.32-1.20 (m, 2H), 1.07 (s, 3H). LC-MS (ESI): m/z [M+H]⁺=507.30.

### Example 26: cis-3-(3-((4-fluoro-2,2-dioxido-1,3-dihydrobenzo[c]thiophen-5-yl)amino)-1H-pyrazol-5-yl)cyclopentyl ((1r,4r)-4-hydroxycyclohexyl)carbamate

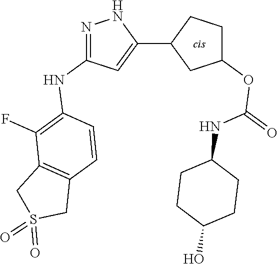 [SMILES: O=C(N[C@H]1CC[C@H](O)CC1)OC1CCC(c2cc(Nc3ccc4c(c3F)CS(=O)(=O)C4)n[nH]2)C1]

The titled compound was synthesized in the procedures similar to Example 9. ¹H NMR (500 MHz, DMSO-d₆) δ 11.81 (s, 1H), 8.20 (s, 1H), 8.08 (s, 1H), 7.05 (d, J=8.2 Hz, 1H), 6.94 (s, 1H), 5.75 (s, 1H), 5.06-4.90 (m, 1H), 4.51 (s, 3H), 4.42 (s, 2H), 3.19 (s, 1H), 3.03 (s, 1H), 2.58-2.54 (m, 1H), 2.48-2.40 (m, 1H), 2.08-1.97 (m, 1H), 1.96-1.87 (m, 1H), 1.83-1.66 (m, 6H), 1.63-1.52 (m, 1H), 1.25-1.08 (m, 4H). LC-MS (ESI): m/z [M+H]⁺=493.29.

### Example 27: cis-3-(3-((6-fluoro-2,2-dioxido-1,3-dihydrobenzo[c]thiophen-5-yl)amino)-1H-pyrazol-5-yl)cyclopentyl isopropylcarbamate

### Step 1: 1-bromo-4,5-bis(bromomethyl)-2-fluorobenzene

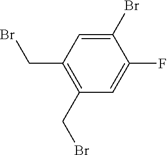 [SMILES: Fc1cc(CBr)c(CBr)cc1Br]

A mixture of 1-bromo-2-fluoro-4-dim-ethylbenzene (5 g, 24.6 mmol). NBS (10.85 g, 60.96 mmol) and AIBN (1.98 g, 12.3 mmol) in CCl₄ (80 mL) was heated to 80° C., and stirred under nitrogen atmosphere for 12 h. The mixture was concentrated under reduced pressure, and the residue was purified by silica gel column chromatography, eluting with PE/EA (50:1) to afford the product (6 g, 67%).

### Step 2: 5-bromo-6-fluoro-1,3-dihydrobenzo[c]thiophene

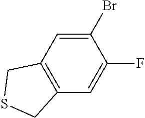 [SMILES: Fc1cc2c(cc1Br)CSC2]

A mixture of 1-bromo-4,5-bis(bromomethyl)-2-fluorobenzene (2 g, 5.54 mmol) and Na₂S·9H₂O (1.6 g, 6.65 mmol) in EtOH (150 mL) was stirred under nitrogen atmosphere at RT for 16 h.

The mixture was concentrated under reduced pressure, and the residue was purified by silica gel column chromatography, eluting with PE/EA (50:1) to afford the product (0.6 g, 46%). ¹H NMR (500 MHz, DMSO-d₆) δ 7.65 (d, J=6.6, 1H), 7.34 (d, J=9.3, 1H), 4.18 (s, 4H).

### Step 3: 5-bromo-6-fluoro-1,3-dihydrobenzo[c]thiophene 2,2-dioxide

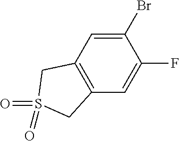 [SMILES: O=S1(=O)Cc2cc(F)c(Br)cc2C1]

A mixture of 5-bromo-6-fluoro-1,3-dihydrobenzo[c]thiophene (600 mg, 2.57 mmol), NalO₄ (1.67 g, 7.72 mmol) and RuCl₃·H₂O (58 mg, 0.26 mmol) in THF (20% H₂O, 10 mL) was stirred under nitrogen atmosphere at RT for 16 h. The solution was extracted with EA (30 mL×2). The combined organic layers were dried over anhydrous Na₂SO₄, filtered, and concentrated under reduced pressure. The residue was purified by silica gel column chromatography, eluting with PE/EA (4:1) to afford the product (0.51 g, 75%). LC-MS (ESI): m/z [M+H]⁺=265.0.

### Step 4: cis-3-(3-((6-fluoro-2,2-dioxido-1,3-dihydrobenzo[c]thiophen-5-yl)amino)-1H-pyrazol-5-ylcyclopentyl isopropylcarbamate

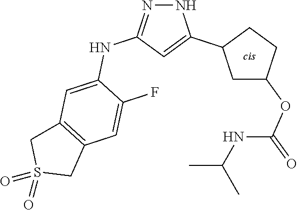 [SMILES: CC(C)NC(=O)OC1CCC(c2cc(Nc3cc4c(cc3F)CS(=O)(=O)C4)n[nH]2)C1]

To a mixture of 5-bromo-6-fluoro-1,3-dihydrobenzo[c]thiophene 2,2-dioxide (53 mg, 0.2 mmol) and cis-3-(3-amino-1H-pyrazol-5-yl)cyclopentyl isopropylcarbamate (50 mg, 0.2 mmol) in t-BuOH (3 mL) was added BrettPhos Pd G3 (18 mg, 0.02 mmol) and K₂CO₃ (84 mg, 0.6 mmol). The reaction mixture was stirred at 110° C. under nitrogen atmosphere for 4 h. The mixture was concentrated under reduced pressure. The residue was purified by prep HPLC (Waters SunFire C18: RD-CO-058 column, eluting with 34%-49% of water (containing 0.1% FA) in acetonitrile to afford the product (13 mg, 15%). ¹H NMR (500 MHz, DMSO-d₆) δ 11.81 (s, 1H), 8.22 (d, J=1.8, 1H), 8.09 (s, 1H), 7.17 (d, J=11.7, 1H), 6.96-6.95 (m, 1H), 5.76 (s, 1H), 5.00 (s, 1H), 4.41 (s, 2H), 4.36 (s, 2H), 3.60-3.56 (m, 1H), 3.08-3.00 (m, 1H), 2.49-2.43 (m, 1H), 2.06-1.87 (m, 2H), 1.79-1.54 (m, 3H), 1.04-1.02 (m, 6H). LC-MS (ESI): m/z [M+H]⁺=437.3.

### Example 28: cis-3-(3-((6-methyl-2,2-dioxido-1,3-dihydrobenzo[c]thiophen-5-yl)amino)-1H-pyrazol-5-yl)cyclopentyl isopropylcarbamate

### Step 1: (4-bromo-5-methyl-1,2-phenylene)dimethanol

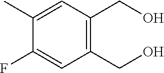

To a solution of 4-bromo-5-methylphthalic acid (11.0 g, 42.64 mmol) in THF (150 mL) was added BH₃-THF (210 mL, 210 mmol) at 0° C. The resulting solution was stirred for 3 hours at 500° C. The reaction was then quenched with MeOH (80 mL) at 0 T. The resulting mixture was concentrated under vacuum. The residue was purified by combi-flash (DCM/MeOH=10:1) to give the product (8.5 g, 86.7%). LCMS (ESI) m/z [M+Na]⁺=252.9.

### Step 2: 1-bromo-4,5-bis(chloromethyl)-2-methylbenzene

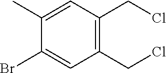

(4-bromo-5-methyl-1,2-phenylene)dimethanol (3.5 g, 15.2 mmol) was dissolved in SOCl₂ (50 mL) at 0° C. The reaction solution was stirred for 3 h at 80° C. The resulting mixture was concentrated under vacuum. The residue was then quenched with ice-water (80 mL). The mixture was adjusted to PH >8 with 1 N NaOH aqueous. The resulting solution was extracted with 3×80 mL of DCM. The combined organic phase was washed with brine, dried over Na₂SO₄, filtered, the filtrated was concentrated, the residue was purified by column chromatography (PE), to give the product (200 mg, 5%).

### Step 3: 5-bromo-6-methyl-1,3-dihydrobenzo[c]thiophene

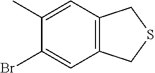

To a solution of 1-bromo-4,5-bis(chloromethyl)-2-methylbenzene (200 mg, 0.75 mmol) in EtOH (50 mL) was added Na₂S·9H₂O (235 mg, 0.98 mmol). The reaction mixture was stirred at 80° C. for overnight. The resulting mixture was concentrated under vacuum. The residue was purified by combi-flash (PE) to give the product (130 mg, 76%).

### Step 4: 5-bromo-6-methyl-1,3-dihydrobenzo[c]thiophene 2,2-dioxide

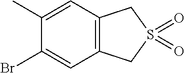

To a solution of 5-bromo-6-methyl-1,3-dihydrobenzo[c]thiophene (130 mg, 0.57 mmol) in THF/H₂O=2 mL/0.5 mL was added NalO₄ (370 mg, 1.72 mmol). RuCl₃ (12 mg, 0.057 mmol) at 0° C. The reaction was stirred for 4 hours at room temperature. The resulting solution was extracted with 3×10 mL with EtOAc/H₂O. The combined organic phase was washed with brine, dried over Na₂SO₄, filtered, the filtrated was concentrated to give crude product (150 mg).

### Step 5: cis-3-(3-((6-methyl-2,2-dioxido-1,3-dihydrobenzo[c]thiophen-5-yl)amino)-1H-pyrazol-5-yl)cyclopentyl isopropylcarbamate

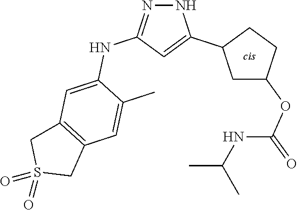 [SMILES: Cc1cc2c(cc1Nc1cc(C3CCC(OC(=O)NC(C)C)C3)[nH]n1)CS(=O)(=O)C2]

To a mixture of 5-bromo-6-methyl-1,3-dihydrobenzo[c]thiophene 2,2-dioxide (52 mg, 0.2 mmol) and cis-3-(3-amino-1H-pyrazol-5-yl)cyclopentyl isopropylcarbamate (50 mg, 0.2 mmol) in t-BuOH (3 mL) was added BrettPhos Pd G3 (18 mg, 0.02 mmol) and K₂CO₃ (84 mg, 0.6 mmol). The reaction mixture was stirred at 110° C. under nitrogen atmosphere for 4 h. The mixture was concentrated under reduced pressure. The residue was purified by prep HPLC (Waters SunFire C18: RD-CO-058 column, eluting with 36%-58% of water (containing 0.1% FA) in acetonitrile to afford the product (20 mg, 23%). ¹H NMR (500 MHz, DMSO-d₆) δ 11.71 (s, 1H), 7.70 (s, 1H), 7.31 (s, 1H), 7.04 (s, 1H), 6.94 (d, J=7.3, 1H), 5.77 (s, 1H), 5.00 (m, 1H), 4.36 (s, 2H), 4.31 (s, 2H), 3.58 (m, 1H), 3.03 (m, 1H), 2.48-2.42 (m, 1H), 2.22 (s, 3H), 2.01 (m, 1H), 1.96-1.84 (m, 1H), 1.69 (m, 2H), 1.61 (m, 1H), 1.12-0.96 (m, 6H). LC-MS (ESI): m/z [M+H]⁺=433.35.

### Example 29: cis-3-(3-((6-fluoro-2-oxido-1,3-dihydrobenzo[c]thiophen-5-yl)amino)-1H-pyrazol-5-yl)cyclopentyl isopropylcarbamate

### Step 1: 5-bromo-6-fluoro-1,3-dihydrobenzo[c]thiophene 2-oxide

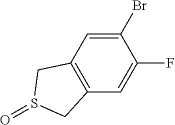 [SMILES: O=S1Cc2cc(F)c(Br)cc2C1]

A mixture of 5-bromo-6-fluoro-1,3-dihydrobenzo[c]thiophene (233 mg, 1 mmol) and NalO₄ (216 mg, 7.72 mmol) in MeOH (50% H₂O, 10 mL) was stirred under nitrogen atmosphere at room temperature for 16 h. The solution was extracted with EA (30 mL×2). The combined organic layers were dried over anhydrous Na₂SO₄, filtered, and concentrated under reduced pressure. The residue was purified by silica gel column chromatography, eluting with PE/EA (9:1) to afford the product (210 mg, 84%). LC-MS (EST): m/z [M+H]⁺=249.3.

### Step 2: cis-3-(3-((6-fluoro-2-oxido-1,3-dihydrobenzo[c]thiophen-5-yl)amino)-1H-pyrazol-5-yl)cyclopentyl isopropylcarbamate

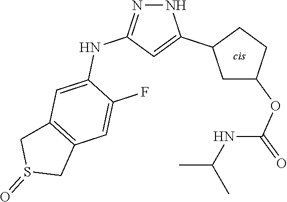 [SMILES: CC(C)NC(=O)OC1CCC(c2cc(Nc3cc4c(cc3F)CS(=O)C4)n[nH]2)C1]

To a mixture of 5-bromo-6-fluoro-1,3-dihydrobenzo[c]thiophene 2-oxide (25 mg, 0.1 mmol) and cis-3-(3-amino-1H-pyrazol-5-yl)cyclopentyl isopropylcarbamate (25 mg, 0.1 mmol) in t-BuOH (3 mL) was added BrettPhos Pd G3 (9 mg, 0.01 mmol) and K₂CO₃ (42 mg, 0.3 mmol). The reaction mixture was stirred at 110° C. under nitrogen atmosphere for 4 h. The mixture was concentrated under reduced pressure. The residue was purified by prep HPLC (Waters SunFire C18: RD-CO-058 column, eluting with 34%-49% of water (containing 0.1% FA) in acetonitrile to afford the product (16 mg, 38%). ¹H NMR (500 MHz, DMSO-d₆) δ 11.78 (s, 1H), 8.08-8.08 (m, 2H), 7.19 (d, J=11.6, 1H), 6.95 (d, J=7.1, 1H), 5.75 (s, 1H), 5.00 (s, 1H), 4.30-4.27 (m, 2H), 3.92 (t, J=15.0, 2H), 3.65-3.52 (m, 1H), 3.14-2.98 (m, 1H), 2.49-2.43 (m, 1H), 2.04-1.87 (m, 2H), 1.77-1.56 (m, 3H), 1.04-1.02 (m, 611). LC-MS (ESI): m/z [M+H]⁺=421.3.

### Example 30: cis-3-(3-((6-fluoro-1,3-dihydrobenzo[c]thiophen-5-yl)amino)-1H-pyrazol-5-yl)cyclopentyl isopropylcarbamate

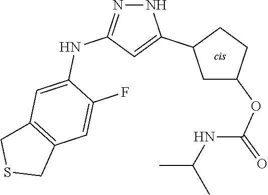 [SMILES: CC(C)NC(=O)OC1CCC(c2cc(Nc3cc4c(cc3F)CSC4)n[nH]2)C1]

To a mixture of 5-bromo-6-fluoro-1,3-dihydrobenzo[c]thiophene (46 mg, 0.2 mmol) and cis-3-(3-amino-1H-pyrazol-5-yl)cyclopentyl isopropylcarbamate (50 mg, 0.2 mmol) in t-BuOH (3 mL) was added BrettPhos Pd G3 (18 mg, 0.02 mmol) and K₂CO₃ (84 mg, 0.6 mmol). The reaction mixture was stirred at 110° C. under nitrogen atmosphere for 4 h. The mixture was concentrated under reduced pressure. The residue was purified by prep HPLC (Waters SunFire C18: RD-CO-058 column, eluting with 34%-49/6 of water (containing 0.1% FA) in acetonitrile to afford the product (22.88 mg, 28%). ¹H NMR (500 MHz, DMSO-d₆) δ 8.00-7.98 (m, 2H), 7.05 (d, J=11.9, 1H), 6.94 (d, J=7.4, 1H), 5.74 (s, 1H), 4.99 (s, 1H), 4.12 (d, J=11.8, 4H), 3.61-3.54 (m, 1H), 3.08-2.99 (m, 1H), 2.48-2.44 (m, 1H), 2.05-1.85 (m, 2H), 1.77-1.55 (m, 3H), 1.04 (d, J=6.3, 6H). LC-MS (ESI): m/z [M+H]⁺=405.3.

### Example 31: cis-3-(3-((4-fluoro-2-oxido-1,3-dihydrobenzo[c]thiophen-5-yl)amino)-1H-pyrazol-5-yl)cyclopentyl isopropylcarbamate

### Step 1: 5-bromo-4-fluoro-1,3-dihydrobenzo[c]thiophene 2-oxide

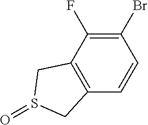 [SMILES: O=S1Cc2ccc(Br)c(F)c2C1]

A mixture of 5-bromo-4-fluoro-1,3-dihydrobenzo[c]thiophene (233 mg, 1 mmol) and NalO₄ (216 mg, 7.72 mmol) in MeOH (50% H₂O, 10 mL) was stirred under nitrogen atmosphere at room temperature for 16 h. The solution was extracted with EA (30 mL×2). The combined organic layers were dried over anhydrous Na₂SO₄, filtered, and concentrated under reduced pressure. The residue was purified by silica gel column chromatography, eluting with PE/EA (9:1) to afford the product (191 mg, 77%). LC-MS (ESI): m/z [M+H]⁺=249.3.

### Step 2: cis-3-(3-((4-fluoro-2-oxido-1,3-dihydrobenzo[c]thiophen-5-yl)amino)-1H-pyrazol-5-yl)cyclopentyl isopropylcarbamate

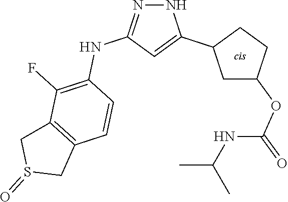 [SMILES: CC(C)NC(=O)OC1CCC(c2cc(Nc3ccc4c(c3F)CS(=O)C4)n[nH]2)C1]

To a mixture of 5-bromo-4-fluoro-1,3-dihydrobenzo[c]thiophene 2-oxide (25 mg, 0.1 mmol) and cis-3-(3-amino-1H-pyrazol-5-yl)cyclopentyl isopropylcarbamate (25 mg, 0.1 mmol) in t-BuOH (3 mL) was added BrettPhos Pd G3 (9 mg, 0.01 mmol) and K₂CO₃ (42 mg, 0.3 mmol). The reaction mixture was stirred at 110° C. under nitrogen atmosphere for 4 h. The mixture was concentrated under reduced pressure. The residue was purified by prep HPLC (Waters SunFire C18: RD-CO-058 column, eluting with 34%-49% of water (containing 0.1% FA) in acetonitrile to afford the product (14.99 mg, 36%). ¹H NMR (500 MHz, DMSO-d₆) 11.76 (s, 1H), 8.08 (s, 1H), 8.06-7.99 (m, 1H), 7.07 (d, J=8.3, 1H), 6.94 (d, J=7.7, 1H), 5.74 (s, 1H), 4.99 (s, 1H), 4.33 (dd, J=33.1, 16.0, 2H), 4.01 (dd, J=19.9, 16.3, 2H), 3.60-3.55 (m, 1H), 3.18-2.95 (m, 1H), 2.47-2.43 (m, 1H), 2.07-1.55 (m, 5H), 1.04 (d, J=6.3, 6H). LC-MS (ESI): m/z [M+H]⁺=421.3.

### Example 32: cis-3-(3-((4-fluoro-2,2-dioxido-1,3-dihydrobenzo[c]thiophen-5-yl)amino)-1H-pyrazol-5-yl)cyclopentyl ethyl(methyl)carbamate

### Step 1: cis-3-(3-amino-1H-pyrazol-5-yl)cyclopentyl ethyl(methyl)carbamate

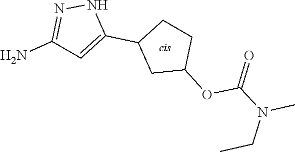 [SMILES: CCN(C)C(=O)OC1CCC(c2cc(N)n[nH]2)C1]

The titled compound was synthesized in the procedures similar to Example 1. LC-MS (ESI): m/z [M+H]⁺=253.2.

### Step 2: cis-3-(34(4-fluoro-2,2-dioxido-1,3-dihydrobenzo[c]thiophen-5-yl)amino)-1H-pyrazol-5-yl)cyclopentyl ethyl(methyl)carbamate

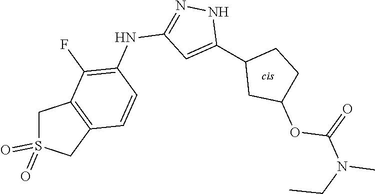 [SMILES: CCN(C)C(=O)OC1CCC(c2cc(Nc3ccc4c(c3F)CS(=O)(=O)C4)n[nH]2)C1]

The titled compound was synthesized in the procedures similar to Example 8. ¹H NMR (500 MHz, DMSO-d₆) δ 11.84 (s, 1H), 8.22 (s, 1H), 8.06 (s, 1H), 7.05 (d, J=7.8 Hz, 1H), 5.75 (s, 1H), 5.10-4.95 (m, 1H), 4.51 (s, 2H), 4.42 (s, 2H), 3.19 (s, 2H), 3.12-3.01 (m, 1H), 2.77 (d, J=2.7 Hz, 3H), 2.46-2.40 (m, 1H), 2.08-1.97 (m, 1H), 1.95-1.85 (m, 1H), 1.82-1.62 (m, 3H), 1.00 (s, 3H). LC-MS (ESI): m/z [M+H]⁺=437.32.

### Example 33: cis-3-(3-((4-fluoro-1,3-dihydrobenzo[c]thiophen-5-yl)amino)-1H-pyrazol-5-yl)cyclopentyl isopropylcarbamate

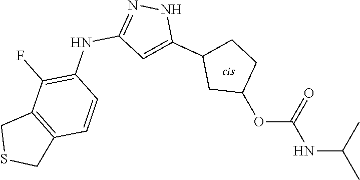 [SMILES: CC(C)NC(=O)OC1CCC(c2cc(Nc3ccc4c(c3F)CSC4)n[nH]2)C1]

The titled compound was synthesized in the procedures similar to Example 30. ¹H NMR (500 MHz, DMSO-d₆) δ 11.73 (s, 1H), 7.96 (d, J=1.9 Hz, 2H), 6.94 (d, J=8.3 Hz, 2H), 5.72 (s, 1H), 4.99 (s, 1H), 4.19 (d, J=20.2 Hz, 4H), 3.62-3.55 (m, 1H), 3.08-2.96 (m, 1H), 2.46-2.41 (m, 1H), 2.05-1.85 (m, 2H), 1.76-1.55 (m, 3H), 1.03 (d, J=6.4 Hz, 6H). LC-MS (ESI): m/z [M+H]⁺=405.3.

### Example 34: cis-3-(3-((4-fluoro-2,2-dioxido-1,3-dihydrobenzo[c]thiophen-5-yl)amino)-1H-pyrazol-5-yl)cyclopentyl ((1r,4r)-4-hydroxy-4-methylcyclohexyl)carbamate

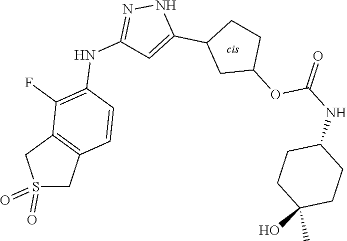 [SMILES: C[C@]1(O)CC[C@H](NC(=O)OC2CCC(c3cc(Nc4ccc5c(c4F)CS(=O)(=O)C5)n[nH]3)C2)CC1]

The titled compound was synthesized in the procedures similar to Example 9. ¹H NMR (500 MHz, DMSO-d₆) δ 11.80 (s, 1H), 8.19 (s, 1H), 8.07 (s, 1H), 7.05 (d, J=8.5 Hz, 1H), 6.94 (d, J=7.6 Hz, 1H), 5.75 (s, 1H), 5.05-4.95 (m, 1H), 4.51 (s, 2H), 4.42 (s, 2H), 4.17 (s, 1H), 3.29 (s, 1H), 3.09-2.97 (m, 1H), 2.48-2.42 (m, 1H), 2.05-1.97 (m, 1H), 1.95-1.85 (m, 1H), 1.78-1.64 (m, 4H), 1.63-1.56 (m, 1H), 1.55-1.46 (m, 2H), 1.39-1.22 (m, 4H), 1.07 (s, 3H). LC-MS (ESI): m/z [M+H]⁺=507.31.

### Example 35: cis-3-(3-((4-methyl-2,2-dioxido-1,3-dihydrobenzo[c]thiophen-5-yl)amino)-1H-pyrazol-5-yl)cyclopentyl isopropylcarbamate

### Step 1: methyl 3-amino-6-iodo-2-methylbenzoate

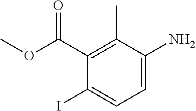

To a solution of methyl 3-amino-2-methylbenzoate (20 g, 121.2 mmol) in DMF (200 mL) was added NIS (28.6 g, 127.3 mmol) at 0° C. The resulting solution was stirred for 2 h at rt. The resulting solution was extracted with 3×200 mL of EtOAc. The combined organic phases were concentrated under reduced pressure. The residue was purified by combi-flash (EtOAc/PE=21%) to give the product (30 g, 85%). LCMS (ESI) m/z [M+H]⁺=292.

### Step 2: 1-ethyl 2-methyl 4-amino-3-methylphthalate

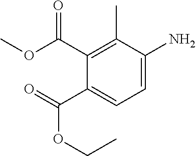 [SMILES: CCOC(=O)c1ccc(N)c(C)c1C(=O)OC]

To a solution of methyl 3-amino-6-iodo-2-methylbenzoate (30 g, 103.1 mmol) in EtOH (300 mL) and DMF (40 mL) were added TEA (31.2 g, 309.3) and Pd(dppf)Cl₂ (6.7 g, 8.2 mmol). The resulting solution was stirred for 5 h at 80° C. under CO atmosphere (3.5 atm). The resulting mixture was concentrated under vacuum. The resulting solution was extracted with 3×300 mL of EtOAc. The resulting mixture was concentrated under vacuum. The residue was purified by combi-flash (EtOAc/PE=7%) to give the product (19.5 g, 79.9%).

### Step 3: 1-ethyl 2-methyl 4-bromo-3-methylphthalate

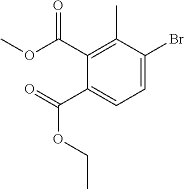 [SMILES: CCOC(=O)c1ccc(Br)c(C)c1C(=O)OC]

To a solution of 1-ethyl 2-methyl 4-amino-3-methylphthalate (19.5 g, 82.3 mmol) in MeCN (300 mL) was added CuBr (17.7 g, 123.5 mmol). Then the tert-Butyl nitrite (25.4 g, 246.9 mmol) was added to the reaction at 0° C. The resulting solution was stirred for 12 h at rt. The resulting mixture was concentrated under vacuum. The residue was purified by combi-flash (EtOAc/PE=11%) to give the product (17.7 g, 71.4%). LCMS (ESI) m/z [M+H]⁺=301.

### Step 4: (4-bromo-3-methyl-1,2-phenylene) dimethanol

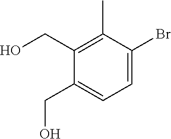

To a solution of 1-ethyl 2-methyl 4-bromo-3-methylphthalate (17.7 g, 58.8 mmol) in THF (260 mL) was added LiBH₄ (123.5 mL, 247 mmol, 2M) at 0° C. The resulting solution was stirred for 30 h at rt. The reaction was then quenched by the addition of MeOH at 0° C. The resulting mixture was concentrated under vacuum. The residue was purified by combi-flash (EtOAc/PE=8%-30%) to give the product (12 g, 88.2%). LCMS (ESI) m/z [M+H]⁺=231.

### Step 5: 1-bromo-3,4-bis(chloromethyl)-2-methylbenzene

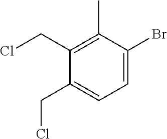

To a solution of (4-bromo-3-methyl-1,2-phenylene) dimethanol (12 g, 51.9 mmol) in SOCl₂ (60 mL) at 0° C. The resulting solution was stirred for 2 h at 80° C. The resulting mixture was concentrated under vacuum. The residue was purified by combi-flash PE to give the product (9 g, 64.7%).

LCMS (ESI) m/z [M+H]⁺=267.

### Step 6: 5-bromo-4-methyl-1,3-dihydrobenzo[c]thiophene

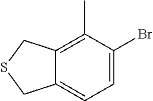

To a solution of 1-bromo-3,4-bis(chloromethyl)-2-methylbenzene (4 g, 14.9 mmol) in EtOH (50 mL) was added Na₂S·9H₂O (5.6 g, 23.1 mmol) and H₂O (10 mL). The resulting solution was stirred at 80° C. for overnight. The solids were filtered out. The resulting mixture was concentrated under vacuum. The residue was purified by combi-flash (EtOAc/PE=5%) to give the product (1.6 g, 47%).

### Step 7: 5-bromo-4-methyl-1,3-dihydrobenzo[c]thiophene 2,2-dioxide

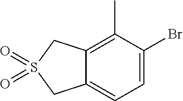

To a solution of 5-bromo-4-methyl-1,3-dihydrobenzo[c]thiophene (1.6 g, 7 mmol) in THF:H₂O=4:1 (24 mL/6 mL) were added NalO₄ (4.5 g, 21 mmol), RuCl₃ (945 mg, 4.2 mmol) at 0° C. The resulting solution was stirred for 5 h at rt. The solids were filtered out. The resulting solution was extracted with 3×30 mL of EtOAc. The organic layers were concentrated under vacuum. The residue was purified by combi-flash (EtOAc/PE=16%) to give the product (1.1 g, 61.1%). ¹H NMR (300 MHz, DMSO-d₆) δ 7.61 (d, J=8.2 Hz, 1H), 7.17 (t, J=7.4 Hz, 1H), 4.57 (s, 2H), 4.50 (d, J=3.9 Hz, 2H), 2.31 (s, 3H).

### Step 8: cis-3-434(4-methyl-2,2-dioxido-1,3-dihydrobenzo[c]thiophen-5-yl)amino)-1H-pyrazol-5-yl)cyclopentyl isopropylcarbamate

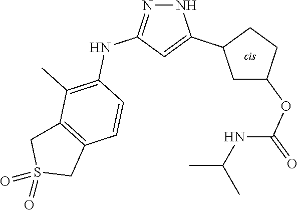 [SMILES: Cc1c(Nc2cc(C3CCC(OC(=O)NC(C)C)C3)[nH]n2)ccc2c1CS(=O)(=O)C2]

To a mixture of 5-bromo-4-methyl-1,3-dihydrobenzo[c]thiophene 2,2-dioxide (52 mg, 0.2 mmol) and cis-3-(3-amino-1H-pyrazol-5-yl)cyclopentyl isopropylcarbamate (50 mg, 0.2 mmol) in t-BuOH (3 mL) was added BrettPhos Pd G3 (18 mg, 0.02 mmol) and K₂CO₃ (84 mg, 0.6 mmol). The reaction mixture was stirred at 110° C. under nitrogen atmosphere for 4 h. The mixture was concentrated under reduced pressure. The residue was purified by prep HPLC (Waters SunFire C18: RD-CO-058 column, eluting with 41%-54% of water (containing 0.1% FA) in acetonitrile to afford the product (11 mg, 13%). ¹H NMR (500 MHz, DMSO-d₆) δ 11.76 (s, 1H), 7.75-7.58 (m, 1H), 7.32 (s, 1H), 7.02 (d, J=10 Hz, 1H), 6.93 (d, J=10 Hz, 1H), 5.72 (s, 1H), 5.03-4.95 (m, 1H), 4.43 (s, 2H), 4.38 (s, 2H), 3.63-3.52 (m, 1H), 3.09-2.98 (m, 1H), 2.48-2.41 (m, 1H), 2.12 (s, 3H), 2.05-1.97 (m, 1H), 1.94-1.85 (m, 1H), 1.77-1.65 (m, 2H), 1.63-1.55 (m, 1H), 1.07-0.97 (m, 6H). LC-MS (ESD): m/z [M+H]⁺=433.2.

### Example 36: CDK2/Cyclin E1 and CDK1/Cyclin B1 Biochemical Assays

Compounds disclosed herein were tested for inhibition of CDK2/Cyclin E1 or CDK1/Cyclin B1 kinase in an assay based on the time-resolved fluorescence-resonance energy transfer (TR-FRET) methodology. The assay was carried out in 384-well low volume black plates in a reaction mixture containing CDK2/Cyclin E1 or CDK1/Cyclin B1, 0.060 mM ATP for CDK2/Cyclin E1 or 0.015 mM ATP for CDK1/Cyclin B1, 0.15 μM Rb (Ser780)-biotin substrate and 0-10 μM compound in buffer containing 50 mM HEPES pH7.0, 0.02% NaN₃, 0.01% BSA, 0.1 mM Orthovanadate, 50 mM MgCl₂, 1 mM DTT and 0.005% Tween-20. The kinase was incubated with compound for 60 minutes at room temperature and the reaction was initiated by the addition of ATP and Rb (Ser780)-biotin substrate. After reaction at room temperature for 120 minutes, an equal volume of stop/detection solution was added according to the manufacture's instruction (Cisbio Bioassays). The stop/detection solution contained Streptavidin-XL665 and Anti-pRb (Ser780) mAb-Eu Cryptate in Detection buffer (Cisbio Bioassays). Plates were incubated at room temperature for 60 minutes, and the TR-FRET signals (ex337 nm, em665 nm/620 nm) were recorded on a PHERAstar FSX plate reader (BMG Labtech). The inhibition percentage of CDK2/Cyclin E1 or CDK1/Cyclin B1 kinase activity in presence of increasing concentrations of compounds was calculated based on the ratio of fluorescence at 665 nm to that at 620 nm. The IC₅₀ for each compound was derived from fitting the data to the four-parameter logistic equation by Dotmatics. The IC₅₀ data as measured for the Examples are shown in Table 1.

TABLE 1

| Example | CDK2/Cyclin E1 IC₅₀ (nM) | CDK1/Cyclin B1 IC₅₀ (nM) |
|---|---|---|
| 1 | 49 | 1139 |
| 2a | 16 | 591 |
| 2b | 4.8 | 207 |
| 3a | 4.1 | 132 |
| 3b | 1.9 | 31 |
| 4 | 17 | 191 |
| 5 | 5 | 117 |
| 6 | 1.1 | 20 |
| 7 | 1.1 | 12 |
| 8a | 0.7 | 30 |
| 8b | 0.5 | 7.9 |
| 9a | 0.8 | 50 |
| 9b | 0.7 | 12 |
| 10 | 0.4 | 5 |
| 11 | 0.3 | 3.3 |
| 14 | 0.6 | 10 |

### Example 37: CDK2/Cyclin E1 and CDK1/Cyclin B1 Biochemical Assays

Compounds disclosed herein were tested for inhibition of CDK2/Cyclin E1 or CDK1/Cyclin B1 kinase in an assay based on the time-resolved fluorescence-resonance energy transfer (TR-FRET) methodology. The assay was carried out in 384-well low volume black plates in a reaction mixture containing CDK2/Cyclin E1 or CDK1/Cyclin B1, 1 mM ATP for CDK2/Cyclin E1 and CDK1/Cyclin B1, 0.15 μM Rb (Ser780)-biotin substrate and 0-10 μM compound in buffer containing 50 mM HEPES pH7.0, 0.02% NaN3, 0.01% BSA, 0.1 mM Orthovanadate, 50 mM MgCl2, 1 mM DTT and 0.005% Tween-20. The kinase was incubated with compound for 60 minutes at room temperature and the reaction was initiated by the addition of ATP and Rb (Ser780)-biotin substrate. After reaction at room temperature for 90 minutes, an equal volume of stop/detection solution was added according to the manufacture's instruction (Cisbio Bioassays). The stop/detection solution contained Streptavidin-XL665 and Anti-pRb (Ser780) mAb-Eu Cryptate in Detection buffer (Cisbio Bioassays). Plates were incubated at room temperature for 60 minutes, and the TR-FRET signals (ex337 nm, em665 nm/620 nm) were recorded on a PHERAstar FSX plate reader (BMG Labtech). The inhibition percentage of CDK2/Cyclin E1 or CDK1/Cyclin B1 kinase activity in presence of increasing concentrations of compounds was calculated based on the ratio of fluorescence at 665 nm to that at 620 nm. The IC50 for each compound was derived from fitting the data to the four-parameter logistic equation by Dotmatics. The IC50 data as measured for the Examples are shown in Table 2.

TABLE 2

| Example | CDK2/Cyclin E1 IC₅₀ (nM) | CDK1/Cyclin B1 IC₅₀ (nM) |
|---|---|---|
| 12 | 1.2 | 609 |
| 13a | 0.5 | 493 |
| 13b | 0.6 | 353 |
| 13c | 0.5 | 208 |
| 13d | 0.9 | 322 |
| 15a | 1.9 | 1087 |
| 15b | 2 | 505 |
| 16 | 0.7 | 155 |
| 17 | 2.3 | 824 |
| 18a | 5.2 | 1300 |
| 18b | 5.2 | 555 |
| 19 | 0.8 | 181 |
| 20 | 31 | 4833 |
| 21 | 24 | 2601 |
| 22 | 4.5 | 1050 |
| 23 | 0.9 | 410 |
| 24a | 17 | 4730 |
| 24b | 61 | 7867 |
| 25 | 1.8 | 768 |
| 26 | 1.5 | 376 |
| 27 | 33 | 7446 |
| 28 | 9.4 | 2899 |
| 29 | 576 | >10000 |
| 30 | 383 | >10000 |
| 31 | 5.3 | 3364 |
| 32 | 4.2 | 493 |
| 33 | 22 | 4883 |
| 34 | 1.1 | 1179 |
| 35 | 0.4 | 169 |

### Example 38: HEK-293 Cell Treatment

HEK-293 cells were seeded at 2000 cells/well at a volume of 50 μl/well in cell culture medium [DMEM, high glucose (Thermo, Cat #11965118), 10% heat-inactive FBS (Gibco, Cat #10091148), 1% PS (Thermo Cat #15140122)] in Corning 96-well plate (Cat #3903), and then incubated overnight. HEK-293 cells were treated with compounds diluted in 0.4% DMSO, dilution was done according to the following protocol: (1) making 250× stock solution in DMSO from 10 mM by 4-fold dilution, total 8 doses were included; (2) making 2 solution in cell culture medium by transferring 1 μl 250× stock solution into 124 μl medium; (3) adding 50 μl of 2× solution to cells for incubation of 72 h.

### Cytotoxicity Detection

25 μl of the CellTiter-Glo® Reagent [(PromegaCat #G9242)] was added to each well in the 96-well plate. The contents were mixed for 2 minutes on an orbital shaker to induce cell lysis. The plate was then allowed to incubate at room temperature for 10 minutes to stabilize luminescent signal. Luminescence was recorded on BMG PheraStar with luminescence protocol.

### IC₅₀ Calculation

The inhibition percentage of the compound was calculated by the following equation: Inhibition percentage of Compound=100−100×(Signal-low control)/(High control-low control), wherein signal=each test compound group; Low control=only medium group (without cells), indicating that cells proliferation is completely inhibited; High control=Cell group with added DMSO and without compound, indicating cells proliferation with no inhibition; Imax is the maximum percentage of inhibition.

The IC₅₀ value of a compound can be obtained by fitting the following equation:

$$Y = Bottom + ( TOP - Bottom ) / ( 1 + ( ( I {C}_{50} / X ) \hat{} hillslope ) )$$

Wherein, X and Y are known values, and IC₅₀, Hillslope, Top and Bottom are the parameters obtained by fitting with software. Y is the inhibition percentage (calculated from the equation), X is the concentration of the compound; IC₅₀ is the concentration of the compound when the 50% inhibition is reached. The smaller the IC₅₀ value is, the stronger the inhibitory ability of the compound is. Vice versa, the higher the IC₅₀ value is, the weaker the ability the inhibitory ability of the compound is; Hillslope represents the slope of the fitted curve, generally around 1*; Bottom represents the minimum value of the curve obtained by data fitting, which is generally 0%±20%; Top represents the maximum value of the curve obtained by data fitting, which is generally 100%±20%. The experimental data were fitted by calculating and analyzing with Dotmatics data analysis software. The IC₅₀ data as measured for the Examples are shown in Table 3.

TABLE 3

| Example | HEK293 IC₅₀ (nM) |
|---|---|
| 1 | >10000.0 |
| 2a | >10000.0 |
| 2b | >10000.0 |
| 3a | 1781.4 |
| 3b | 677 |
| 4 | 8589.4 |
| 5 | 1646.2 |
| 6 | 440 |
| 7 | 683.9 |
| 8a | 513.8 |
| 8b | 462.5 |
| 9a | >10000.0 |
| 9b | 4410.1 |
| 10 | 203.3 |
| 11 | 316.7 |
| 12 | 499.1 |
| 13a | 2145.2 |
| 13b | 558.7 |
| 13c | 1724.4 |
| 13d | 4211.1 |
| 14 | >10000.0 |
| 15a | >10000.0 |
| 15b | 9904.1 |
| 16 | 687.3 |
| 17 | 990.5 |
| 18a | >10000.0 |
| 18b | 522.1 |
| 19 | 201.2 |
| 20 | >10000.0 |
| 21 | >10000.0 |
| 22 | >10000.0 |
| 23 | 2863.9 |
| 24a | >10000.0 |
| 24b | >10000.0 |
| 25 | 1129.5 |
| 26 | 998.4 |
| 27 | >10000.0 |
| 28 | >10000.0 |
| 29 | >10000.0 |
| 30 | >10000.0 |
| 31 | 1571 |
| 32 | >10000 |
| 33 | >10000 |
| 34 | 1428.9 |
| 35 | 739.1 |

What is claimed is:

1. A compound of Formula (I): 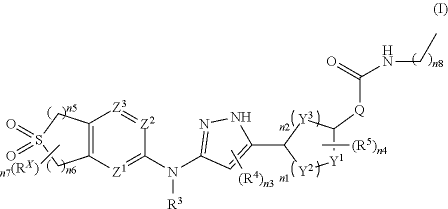
or a N-oxide thereof, or a pharmaceutically acceptable salt thereof, or a stereoisomer thereof, or a tautomer, or a deuterated analog thereof, or a prodrug thereof, wherein:
n1 is 0, 1, or 2;
n2 is 1, 2 or 3;
n3 is 0, 1, or 2;
n4 is 0, 1, 2, 3 or 4;
n5 and n6 are each independently 0, 1 or 2, provided that n5 and n6 are not 0 at the same time;
n7 is 0, 1, 2, 3 or 4;
n8 is 1, 2, 3, or 4;
R^{X} is each independently selected from hydrogen, halogen, —C₁-C₈alkyl, C₃-C₈cycloalkyl, 3- to 8-membered heterocyclyl, C₆-C₁₂aryl, 5- to 12-membered heteroaryl, oxo (═O), —NR^{Xa}R^{Xb}, —OR^{Xa}, —SR^{Xa}, —SO₂R^{Xa}, —SO₂NR^{Xa}R^{Xb}, —C(O)R^{Xa}, —CO₂R^{Xa}, —C(O)NR^{Xa}R^{Xb}, —NR^{Xa}COR^{Xb}, —NR^{Xa}CO₂R^{Xb} or —NR^{Xa}SO₂R^{Xb} or —CN, wherein each of said —C₁-C₈alkyl, C₃-C₈cycloalkyl, 3- to 8-membered heterocyclyl, C₆-C₁₂aryl, or 5- to 12-membered heteroaryl is optionally substituted with at least one R^{Xc}; or
two R^{X} together with the atom(s) to which they are attached, form a 3- to 12-membered ring, said ring comprising 0-3 heteroatoms independently selected from nitrogen, oxygen or optionally oxidized sulfur as ring member(s); said ring is optionally substituted with at least one substituent R^{Xc};
R^{Xa} and R^{Xb} are each independently selected from hydrogen, deuterium, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl, wherein each of said —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl is optionally substituted with at least one substituent R^{Xd};
R^{Xc} and R^{Xd} are each independently selected from hydrogen, deuterium, halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl, 5- to 12-membered heteroaryl, oxo (═O), —NR^{Xe}R^{Xf}, —OR^{Xe}, —SR^{Xe}, —SO₂R^{Xe}, —SO₂NR^{Xe}R^{Xf}, —C(O)R^{Xe}, —CO₂R^{Xe}, —C(O)NR^{Xe}R^{Xf}, —NR^{Xe}COR^{Xf}, —NR^{Xe}CO₂R^{Xf} or —NR^{Xe}SO₂R^{Xf} or —CN;
R^{Xe} and R^{Xf} are each independently selected from —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;
Y¹, Y² and Y³ are each independently selected from O, CH₂ or NH;
Q is selected from 0 or NR^{Q};
R³ and R^{Q} are each independently selected from hydrogen, —C₁-C₈alkyl, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl, wherein each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{3a}; or
R^{3a} is independently selected from hydrogen, halogen, —C₁-C₈alkyl, —C₂-C₈alkenyl, —C₂-C₈alkynyl, —C₃-C₈cycloalkyl, 3- to 8-membered heterocyclyl, —C₆-C₁₂aryl, 5- to 12-membered heteroaryl, oxo (═O), —OR^{3c}, —CN, —SO₂R^{3c}, —SO₂NR^{3c}R^{3d}, —C(O)R^{3c}, —CO₂R^{3c}, —C(O)NR^{3c}R^{3d}, —NR^{3c}R^{3d}, —NR^{3c}COR^{3d}, —NR^{3c}CO₂R^{3d} or —NR^{3c}SO₂R^{3d}, wherein each of —C₁-C₈alkyl, —C₂-C₈alkenyl, —C₂-C₈alkynyl, —C₁-C₈alkoxy, —C₃-C₈cycloalkyl, 3- to 8-membered heterocyclyl, —C₆-C₁₂aryl or 5- to 12-membered heteroaryl is optionally substituted with halogen, —C₁-C₈alkyl, —C₂-C₈alkenyl, —C₂-C₈alkynyl, —C₃-C₈cycloalkyl, 3- to 8-membered heterocyclyl, C₆-C₁₂aryl, 5- to 12-membered heteroaryl, oxo (═O), —CN, —OR^{3e}, —SO₂R^{3e}, —SO₂NR^{3e}R^{3f}, —C(O)R^{3e}, —CO₂R^{3f}, —C(O)NR^{3e}R^{3f}, —NR^{3e}R^{3f}, —NR^{3e}COR^{3f}, —NR^{3e}CO₂R^{3f} or —NR^{3e}SO₂R^{3f};
R^{3c}, R^{3d}, R^{3e} and R^{3f} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;
R⁴ and R⁵ are each independently selected from hydrogen, halogen, —C₁-C₈alkyl, —C₂-C₈alkenyl, —C₂-C₈alkynyl, —C₁-C₈alkoxy, or —C₃-C₈cycloalkyl;
Z¹, Z² and Z³ are each independently selected from —CR^{Z}, or N;
R^{Z}, at each occurrence, is independently selected from hydrogen, halogen, —C₁-C₈alkyl, C₃-C₈cycloalkyl, 3- to 8-membered heterocyclyl, C₆-C₁₂aryl, 5- to 12-membered heteroaryl, —NR^{Za}R^{Zb}, —OR^{Za}, —SR^{Za}, —SO₂R^{Za}, —SO₂NR^{Za}R^{Zb}, —C(O)R^{Za}, —CO₂R^{Za}, —C(O)NR^{Za}R^{Zb}, —NR^{Za}COR^{Zb}, —NR^{Za}CO₂R^{Zb} or —NR^{Za}SO₂R^{Zb} or —CN, wherein each of said —C₁-C₈alkyl, C₃-C₈cycloalkyl, 3- to 8-membered heterocyclyl, C₆-C₁₂aryl, or 5- to 12-membered heteroaryl is optionally substituted with at least one R^{Zc};
R^{Za} and R^{Zb} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl, wherein each of said —C₁-C₈alkyl, —C₂-C₈alkenyl, C₃-C₈alkynyl, C₃-C₈cycloalkyl, 3- to 8-membered heterocyclyl, C₆-C₁₂aryl, or 5- to 12-membered heteroaryl is optionally substituted with at least one substituent R^{Zd};
R^{Zc} and R^{Zd} are each independently selected from halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl, 5- to 12-membered heteroaryl, oxo (═O), —NR^{Ze}R^{Zf}, —OR^{ze}, —SR^{Ze}, —SO₂R^{Ze}, —SO₂NR^{Ze}R^{Zf}, —C(O)R^{Ze}, —CO₂R^{Ze}, —C(O)NR^{Ze}R^{Zf}, —NR^{Ze}COR^{Zf}, —NR^{Ze}COR^{Zf} or —NR^{Ze}SO₂R^{Zf} or —CN;
R^{Ze} and R^{Zf} are each independently selected from —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl.

2. The compound of claim 1, wherein the compound is selected from (IIa) and (IIb): 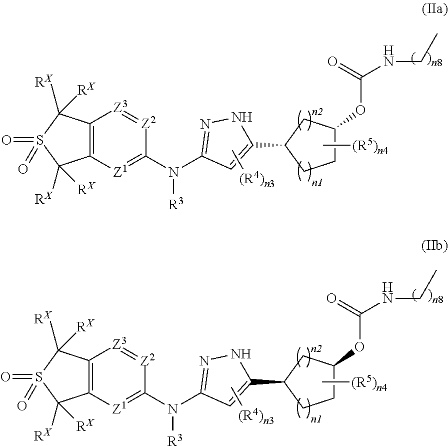
preferably the compound is (IIIa): 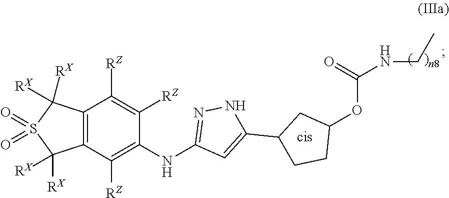
preferably, the compound is selected from (IVa) and (IVb):

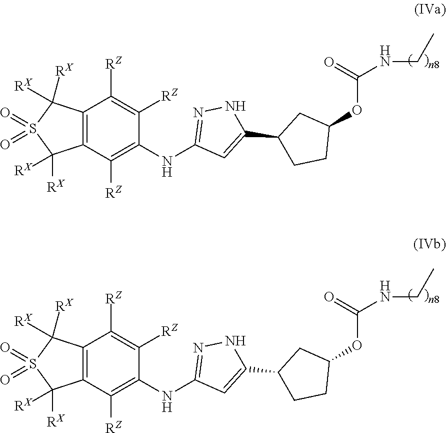

3. The compound of any one of the preceding claims, wherein R³ and R^{Q} are each independently selected from hydrogen, methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, wherein each of said methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{3a};
R^{3a} is independently selected from hydrogen, —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —OR^{3c}, —CN, —SO₂R^{3c}, —SO₂NR^{3c}R^{3d}, —C(O)R^{3c}, —CO₂R^{3c}, —C(O)NR^{3c}R^{3d}, —NR^{3c}R^{3d}, —NR^{3c}COR^{3d}, —NR^{3c}CO₂R^{3d} or —NR^{3c}SO₂R^{3d}, wherein each of said methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl, 3- to 8-membered heterocyclyl, phenyl, 5- to 12-membered heteroaryl is optionally substituted with —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —CN, —OR^{3e}, —SO₂R^{3e}, —SO₂NR^{3e}R^{3f}, —C(O)R^{3e}, —CO₂R^{3f}, —C(O)NR^{3e}R^{3f}, —NR^{3e}R^{3f}, —NR^{3e}COR^{3f}, —NR^{3e}CO₂R^{3f} or —NR^{3e}SO₂R^{3f};
R^{3c}, R^{3d}, R^{3e} and R^{3f} are each independently selected from hydrogen, methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl, wherein each of said methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —CN, —OH, methoxy, ethoxy, propoxy, butoxy, pentoxy, hexoxy, heptoxy, or octoxy;
preferably, R³ and R^{Q} are each independently selected from hydrogen, methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
more preferably, R³ and R^{Q} are each independently selected from hydrogen, methyl, ethyl, propyl (n-propyl or isopropyl), butyl (n-butyl, sec-butyl, iso-butyl or tert-butyl); and
even more preferably, R³ and R^{Q} are each independently hydrogen.

4. The compound of any one of the preceding claims, wherein R⁴ and R⁵ are each independently selected from hydrogen, —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl;
preferably, R⁴ and R⁵ are each independently selected from hydrogen, —F, —Cl, —Br, —I, methyl, ethyl, propyl (n-propyl or isopropyl), butyl (n-butyl, sec-butyl, iso-butyl or tert-butyl), methoxy, ethoxy, propoxy, cyclopropyl, cyclobutyl, cyclopentyl;
even more preferably, R⁴ and R⁵ are each independently selected from hydrogen.

5. The compound of any one of the preceding claims, wherein the 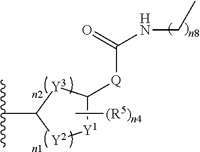
moiety is selected from 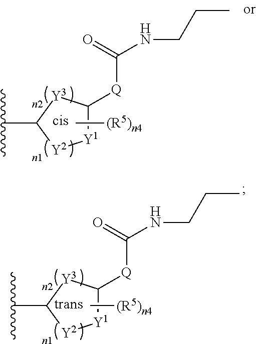
preferably, the 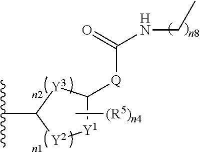
moiety is selected from 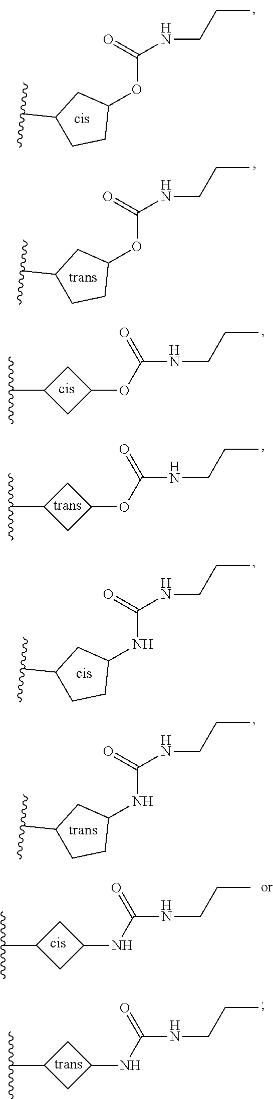
more preferably, the 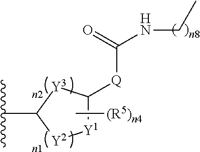
moiety is selected from 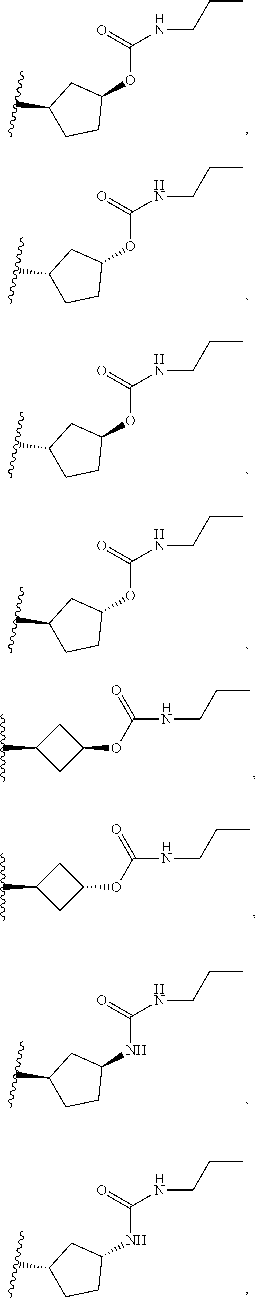 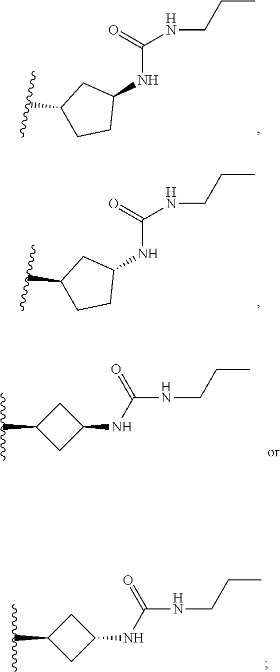 [SMILES: *[C@H]1CC[C@H](NC(=O)NCCC)C1 ; *[C@@H]1CC[C@@H](NC(=O)NCCC)C1 ; *[C@H]1C[C@@H](NC(=O)NCCC)C1 ; *[C@H]1C[C@H](NC(=O)NCCC)C1]
even more preferably, the 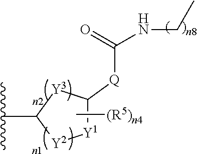
moiety is selected from

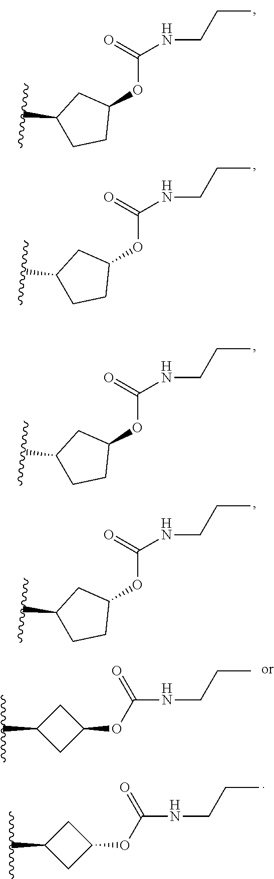

6. The compound of any one of the preceding claims, wherein at most two of Z¹, Z² and Z are N; preferably, at most one of Z¹, Z² and Z³ is N;
more preferably, Z¹, Z² and Z³ are independently —CR^{Z}.

7. The compound of any one of the preceding claims, wherein R^{Z}, at each occurrence, is independently selected from hydrogen, —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —NR^{Za}R^{Zb}, —OR^{Za}, —SR^{Za}, —SO₂R^{Za}, —SO₂NR^{Za}R^{Zb}, —C(O)R^{Za}, —CO₂R^{Za}, —C(O)NR^{Za}R^{Zb}, —NR^{Za}COR^{Zb}, —NR^{Za}CO₂R^{Zb} or —NR^{Za}SO₂R^{Zb} or —CN, wherein each of said methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one R^{Zc};
R^{Za} and R^{Zb} are each independently selected from hydrogen, methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl, wherein each of said methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one substituent R^{Zd};
R^{Zc} and R^{Zd} are each independently selected from —F, —Cl, —Br, —I, hydroxy, methyl, ethyl, propyl (n-propyl or isopropyl), butyl (n-butyl, sec-butyl, iso-butyl or tert-butyl), pentyl, hexyl, heptyl, octyl, methoxy, ethoxy, propoxy, butoxy, pentoxy, hexoxy, heptoxy, octoxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —NR^{Ze}R^{Zf}, —OR^{Ze}, —SR^{Ze} SO₂R^{Zf}, —SO₂NR^{Ze}R^{Zf}, —C(O)R^{Ze}, —CO₂R^{Ze}, —C(O)NR^{Ze}R^{Zf}, —NR^{Ze}CO₂R^{Zf}, —NR^{Ze}COR^{Zf} or —NR^{Ze}SO₂R^{Zf} or —CN;
R^{Ze} and R^{Zf} are each independently selected from methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl;
preferably, R^{Z}, at each occurrence, is independently selected from hydrogen, —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —CH₂F, —CHF₂, —CF₃, —CH₂CH₂F, —CH₂CHF₂, —CH₂CF₃, —NR^{Za}R^{Zb}, —OR^{Za}, —SR^{Za}, —SO₂R^{Za}, —SO₂NR^{Za}R^{Zb}, —C(O)R^{Za}, —CO₂R^{Za}, —C(O)NR^{Za}R^{Zb}, —NR^{Za}COR^{Zb}, —NR^{Za}CO₂R^{Zb} or —NR^{Za}SO₂R^{Zb} or —CN;
R^{Za} and R^{Zb} are each independently selected from hydrogen, methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
more preferably, R^{Z}, at each occurrence, is independently selected from hydrogen, —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —CH₂F, —CHF₂, —CF₃, —CH₂CH₂F, —CH₂CHF₂, —CH₂CF₃, —NH₂, —OH, methoxy, ethoxy, propoxy, butoxy, pentoxy, hexoxy, heptoxy, octoxy or —CN; and
even more preferably, R^{Z}, at each occurrence, is independently selected from hydrogen, —F, and methyl.

8. The compound of any one of the preceding claims, wherein the 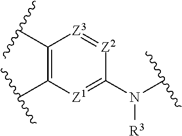
moiety is selected from 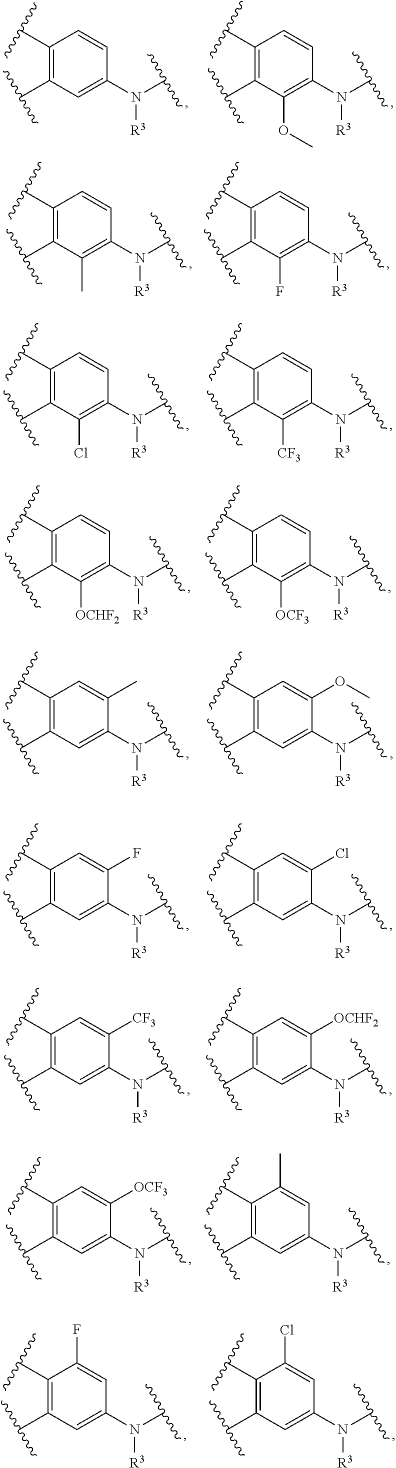 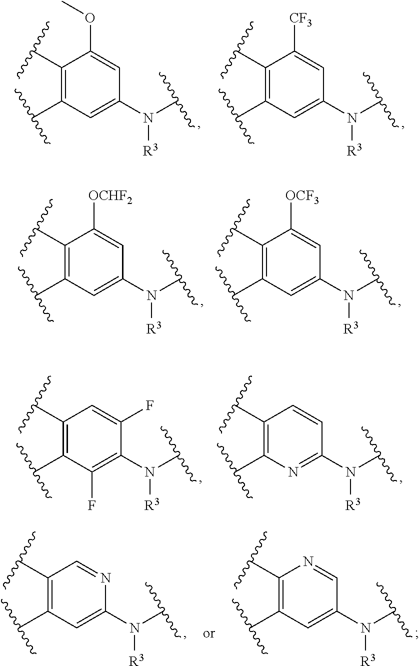
preferably, the 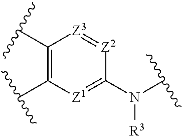
moiety is selected from

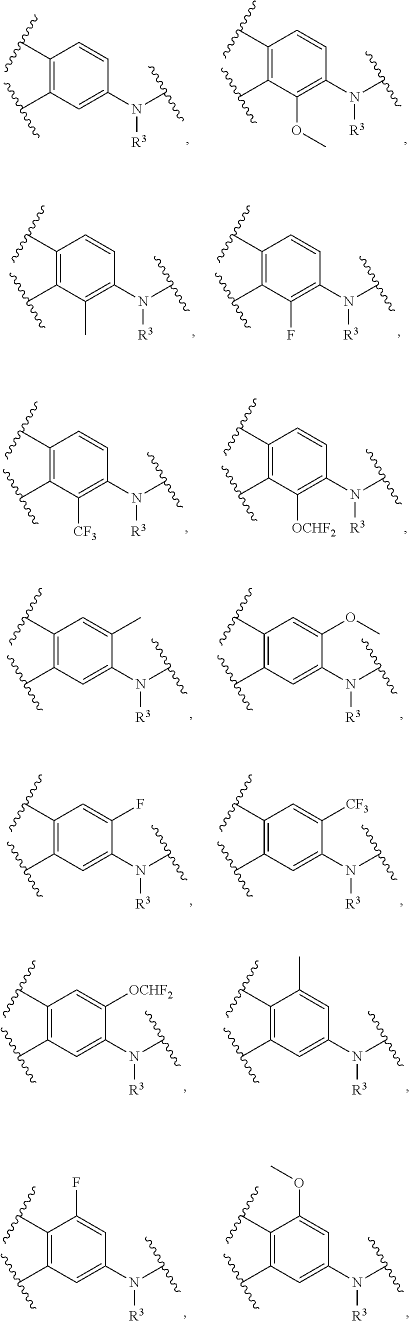

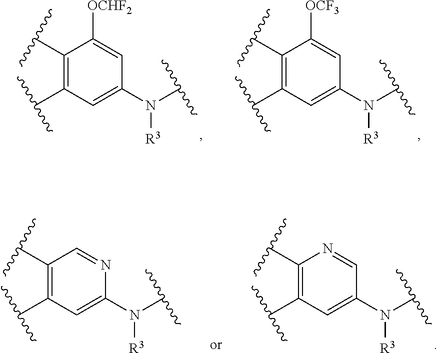

9. The compound of any one of the preceding claims, wherein R^{X} is each independently selected from hydrogen, —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —NR^{Xa}R^{Xb}, —OR^{Xa}, —SR^{Xa}, —SO₂R^{Xa}, —SO₂NR^{Xa}R^{Xb}, —C(O)R^{Xa}, —CO₂R^{Xa}, —C(O)NR^{Xa}R^{Xb}, —NR^{Xa}COR^{Xb}, —NR^{Xa}CO₂R^{Xb} or —NR^{Xa}SO₂R^{Xb} or —CN, wherein each of said methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one R^{Xc};
R^{Xa} and R^{Xb} are each independently selected from hydrogen, methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl, wherein each of said methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one substituent R^{Xd};
R^{Xc} and R^{Xd} are each independently selected from —F, —Cl, —Br, —I, hydroxy, methyl, ethyl, propyl (n-propyl or isopropyl), butyl (n-butyl, sec-butyl, iso-butyl or tert-butyl), pentyl, hexyl, heptyl, octyl, methoxy, ethoxy, propoxy, butoxy, pentoxy, hexoxy, heptoxy, octoxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), —NR^{Xe}R^{Xf}, —OR^{Xe}, —SR^{Xe}, —SO₂R^{Xe}, —SO₂NR^{Xe}R^{Xf}, —C(O)R^{Xe}, —CO₂R^{Xe}, —C(O)NR^{Xe}R^{Xf}, —NR^{Xe}COR^{Xf}, —NR^{Xe}CO₂R^{Xf} or —NR^{Xe}SO₂R^{Xf} or —CN;
R^{Xe} and R^{Xf} are each independently selected from methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl;
preferably, R^{X} is independently selected from hydrogen, —F, —Cl, —Br, —I, methyl, ethyl, propyl (n-propyl or isopropyl), butyl (n-butyl, sec-butyl, iso-butyl or tert-butyl), pentyl, hexyl, heptyl, octyl, cyclopropyl, cyclobutyl, cyclopentyl, cyclohexyl, cycloheptyl, cyclooctyl, 3- to 8-membered heterocyclyl, phenyl, 5- to 12-membered heteroaryl, —CH₂F, —CHF₂, —CF₃, —CH₂CH₂F, —CH₂CHF₂, —CH₂CF₃, oxo (═O), —NR^{Xa}R^{Xb}, —OR^{Xa}, —SR^{Xa}, —SO₂R^{Xa}, —SO₂NR^{Xa}R^{Xb}, —C(O)R^{Xa}, —CO₂R^{Xa}, —C(O)NR^{Xa}R^{Xb}, —NR^{Xa}COR^{Xb}, —NR^{Xa}CO₂R^{Xb} or —NR^{Xa}SO₂R^{Xb} or —CN;
R^{Xa} and R^{Xb} are each independently selected from hydrogen, methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
more preferably, R^{X} is independently selected from hydrogen, —F, —Cl, methyl, ethyl, propyl (n-propyl or isopropyl), butyl (n-butyl, sec-butyl, iso-butyl or tert-butyl), cyclopropyl, cyclobutyl, cyclopentyl, cyclohexyl, —CH₂F, —CHF₂, —CF₃, —CH₂CH₂F, —CH₂CHF₂, —CH₂CF₃, oxo (═O), or —OH; and
even more preferably, R^{X} is hydrogen.

10. The compound of any one of the preceding claims, wherein two RN together with the atom(s) to which they are attached, form a 3-, 4-, 5-, 6-, 7- or 8-membered ring, said ring comprising 0, 1, 2 or 3 heteroatoms independently selected from nitrogen, oxygen or optionally oxidized sulfur as ring member(s); said ring is optionally substituted with at least one substituent R^{Xc};
R^{Xc} is each independently selected from —F, —Cl, —Br, —I, hydroxy, methyl, ethyl, propyl (n-propyl or isopropyl), butyl (n-butyl, sec-butyl, iso-butyl or tert-butyl), pentyl, hexyl, heptyl, octyl, methoxy, ethoxy, propoxy, butoxy, pentoxy, hexoxy, heptoxy, octoxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (=)), —NR^{Xe}R^{Xf}, —OR^{Xe}, —SR^{Xe}, —SO₂R^{Xe}, —SO₂NR^{Xe}R^{Xf}, —C(O)R^{Xe}, —CO₂R^{Xe}, —C(O)NR^{Xe}R^{Xf}, —NR^{Xe}COR^{Xf}, —NR^{Xe}CO₂R^{Xf} or —NR^{Xe}SO₂R^{Xf} or —CN;
R^{Xe} and R^{Xf} are each independently selected from methyl, ethyl, propyl (n-propyl or isopropyl), butyl (n-butyl, sec-butyl, iso-butyl or tert-butyl), pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, 5- to 12-membered heteroaryl;
preferably, two R^{X} together with the atom(s) to which they are attached, form a 3-, 4-, 5- or 6-membered ring; said ring is optionally substituted with at least one substituent R^{Xc};
R^{Xc} is each independently selected from —F, —Cl, —Br, —I, hydroxy, methyl, ethyl, propyl (n-propyl or isopropyl), butyl (n-butyl, sec-butyl, iso-butyl or tert-butyl), methoxy, ethoxy, propoxy, butoxy, cyclopropyl, cyclobutyl, cyclopentyl, cyclohexyl, oxo (═O) or —CN;
more preferably, two R^{X} together with the atom(s) to which they are attached, form a 3-, 4-, 5- or 6-membered ring.

11. The compound of any one of the preceding claims, wherein the 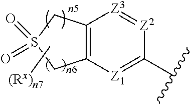
moiety is selected from

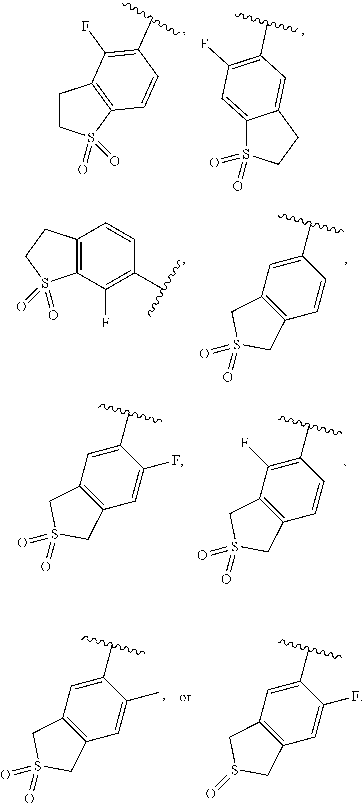

12. A compound, or a N-oxide thereof, or a pharmaceutically acceptable salt thereof, or a stereoisomer thereof, or a tautomer, or a deuterated analog thereof, or a prodrug thereof, wherein the compound is selected from

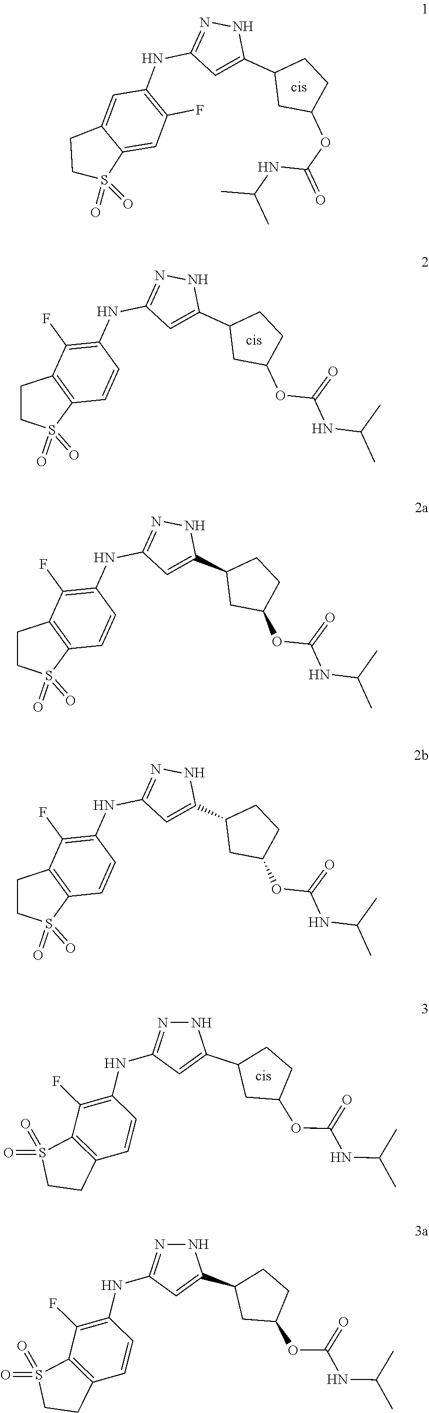 [SMILES: CC(C)NC(=O)OC1CCC(c2cc(Nc3cc4c(cc3F)S(=O)(=O)CC4)n[nH]2)C1 ; CC(C)NC(=O)OC1CCC(c2cc(Nc3ccc4c(c3F)CCS4(=O)=O)n[nH]2)C1 ; CC(C)NC(=O)O[C@@H]1CC[C@H](c2cc(Nc3ccc4c(c3F)CCS4(=O)=O)n[nH]2)C1 ; CC(C)NC(=O)O[C@H]1CC[C@@H](c2cc(Nc3ccc4c(c3F)CCS4(=O)=O)n[nH]2)C1 ; CC(C)NC(=O)OC1CCC(c2cc(Nc3ccc4c(c3F)S(=O)(=O)CC4)n[nH]2)C1 ; CC(C)NC(=O)O[C@@H]1CC[C@H](c2cc(Nc3ccc4c(c3F)S(=O)(=O)CC4)n[nH]2)C1]

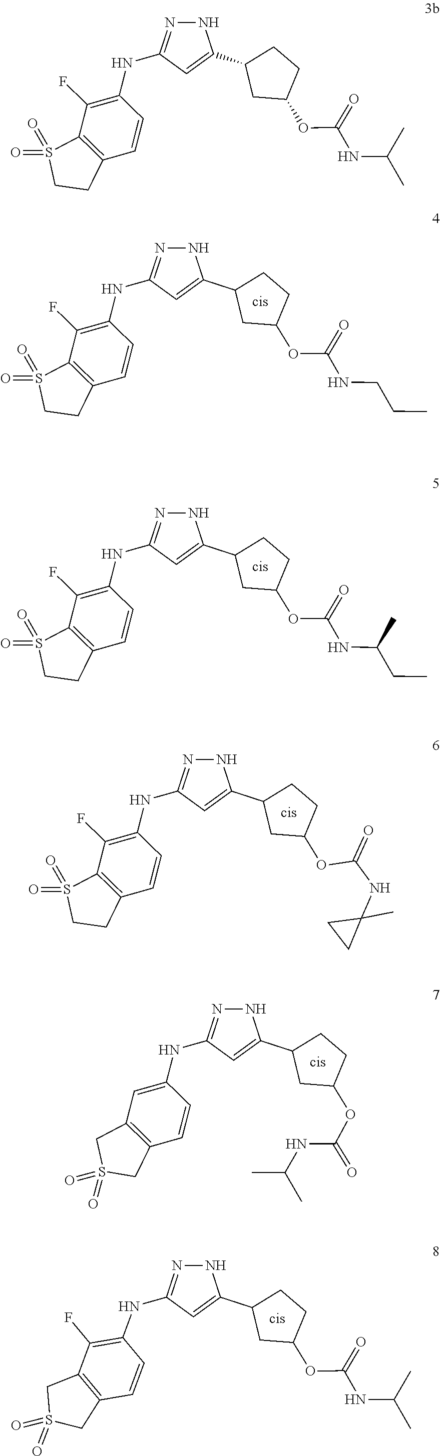 [SMILES: CC(C)NC(=O)O[C@H]1CC[C@@H](c2cc(Nc3ccc4c(c3F)S(=O)(=O)CC4)n[nH]2)C1 ; CCCNC(=O)OC1CCC(c2cc(Nc3ccc4c(c3F)S(=O)(=O)CC4)n[nH]2)C1 ; CC[C@H](C)NC(=O)OC1CCC(c2cc(Nc3ccc4c(c3F)S(=O)(=O)CC4)n[nH]2)C1 ; CC1(NC(=O)OC2CCC(c3cc(Nc4ccc5c(c4F)S(=O)(=O)CC5)n[nH]3)C2)CC1 ; CC(C)NC(=O)OC1CCC(c2cc(Nc3ccc4c(c3)CS(=O)(=O)C4)n[nH]2)C1 ; CC(C)NC(=O)OC1CCC(c2cc(Nc3ccc4c(c3F)CS(=O)(=O)C4)n[nH]2)C1]

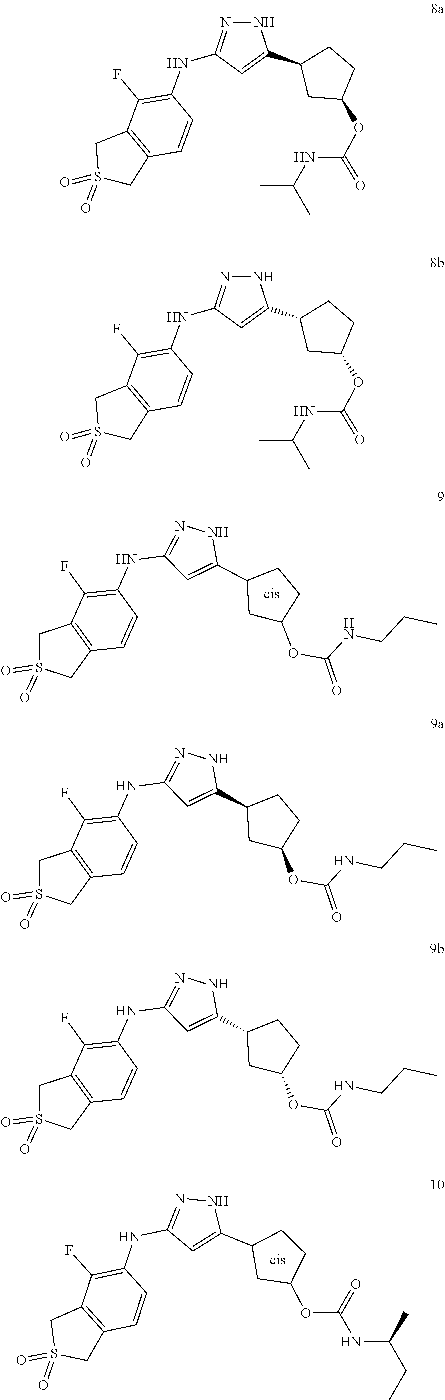 [SMILES: CC(C)NC(=O)O[C@@H]1CC[C@H](c2cc(Nc3ccc4c(c3F)CS(=O)(=O)C4)n[nH]2)C1 ; CC(C)NC(=O)O[C@H]1CC[C@@H](c2cc(Nc3ccc4c(c3F)CS(=O)(=O)C4)n[nH]2)C1 ; CCCNC(=O)OC1CCC(c2cc(Nc3ccc4c(c3F)CS(=O)(=O)C4)n[nH]2)C1 ; CCCNC(=O)O[C@@H]1CC[C@H](c2cc(Nc3ccc4c(c3F)CS(=O)(=O)C4)n[nH]2)C1 ; CCCNC(=O)O[C@H]1CC[C@@H](c2cc(Nc3ccc4c(c3F)CS(=O)(=O)C4)n[nH]2)C1 ; CC[C@H](C)NC(=O)OC1CCC(c2cc(Nc3ccc4c(c3F)CS(=O)(=O)C4)n[nH]2)C1]

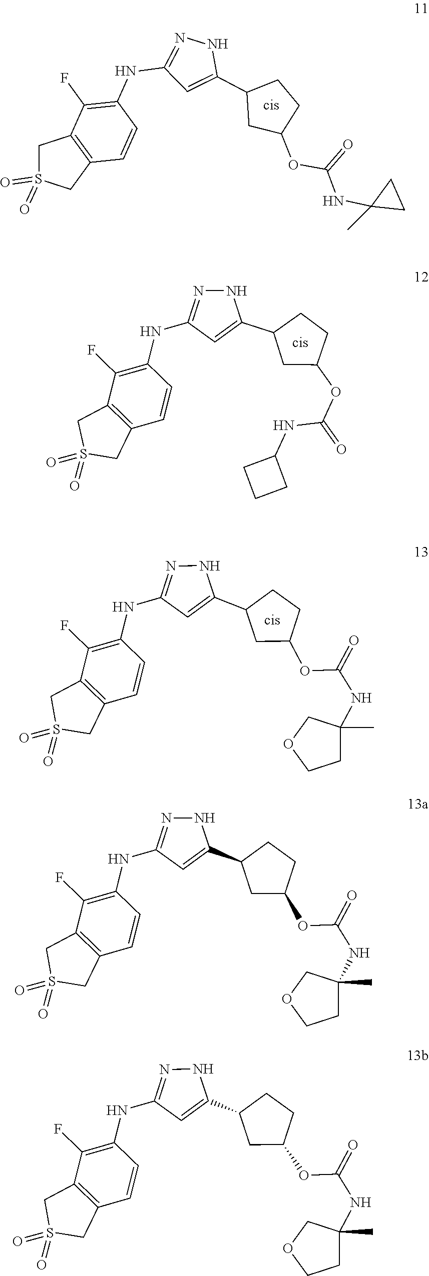 [SMILES: CC1(NC(=O)OC2CCC(c3cc(Nc4ccc5c(c4F)CS(=O)(=O)C5)n[nH]3)C2)CC1 ; O=C(NC1CCC1)OC1CCC(c2cc(Nc3ccc4c(c3F)CS(=O)(=O)C4)n[nH]2)C1 ; CC1(NC(=O)OC2CCC(c3cc(Nc4ccc5c(c4F)CS(=O)(=O)C5)n[nH]3)C2)CCOC1 ; C[C@@]1(NC(=O)O[C@@H]2CC[C@H](c3cc(Nc4ccc5c(c4F)CS(=O)(=O)C5)n[nH]3)C2)CCOC1 ; C[C@@]1(NC(=O)O[C@H]2CC[C@@H](c3cc(Nc4ccc5c(c4F)CS(=O)(=O)C5)n[nH]3)C2)CCOC1]

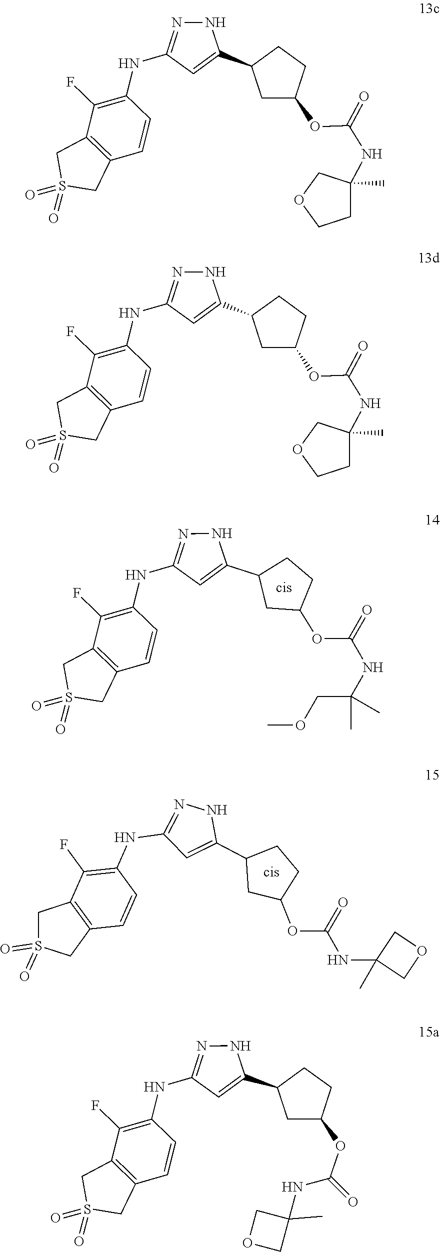 [SMILES: C[C@]1(NC(=O)O[C@@H]2CC[C@H](c3cc(Nc4ccc5c(c4F)CS(=O)(=O)C5)n[nH]3)C2)CCOC1 ; C[C@]1(NC(=O)O[C@H]2CC[C@@H](c3cc(Nc4ccc5c(c4F)CS(=O)(=O)C5)n[nH]3)C2)CCOC1 ; COCC(C)(C)NC(=O)OC1CCC(c2cc(Nc3ccc4c(c3F)CS(=O)(=O)C4)n[nH]2)C1 ; CC1(NC(=O)OC2CCC(c3cc(Nc4ccc5c(c4F)CS(=O)(=O)C5)n[nH]3)C2)COC1 ; CC1(NC(=O)O[C@@H]2CC[C@H](c3cc(Nc4ccc5c(c4F)CS(=O)(=O)C5)n[nH]3)C2)COC1]

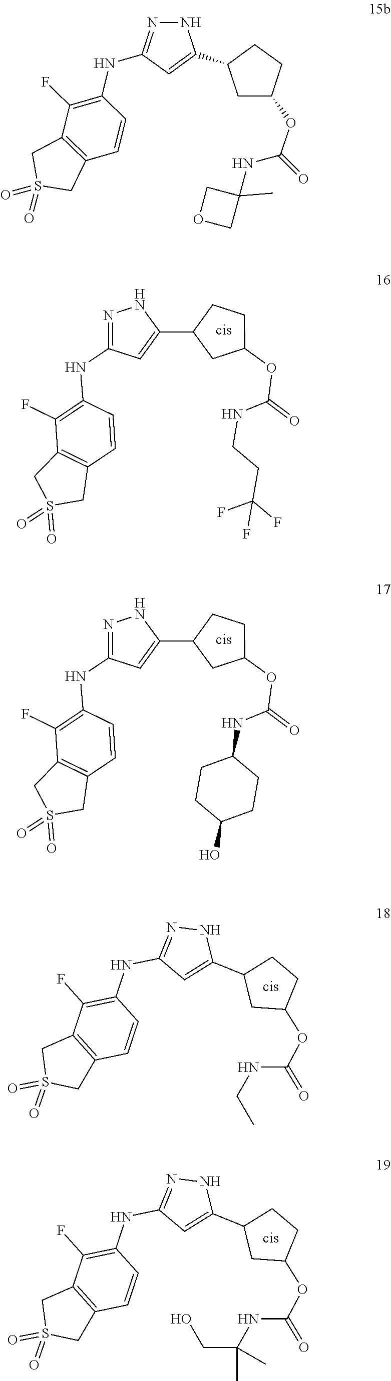 [SMILES: CC1(NC(=O)O[C@H]2CC[C@@H](c3cc(Nc4ccc5c(c4F)CS(=O)(=O)C5)n[nH]3)C2)COC1 ; O=C(NCCC(F)(F)F)OC1CCC(c2cc(Nc3ccc4c(c3F)CS(=O)(=O)C4)n[nH]2)C1 ; O=C(N[C@H]1CC[C@@H](O)CC1)OC1CCC(c2cc(Nc3ccc4c(c3F)CS(=O)(=O)C4)n[nH]2)C1 ; CCNC(=O)OC1CCC(c2cc(Nc3ccc4c(c3F)CS(=O)(=O)C4)n[nH]2)C1 ; CC(C)(CO)NC(=O)OC1CCC(c2cc(Nc3ccc4c(c3F)CS(=O)(=O)C4)n[nH]2)C1]

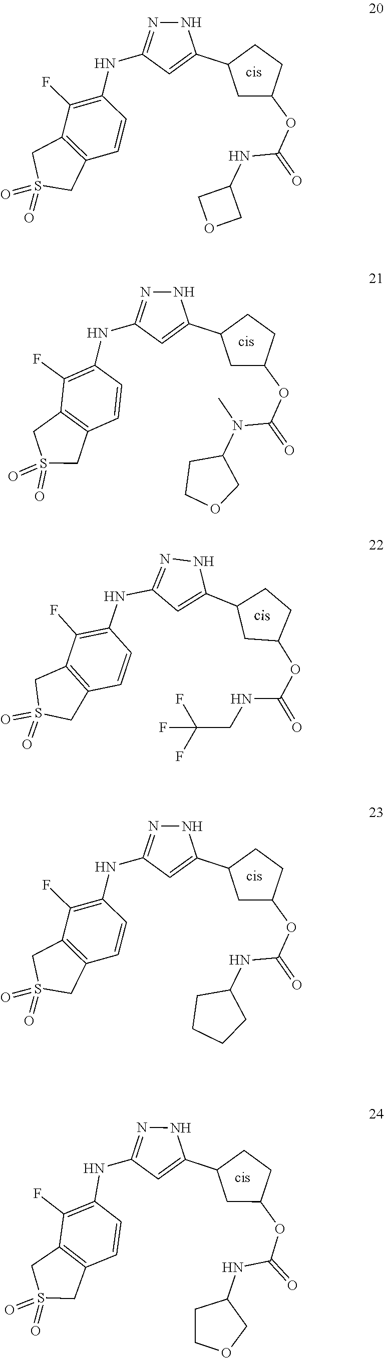 [SMILES: O=C(NC1COC1)OC1CCC(c2cc(Nc3ccc4c(c3F)CS(=O)(=O)C4)n[nH]2)C1 ; CN(C(=O)OC1CCC(c2cc(Nc3ccc4c(c3F)CS(=O)(=O)C4)n[nH]2)C1)C1CCOC1 ; O=C(NCC(F)(F)F)OC1CCC(c2cc(Nc3ccc4c(c3F)CS(=O)(=O)C4)n[nH]2)C1 ; O=C(NC1CCCC1)OC1CCC(c2cc(Nc3ccc4c(c3F)CS(=O)(=O)C4)n[nH]2)C1 ; O=C(NC1CCOC1)OC1CCC(c2cc(Nc3ccc4c(c3F)CS(=O)(=O)C4)n[nH]2)C1]

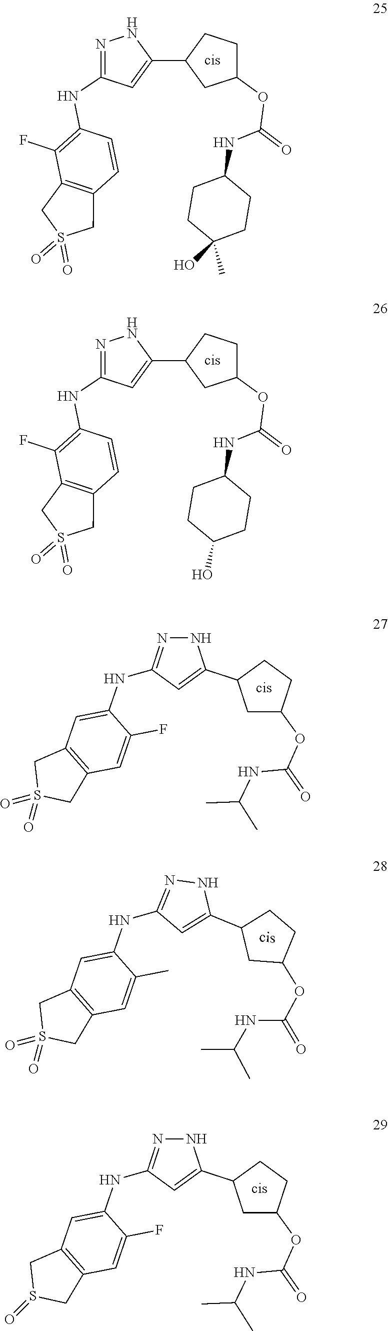 [SMILES: C[C@]1(O)CC[C@@H](NC(=O)OC2CCC(c3cc(Nc4ccc5c(c4F)CS(=O)(=O)C5)n[nH]3)C2)CC1 ; O=C(N[C@H]1CC[C@H](O)CC1)OC1CCC(c2cc(Nc3ccc4c(c3F)CS(=O)(=O)C4)n[nH]2)C1 ; CC(C)NC(=O)OC1CCC(c2cc(Nc3cc4c(cc3F)CS(=O)(=O)C4)n[nH]2)C1 ; Cc1cc2c(cc1Nc1cc(C3CCC(OC(=O)NC(C)C)C3)[nH]n1)CS(=O)(=O)C2 ; CC(C)NC(=O)OC1CCC(c2cc(Nc3cc4c(cc3F)CS(=O)C4)n[nH]2)C1]

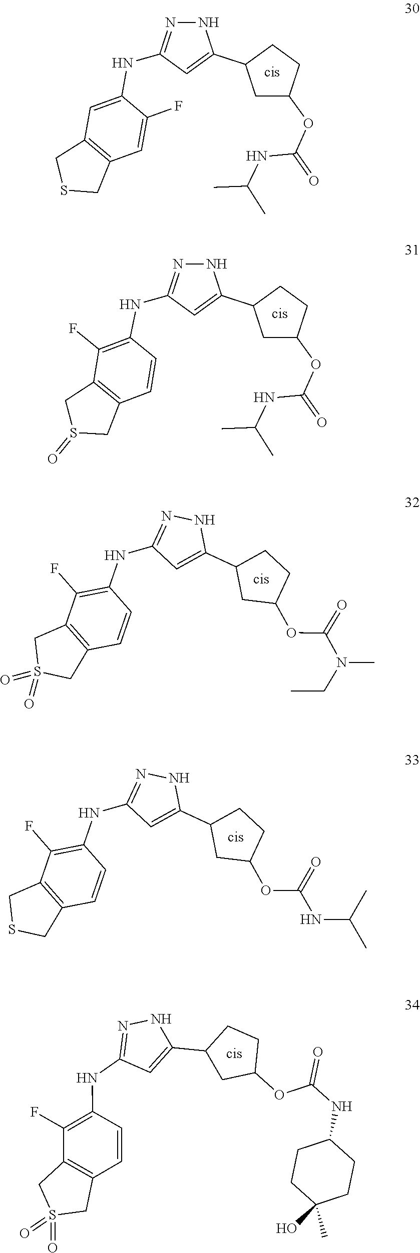 [SMILES: CC(C)NC(=O)OC1CCC(c2cc(Nc3cc4c(cc3F)CSC4)n[nH]2)C1 ; CC(C)NC(=O)OC1CCC(c2cc(Nc3ccc4c(c3F)CS(=O)C4)n[nH]2)C1 ; CCN(C)C(=O)OC1CCC(c2cc(Nc3ccc4c(c3F)CS(=O)(=O)C4)n[nH]2)C1 ; CC(C)NC(=O)OC1CCC(c2cc(Nc3ccc4c(c3F)CSC4)n[nH]2)C1 ; C[C@]1(O)CC[C@H](NC(=O)OC2CCC(c3cc(Nc4ccc5c(c4F)CS(=O)(=O)C5)n[nH]3)C2)CC1]

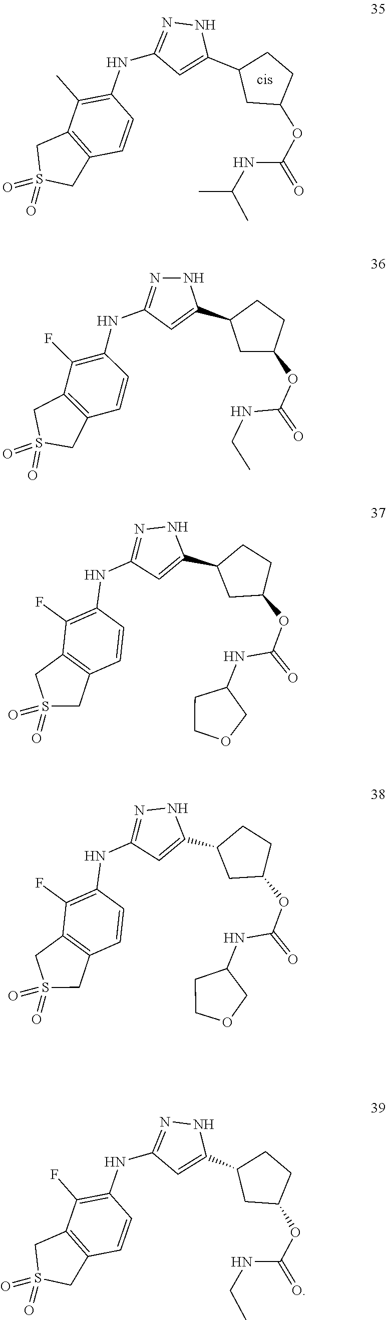 [SMILES: Cc1c(Nc2cc(C3CCC(OC(=O)NC(C)C)C3)[nH]n2)ccc2c1CS(=O)(=O)C2 ; CCNC(=O)O[C@@H]1CC[C@H](c2cc(Nc3ccc4c(c3F)CS(=O)(=O)C4)n[nH]2)C1 ; O=C(NC1CCOC1)O[C@@H]1CC[C@H](c2cc(Nc3ccc4c(c3F)CS(=O)(=O)C4)n[nH]2)C1 ; O=C(NC1CCOC1)O[C@H]1CC[C@@H](c2cc(Nc3ccc4c(c3F)CS(=O)(=O)C4)n[nH]2)C1 ; CCNC(=O)O[C@H]1CC[C@@H](c2cc(Nc3ccc4c(c3F)CS(=O)(=O)C4)n[nH]2)C1]

13. A pharmaceutical composition comprising a compound of any one of claims 1-12, or a pharmaceutically acceptable salt thereof, or a stereoisomer, a tautomer or a prodrug thereof, and at least one pharmaceutically acceptable carrier or excipient.

14. A method of treating cancer, comprising administering to a subject in need thereof a compound of any one of claims 1-12, or a pharmaceutically acceptable salt, or a stereoisomer, a tautomer or a prodrug thereof.
